(19) United States
(12) Patent Application Publication
(10) Pub. No.: US 2022/0226340 A1
(43) Pub. Date: Jul. 21, 2022

(54) CASEIN KINASE 1 INHIBITORS FOR USE IN THE TREATMENT OF DISEASES RELATED TO DUX4 EXPRESSION SUCH AS MUSCULAR DYSTROPHY AND CANCER

(71) Applicant: Facio Intellectual Property B.V., Leiden (NL)

(72) Inventors: Joris De Maeyer, Mechelen (BE); Marcus Geese, Göttingen (DE); Sebastian Monecke, Göttingen (DE); Rolf Hirsch, Göttingen (DE); Pui Leng Loke, Abingdon (GB)

(73) Assignee: Facio Intellectual Property B.V., Leiden (NL)

(21) Appl. No.: 17/617,321

(22) PCT Filed: Jun. 12, 2020
(86) PCT No.: PCT/EP2020/066280

§ 371 Date: Dec. 8, 2021

(30) Foreign Application Priority Data
Jun. 13, 2019 (EP) 19180120.8

(51) Int. Cl. A61K 31/5377; A61K 31/5025; A61K 31/496; A61K 31/506; A61K 31/437

(52) U.S. Cl. CPC A61K 31/5377; A61K 31/5025; A61K 31/437; A61K 31/506; A61K 31/496

(57) ABSTRACT

The present invention relates to compounds for the treatment of diseases related to DUX4 expression, such as muscular dystrophies. It also relates to use of such compounds, or to methods of use of such compounds.

### FIELD OF THE INVENTION

The present invention relates to combinations of compounds for the treatment of diseases related to DUX4 expression, such as muscular dystrophies. It also relates to use of such combinations, or to methods of use of such combinations.

### BACKGROUND ART

Facioscapulohumeral muscular dystrophy (FSHD) is one of the most prevalent hereditary muscular dystrophies. Symptoms begin before the age of 20, with weakness and atrophy of the muscles around the eyes and mouth, shoulders, upper arms and lower legs. Later, weakness can spread to abdominal muscles and sometimes hip muscles with approximately 20% of patients eventually becoming wheelchair-bound. Patients currently rely on treatment of symptoms like pain and fatigue, involving the use of pain medication, cognitive therapy, and physical exercise, sometimes supplemented with medical devices used to maintain the patient's mobility. Furthermore, increased scapular function may be obtained by surgical treatment of the scapula. At best, these interventions remain symptomatic in nature and do not affect disease progression, illustrating the need for a therapy that is able to modify disease progression.

Significant progress has been made in recent years in the understanding of the molecular basis of FSHD resulting in the identification and characterization of the fundamental genetic lesions causing FSHD. There is the wide support for the pathogenesis model in which epigenetic de-repression of the Double Homeobox 4 (DUX4) transcription factor in muscle cells triggers pathology by initiating a transcription deregulation cascade that causes muscle atrophy, inflammation, and oxidative stress, which can be key features of the disease (Lemmers et al., 2010, DOI: 10.1126/science.1189044; Sharma et al., 2016, DOI:10.4172/2157-7412.1000303, Snider et al., 2010, DOI: 10.1371/journal.pgen.1001181; Tawil et al., 2014, DOI: 10.1186/2044-5040-4-12).

FSHD is sometimes divided in two subtypes, namely FSHD1 and FSHD2. FSHD1 is associated with large deletions within a DNA tandem array (D4Z4) that is located in the subtelomeric region of chromosome 4q35. Each of the D4Z4 repeats contains a copy of the DUX4 gene. In healthy individuals, DUX4 is expressed in the germline, but is epigenetically silenced in somatic tissues. Healthy, genetically unaffected individuals are defined as having between 10 and 100 D4Z4 repeat units on both 4q chromosome arms, whereas individuals with FSHD1 have between 1 and 10 D4Z4 repeat units on one 4q chromosome arm. The deletions of D4Z4 repeats that characterize FSHD remove a substantial portion of regulatory chromatin from this region, including several hundreds of histones and a significant amount of CpG-rich DNA. These elements are essential in the establishment of DNA methylation and heterochromatin and their loss significantly alters the epigenetic status of the D4Z4 array. The contraction of D4Z4 is by itself not pathogenic. Only when the contraction of D4Z4 occurs on a disease-permissive 4qA allele, containing a polymorphism that could affect the polyadenylation of the distal DUX4 transcript, the altered epigenetic context is associated with alternative splicing and increased expression of DUX4 in skeletal muscles of FSHD1 patients. In the much rarer form FSHD2, the cause is a mutated form of an epigenetic factor such as SMCHD1 or DNMT3B. In this form as well, the D4Z4 region is hypomethylated and muscle cells are characterized by a de-repressed DUX4 protein. It has been suggested that FSHD1 and FSHD2 are on a continuum, rather than being separate (Van den Boogaard et al., 2016, DOI: 10.1016/j.ajhg.2016.03.013). Both forms of FSHD converge on undue DUX4 expression. Burst-like DUX4 expression in only a small fraction of myofibers causes myocyte death ultimately leading to muscle weakness and wasting (Lemmers et al., 2010).

In the simplest terms, DUX4-overexpression is a primary pathogenic insult underlying FSHD, and its repression is a promising therapeutic approach for FSHD. In support of this, short repeat sizes are generally associated with a severe FSHD phenotype. Moderate repeat contractions have a milder and more variable clinical severity. Also in FSHD2, the D4Z4 region is hypomethylated and muscle cells are characterized by a de-repressed DUX4 protein. Patients with less than 10 D4Z4 repeat units that also have a mutation in SMCHD1 have a very severe clinical phenotype, illustrating that a combination of repeat size and activity of epigenetic modifiers, both contributing to derepression of DUX4, determines the eventual disease severity in FSHD. DUX4 acts as a transcription factor whose expression initiates a transcription cascade resulting in progressive muscle cell dysfunction and death, and ultimately to overt pathology (Kowaljow et al., 2007, DOI: 10.1016/j.nmd.2007.04.002 ; Vanderplanck et al., 2011, doi: 10.1371/journal.pone.0026820 ; Geng et al., 2012, DOI: 10.1016/j.devce1.2011.11.013 ; Yao et al., 2014, DOI: 10.1093/hmg/ddu251 ; Wallace et al., 2011, DOI: 10.1002/ana.22275).

Because of its causative role in FSHD, suppressing DUX4 is a primary therapeutic approach for halting disease progression. This approach could also be useful for treating other diseases, such as cancers including acute lymphoblastic leukemia (Yasuda et al., 2016, doi: 10.1038/ng.3535) and sarcomas (Oyama et al., 2017 DOI: 10.1038/s41598-017-04967-0; Bergerat et al., 2017, DOI: 10.1016/j.prp.2016.11.015), etc. However, the mechanisms behind DUX4 expression are poorly understood and corresponding drug targets are poorly defined. As a result, there is no treatment for FSHD at present, and there is a need for compounds and compositions that can be used to suppress DUX4 expression. Campbell et al. (2017, DOI 10.1186/s13395-017-0134-x) screened a selection of chemical compounds with known epigenetic activities as well as the Pharmakon 1600 library composed of compounds that have reached clinical testing to identify molecules that decrease DUX4 expression as monitored by the expression levels of DUX4 target gene mRNAs in immortalized FSHD skeletal muscle cell cultures. They identified several classes of molecules that include inhibitors of the bromodomain and extra-terminal (BET) family of proteins and agonists of the beta-2 adrenergic receptor. Their studies suggest that compounds from these two classes suppress the expression of DUX4 by blocking the activity of bromodomain-containing protein 4 (BRD4) or by increasing cyclic adenosine monophosphate (cAMP) levels, respectively. WO2019/071144 and WO2019/071147 suggest the use of p38 inhibitors such as losmapimod for the reduction of DUX4 expression in the context of FSHD treatment.

Measuring the severity and progression of FSHD is particularly challenging because, unlike most muscular dystrophies that progress symmetrically at a constant rate, FSHD is characterized by stepwise, asymmetric progression of muscle wasting, and weakness. Therefore, outcome measures and biomarkers to stratify patients are essential for measuring disease burden and testing effects of interventions. As in the other muscular dystrophies, muscle tissue is replaced by fat during the course of the disease. The degree of fatty infiltration, as measured by MRI, correlates with a commonly used measure of functional status, the FSHD clinical severity score (Mul et al., 2017, DOI: 10.1212/WNL.0000000000004647). Furthermore, it has been proposed that muscle inflammation contributes to the pathophysiology of FSHD and that it precedes the muscle destruction and fatty replacement, thereby representing an early marker for disease activity. Muscle inflammation can be investigated using MRI sequences with short TI inversion recovery (STIR). STIR hyperintensities (STIR+) visualize edema. An immune-mediated mechanism for FSHD is consistent with the focal inflammation and CD8+ T cell infiltrates that characterize affected FSHD muscle biopsies (Wang et al., Hum Mol Genet. 2019 February 1;28(3):476-486. doi: 10.1093/hmg/ddy364, Frisullo et al., 2011, DOI: 10.1007/s1087 5-010-9474-6). Prominent inflammatory cellular infiltrates, mimicking inflammatory myopathies are often observed in STIR+ muscle biopsies and it has indeed been suggested that this can represent an early marker of active disease (Geng et al. 2012, DOI: 10.1016/j.devce1.2011.11.013). Therefore, MRI with STIR sequences has been proposed as a prognostic outcome measure (Tasca et al., 2016, DOI: 10.1002/ana.24640; Janssen et al., 2014, DOI: 10.1371/journ al.pone.00854 16). Studies that have investigated muscle inflammation and fat replacement in FSHD have suggested that severe muscle inflammation predicts a faster fat replacement of muscle (Tasca et al., 2016, DOI: 10.1002/ana.24640; Ferguson et al., 2018, DOI: 10.1002/mus.26038; Wang et al., 2019, supra). It has been suggested that, once a muscle reaches an inter-mediate fat fraction, it accelerates towards complete fat replacement and that muscle inflammation might act as a trigger for this process (Janssen et al., 2014, DOI: 10.1371/journal.pone.00854 16).

A recent study investigated the pathophysiology of FSHD in 45 patients over 14 months by quantifying STIR hyperintensities, indicating inflammation, and fat content in thigh muscles and investigated its relation and progression over 14 months (Dahlqvist et al., 2019, 10.1007/s00415-019-09242-y). Forty-three patients were included in the muscle inflammation and fat content analyses and nine thigh muscles were evaluated in each patient. Thirty-three patients had at least one STIR+ muscle at baseline and 34 at follow-up. Of all 370 analyzed muscles, 83 were STIR+ at baseline and 103 at follow-up. All muscles had a significant increase in fat content over the 14 months. This progression of fat replacement was more than twice as fast in STIR+ muscles compared to STIR− muscles and increased with the severity of the hyperintensity. This illustrates that patients with active inflammation have muscle fibers that are undergoing active disease progression.

Interestingly, a recent study examined the correlation between MRI changes, corresponding pathologic changes, and DUX4-target gene expression in FSHD. The cross-sectional data showed that STIR+ MRI measures might have substantial predictive value for identifying muscles with DUX4 expression and active disease. Indeed, using an elevated STIR rating to select muscles with increased DUX4 target expression DUX4 expression was observed in ˜90% of the samples (Wang et al., supra). In addition, biopsies taken in STIR+ muscles showed gene expression changes that can be assigned to inflammation, extracellular matrix remodeling and muscle regeneration (Tasca et al., 2012, DOI: doi.org/10.1371/journal.pone.0038779). This suggests a causal association between the inflammatory infiltrate and DUX4 expression. Many of the genes highly upregulated by DUX4 normally function in the germline and/or early stem cells and are not present in healthy adult skeletal muscle. Activation of the gametogenic program is thought to be incompatible with postmitotic skeletal muscle, leading to apoptosis or cellular dysfunction.

While therapeutic approaches that cause reduction of DUX4 would be expected to impact downstream inflammatory, fatty infiltration and fibrotic processes in FSHD, patients with signs of active disease, which is characterized by both DUX4 expression and inflammation would benefit from treatments that affect both of these hallmarks. WO2019/071144 and WO2019/071147 disclose the use of p38 inhibitors such as losmapimod for the reduction of DUX4 expression in the context of FSHD treatment. p38 inhibitors have widely been in development for the treatment of inflammatory diseases such as chronic obstructive pulmonary disease (COPD) and reumathoid arthritis. In the innate immune response mitogen-activated protein kinase (MAPK) and nuclear factor-kB (NF-kB) are activated by pattern recognition receptors (PRRs) upon binding of the pathogen-associated molecular patterns (PAMPs). The fourteen MAPKs discovered in mammalian cells are crucial to generating immune responses. Altogether they play pivotal roles in the MAPK signal transduction pathway that cells use to adapt to inflammatory and stressful conditions. Pathogens or inflammatory stimuli initiate a phosphorylation cascade mediated by p38 kinase that leads to the transcription and translation of inflammatory response-associated genes that encode proteins such as TNF-a, IL-1b, IL-6, and IL-8 (Xing 2105, DOI: 10.4081/mk.2015.5508). Theoretically, p38 inhibitors could therefore affect both the expression of DUX4 as well as the local inflammation in FSHD muscle, which is a marker of disease with increased DUX4 expression and active regeneration (Wang et al., 2019, supra; Tasca et al., 2012, DOI: doi.org/10.1371/journal.pone.0038779).

However, p38α MAPK is known to play critical roles in skeletal muscle biology, specifically in abrogating proliferating myoblasts to differentiation and subsequently fusion to form multi-nucleated myotubes, which are undesirable effects in treating FSHD. In WO2019/071144 and WO2019/071147 data generated using immortalized myoblasts was used to suggest that p38a and p38b kinase inhibitors do not impact myogenin or the expression of other myogenic factors, and do not impact proliferation of myoblasts or differentiation of myoblasts exhibited by myogenic fusion in immortalized FSHD myotubes. However, the present inventors have found that p38 inhibitors do inhibit myogenic fusion of primary patient-derived FSHD myotubes. While expression of myogenic differentiation markers such as myogenin or MYH2 remained unchanged, inhibition of myogenic fusion was detected by high content image analysis of fusing primary myotube cultures where p38 inhibitors impaired myotube fusion.

There therefore is a clear need for compounds that both inhibit DUX4 and/or the inflammatory response in FSHD without affecting myotube formation. There is a need for compounds that reduce inhibition of myotube formation.

The present invention is based on the surprising discovery that inhibitors of casein kinase 1 (CK1, also known as CSNK1) can protect against negative effects on myotube formation. Combined inhibition of CK1 and p38 resulted in DUX4 inhibition without negatively affecting myotube formation. This beneficial phenotype could be observed by combining a CK1 inhibitor with a p38 inhibitor.

Serine/threonine kinases (EC 2.7.11.1) are a class of protein kinases that are promising drug targets for small molecule inhibitors. Due to their involvement in various signaling pathways in eukaryotic cells, inhibition of serine/threonine kinases is likely to have relevance to the treatment of diseases such as cancer, diabetes, and a variety of inflammatory disorders.

Casein kinase 1 (CK1, also known as CSNK1) belongs to the serine/threonine kinase family. Casein kinase 1 (CK1) is a ubiquitously expressed serine/threonine kinase family in mammals, known to phosphorylate a broad range of proteins. Accordingly, CK1 isoforms play essential regulatory roles in diverse cellular processes including proliferation, DNA repair, apoptosis, cell differentiation, circadian rhythm, Wnt signaling, nucleo-cytoplasmic shuttling of transcription factors and DNA transcription (Eide E J, Virshup D M (2001) doi:10.1081/CBI-100103963). The CK1 family consists of different isoforms (α, γ1, γ2, γ3, ε, and in humans, and their various alternative splice variants. CK1 isoforms possess a highly conserved kinase domain but differ significantly in length and composition of their N- and C-terminal sequences. The C-terminal domain has a plurality of autophosphorylation sites and is considered to be involved in regulation of autoenzyme activity. In mammals, the enzyme exists in seven isoforms: α, β, γ1, γ2, γ3, δ, and ε, all having a similar kinase domain. Through phosphorylation of different substrate proteins, these isoforms are able to activate, stabilize, inactivate, or destabilize the functions of these substrate proteins, thus regulating their functions. For example, a tumor suppressor factor p53 and an oncogene mdm2, which are both important proteins for controlling abnormal cell growth, are substrates of CK1. Phosphorylation of p53 by casein kinases such as casein kinase 1δ or casein kinase 1ε leads to a change in the interaction between p53 and mdm2. It is also known that casein kinase 1δ and casein kinase 1ε are involved as a regulatory protein associated with the formation of a spindle as a central body during cell division, and that casein kinase 1δ and casein kinase 1ε are involved in apoptosis mediated by TRAIL (tumor necrosis factor-related apoptosis inducing factor) and Fas. It has been further reported that inhibition of casein kinase 1δ or casein kinase ε by nonselective CK1 inhibitory compound IC261 reduces pancreatic tumor cell growth in vitro and in vivo (Brockschmidt et al., 2008, DOI: 10.1136/gut.2007.123695). Hence, CK1 inhibitors have been investigated for various important phenotypic and therapeutic effects.

WO2011051858 discloses CK1 inhibitors (both δ and ε) useful in the treatment and/or prevention of diseases and disorders associated with the central nervous system. These inhibitors form a series of substituted imidazole compounds, more specifically a series of 4-aryl-5-heteroaryl-1-heterocycloalkyl-imidazoles and related analogs. Both their synthesis and IC₅₀ values for CK1 δ and ε are reported, the latter of which generally fall in the nanomolar range. A closely related family of CK1 inhibitors is disclosed in WO2012085721.

WO2015119579 discloses a family that also features an azole core, namely a family of 2,4,5-tri-substituted azole compounds for use as CK1 inhibitors. The inhibitors are used for inducing or enhancing the differentiation of pluripotent stem cells into cardiomyocytes via CK1 inhibition. Synthetic pathways for obtaining the inhibitors are disclosed, and the inhibitors are shown to generally have IC₅₀ values in the nanomolar range as CK1 δ and ε inhibitors.

EP2949651 discloses a family of derivatives of substituted benzothiazoles that act as CK1 inhibitors, and their use is coupled to the treatment and/or prevention of diseases mediated by CK1, especially inflammatory, neurological, psychiatric, neurodegenerative and/or ophthalmic diseases and certain regenerative processes. Methods of synthesis are provided, and the inhibitors were shown to have nanomolar inhibitory activity on CK1 δ and ε. Bischof et al., Amino Acids (2012) 43:1577-1591 DOI: 10.1007/s00726-012-1234-x, Garcia-Reyes et al., J. Med. Chem. 2018, 61, 4087-4102, DOI: 10.102¹/ₐcs.jmedchem.8b00095, and Richter et al., J. Med. Chem., DOI: 10.1021/jm500600b describe similar CK1 inhibitors.

WO2009016286 discloses 6-cycloamino-3-(pyrid-4-yl)imidazo[1,2-b]pyridazine derivatives useful as protein kinase inhibitors, particularly as CK1 δ and ε inhibitors. Their synthesis is described in detail, and the capacity of the CK1 inhibitors to inhibit the phosphorylation of casein by casein kinases 1δ and ε was evaluated according to the procedure described in US2005/0131012, revealing IC₅₀ values in the nanomolar range.

WO2015195880 discloses a family with a similar core, namely substituted bicyclic pyrazoles useful as protein kinase inhibitors. Synthetic strategies for obtaining the inhibitors are described, and the resulting CK1 inhibitors were shown to be effective on CK1 δ and ε. A particular relevance is indicated for the treatment of cancer.

Hirota et al., PLoS Biot 8(12): e1000559. doi:10.1371/journal.pbio.1000559 and Monastyrskyi et al., Bioorg Med Chem. 2018 Feb 1;26(3):590-602. doi: 10.1016/j.bmc.2017.12.020 describe CK1 inhibitors that comprise a 6-aminopurine core, relevant for treatment of circadian rhythm disorders or of cancer.

Halekotte et al., Molecules 2017, DOI: 10.3390/molecu1es22040522 , Luxenburger et al., Molecules 2019, DOI: 10.3390/molecu1es24050873, and Peifer et al., J. Med. Chem. 2009, 52, 7618-7630 DOI: 10.1021/jm9005127 describe CK1 inhibitors with an azole or heteroazole core. Their use is linked to treating cancer, neurodegenerative diseases, sleeping disorders, or inflammation.

Members of the p38 MAPK family, composed of α, β, γ and δ, isoforms are encoded by separate genes that play a critical role in cellular responses needed for adaptation to stress and survival (reviewed in Whitmarsh 2010 DOI: 10.1186/1741-7007-8-47; Krementsov et al., 2013, DOI: 10.1128/MCB.00688-13). In many inflammatory diseases, including cardiovascular and other chronic diseases, these same p38 MAPK stress-induced signals can trigger maladaptive responses that aggravate, rather than alleviate, the disease (reviewed in Whitmarsh 2010). Indeed, in skeletal muscle, a variety of cellular stresses including chronic exercise, insulin exposure and altered endocrine states, myoblast differentiation into myocytes, reactive oxygen species, as well as apoptosis, have all been shown to induce the p38 kinase pathway (Keren, et. al., 2006, DOI: 10.1016/j.mce.2006.03.017; Zarubin et al., 2006, DOI: 10.1038/sj.cr.7290257). In fact, the p38 kinase pathway can be activated by a number of external stimuli, including pro-inflammatory cytokines and cellular stress, leading to activation of the dual-specificity MAPK kinases MKK3 and MKK6. Activation of MKK3 and MKK6, which in turn phosphorylate p38 in its activation loop, trigger downstream phosphorylation events. These include phosphorylation of HSP27, MAPKAPK2 (MK2) and a variety of transcription factors culminating in transcriptional changes in the nucleus. A modest number of p38-regulated transcripts and a large number of downstream effectors of p38 kinase have been identified (described in Cuenda et al., 2007, DOI: 10.1016/j.bbamcr.2007.03.010 and Kyriakis et. al., 2001, DOI: 10.1152/physrev.2001.81.2.807 , and Viemann et al. 2004, DOI: 10.1182/blood-2003-09-3296).

Several compounds from different chemical scaffolds that inhibit the p38α MAPK signaling pathway have entered clinical trials in diverse (non-neuromuscular) indications, including rheumatoid arthritis, chronic obstructive pulmonary disease, pain, cardiovascular diseases, and cancer.

p38α MAPK is known to play critical roles in skeletal muscle biology, specifically in abrogating proliferating myoblasts to differentiation and subsequently fusion to form multi-nucleated myotubes. Activation of this pathway is thought to be intrinsic to muscle cells expressing MyoD and serves to guarantee the complex and timely activation of the muscle program (Keren et al., 2006). Treatment of FSHD using p38 inhibitors is disclosed in WO2019103926. Treatment of muscular dystrophy patients that are constitutively undergoing processes of degeneration and regeneration with p38α inhibitors is disclosed in WO2019/071144 and WO2019/071147. Complete knockout (KO) of p38α is embryonically lethal. Embryonic rescue allows for survival of pups to a few days postnatal and isolation of satellite cells to study Myogenic precursors lacking p38α. Myoblasts lacking p38α express significantly less critical differentiation genes and show severe deficits in fusion. Histology of P2 pups show significantly increased cycling satellite cells and a left-shifted fiber distribution. (Perdiguero et. al, 2007, DOI: 10.1038/sj.emboj.7601587). The p38 pathway activation was shown to be enhanced in mdx and Sgcd^{-/-}mice, a genetic model of Duchenne muscular dystrophy or Limb-Girdle muscular dystrophy, respectively. In the mdx mice, knockout (KO) of p38α in mature muscle (cre driven by Myl1 promoter) showed no deficiencies in early time points, and mice deficient in p38α at 6 months of age showed significantly greater central nucleation and a smaller fiber distribution compared to controls. No other pathological indexes of disease were observed, such as fibrosis or increase in serum creatine kinase, suggesting that loss of p38α from skeletal muscle was not pathologic to mature fibers but was enhancing wild type satellite cell activity or was otherwise affecting the movement of central nuclei to the periphery of myofibers as they mature (Wissing et al., 2014, DOI: 10.1093/hmg/ddu270). The Myl1-cre knock-in allele that was employed to delete p38 in that study is not active in satellite cells, so the p38 loss cannot directly affect regeneration of skeletal muscle, indicating that the protection conferred by p38 loss should instead be ascribed to for example decreased myofiber necrosis without effects from satellite cells (Wissing et al., 2014). In primary FSHD muscle cells, p38 is not upregulated compared to cells from healthy donors, suggesting that potential effects of p38 inhibitors to reduce necrosis cannot necessarily be generalized to all muscular dystrophies, including FSHD. Taking this into account, while pharmacologic p38 inhibitors may be used to mitigate dystrophic pathology of muscular dystrophies where p38 activity is increased, even in those diseases such use is not mechanistically straightforward given the known antagonistic effects of p38 inhibitors on satellite cells and on muscle regeneration.

In light of the above, there is a need for compounds or combinations of compounds that inhibit DUX4 and/or the inflammatory response in FSHD without affecting myotube formation. There is a need for compounds that reduce inhibition of myotube formation. There is a need for compounds the reduce p38-inhibitor-induced impairment of myotube formation. There is a need for improved DUX4 reduction, preferably without affecting myotube formation. There is a need for promotion of myotube formation, preferably during treatment of FSHD. There is a need for improved effect of p38 inhibitors in treatment of DUX4-related conditions, such as in FSHD-treatment. There is a need for improved treatment of subjects suffering from DUX4-related conditions such as FSHD, who also suffer from inflammation.

### SUMMARY OF THE INVENTION

In a first aspect the invention pertains to a casein kinase 1 inhibitor for use in the treatment of a disease or condition associated with DUX4 expression in a subject, wherein preferably, the subject suffers from muscle inflammation. Preferably, the casein kinase 1 inhibitor is used for promoting at least one of myogenic fusion and differentiation, wherein preferably, the subject suffers from muscle inflammation. Preferably, said disease or condition associated with DUX4 expression is a muscular dystrophy or cancer, of which a muscular dystrophy is more preferred. Most preferably, said disease or condition associated with DUX4 expression is facioscapulohumeral muscular dystrophy (FSHD). Preferably, a casein kinase 1 inhibitor for use in accordance with the invention reduces DUX4 expression by at least 20%, 40%, 60%, 80%, or more. Preferably, the casein kinase inhibitor inhibits at least casein kinase 1δ.

In a second aspect the invention relates to a combination of a casein kinase 1 inhibitor and a p38 inhibitor for use in the treatment of a disease or condition associated with DUX4 expression in a subject, wherein preferably, the subject suffers from muscle inflammation. Preferably, the casein kinase 1 inhibitor is used for promoting at least one of myogenic fusion and differentiation, wherein preferably, the subject suffers from muscle inflammation. The casein kinase 1 inhibitor and the p38 inhibitor can either be two distinct substances, or the casein kinase 1 inhibitor and the p38 inhibitor can be one and the same substance. Preferably, said disease or condition associated with DUX4 expression is a muscular dystrophy or cancer, of which a muscular dystrophy is more preferred. Most preferably, said disease or condition associated with DUX4 expression is facioscapulohumeral muscular dystrophy (FSHD). Preferably, the p38 inhibitor inhibits at least p38α. Preferably, the casein kinase inhibitor inhibits at least casein kinase 1δ. Preferably, a combination of a casein kinase 1 inhibitor and a p38 inhibitor for use in accordance with the invention reduces DUX4 expression by at least 20%, 40%, 60%, 80%, or more.

In a third aspect. the invention pertains to an in vivo, in vitro, or ex vivo method for promoting myogenic fusion and/or differentiation, the method comprising the step of contacting a cell with a casein kinase 1 inhibitor as defined herein, or with a combination as defined herein.

In a fourth aspect, the invention relates to a method for reducing DUX4 expression in a subject in need thereof, the method comprising the step of administering an effective amount of a casein kinase 1 inhibitor as defined herein, or the step of administering an effective amount of a combination as defined herein, wherein the subject suffers from muscle inflammation. Preferably, the subject suffers from a disease or condition associated with DUX4 expression that is a muscular dystrophy or cancer, of which a muscular dystrophy is more preferred. Most preferably, said muscular dystrophy associated with DUX4 expression is facioscapulohumeral muscular dystrophy (FSHD).

### DESCRIPTION OF EMBODIMENTS

The inventors have surprisingly found that CK1 inhibitors improve myogenic fusion. This promotes myotube formation. This effect is especially useful when other drugs for treating DUX4-related conditions are administered, such as when p38 inhibitors are administered, because such drugs impair myotube formation. To overcome this undesirable iatrogenic phenomenon, CK1 inhibitors can be coadministered. For example, a combination of a CK1 inhibitor and a p38 inhibitor was found to reduce DUX4 expression while promoting correct myotube formation. Accordingly the invention provides the use of a CK1 inhibitor to improve myogenic fusion in a subject with iatrogenic impaired myotube formation, e.g. as the result of the administration of a p38 inhibitor

The inventors have identified the inflammatory response to fiber damage as a compelling candidate mechanism for exacerbation of DUX4-mediated conditions such as FSHD. The dysregulated expression of an embryonic transcription factor in muscle cells induces a variety of molecules that can function as neoantigens that are directly recognized by the infiltrating T cells. In fact, some of the genes regulated by DUX4, such as the PRAME family, are known cancer testis antigens, so expression of these genes in skeletal muscle can also induce an adaptive immune response. In addition, the induction of DEFB103 by DUX4 influences both the adaptive and the innate immune response. DEFB103 can have a proinflammatory role in the adaptive immune response and can act as a chemo-attractant for monocytes, lymphocytes, and dendritic cells. In this regard, it can enhance an adaptive immune response to germline antigens expressed in FSHD muscle (Geng et al. 2012, DOI: 10.1016/j.devce1.2011.11.013). Finally, the effector mechanisms deployed by the immune cells on the muscle tissue, such as but not limited to cytokines and inflammatory factors that are released during the disease process, promote deleterious signaling events that directly participate in myofiber death, and can act as a trigger for DUX4 expression. Taken together, inflammatory markers, such as for example STIR MRI imaging have substantial predictive value for identifying patients with inflammation and DUX4 expression, both of which markers characterize active disease. According the invention provides for compounds and combinations of compounds for treating subjects having been diagnosed with active FSHD.

Following the central role of DUX4 in the consensus disease hypothesis for FSHD, a therapeutic approach with a disease-modifying potential is expected to rely on the inhibition of DUX4. The inventors have surprisingly identified Caseine kinase 1 (CK1) as a novel drug target to achieve DUX4 repression in muscle cells, and to promote myogenic fusion in muscle cells, particularly in muscle cells with impaired myogenic fusion. This invention has been made using primary FSHD patient-derived muscle cells. Because of the primate-specificity of the FSHD locus and questionable relevance of recombinant, immortalized, or tumorigenic cell or animal models to study endogenous DUX4 regulatory mechanisms, primary patient-derived muscle cells are the most relevant disease model available. Assays based on immortalized cells bear the risk of altered epigenomes, thereby limiting their relevance in studying the endogenous regulation of DUX4 expression. Particularly the subtelomeric location of D4Z4 and the importance of the D4Z4 epigenome in the stability of DUX4 repression (Stadler et al., 2013, DOI: 10.1038/nsmb.2571) underscore the necessity of using primary muscle cells to discover physiologically relevant drug targets that regulate the expression of DUX4.

DUX4 has historically been regarded as being challenging to detect in FSHD muscle. Its expression in primary myoblasts from patients with FSHD has been shown to be stochastic. Studies have reported that only 1 in 1000 or 1 in 200 nuclei is DUX4 positive in proliferating FSHD myoblasts and during myoblast differentiation, respectively. Due to this particularly low abundance of DUX4, detection of DUX4 protein has been reported to be a technical challenge. While primary FSHD muscle cells have been used extensively in the FSHD literature, none of the reports appear to be applicable beyond a bench scale level. The limitations posed by using primary cells and the recognised complexity of detecting the low levels of endogenous DUX4 illustrate the challenges associated with applying primary FSHD muscle cells to higher throughput formats. Although DUX4 expression increases upon in vitro differentiation of proliferating FSHD myoblasts into multinucleated myotubes, the levels remain low and the dynamic variability is widely accepted to be extremely challenging for robust large-scale screening approaches (Campbell et al., 2017).

### Compound for Use

In a first aspect the invention provides a casein kinase 1 (CK1) inhibitor for use in the treatment of a disease or condition associated with (undue) DUX4 expression, wherein the casein kinase 1 inhibitor reduces DUX4 expression. Such a CK1 inhibitor is referred to herein as a CK1 inhibitor for use according to the invention. CK1 inhibitors are known in the art and are described in more detail later herein.

The medical use herein described is formulated as a compound as defined herein for use as a medicament for treatment of the stated condition(s) (e.g. by administration of an effective amount of the compound), but could equally be formulated as i) a method of treatment of the stated condition(s) using a compound as defined herein comprising a step of administering to a subject an effective amount of the compound, ii) a compound as defined herein for use in the manufacture of a medicament to treat the stated condition(s), wherein preferably the compound is to be administered in an effective amount, and iii) use of a compound as defined herein for the treatment of the stated condition(s), preferably by administering an effective amount. Such medical uses are all envisaged by the present invention. Preferred subjects are subjects in need of treatment. Treatment preferably leads to delay, amelioration, alleviation, stabilization, cure, or prevention of a disease or condition. In other words, a compound for use according to the invention can be a compound for the treatment, delay, amelioration, alleviation, stabilization, cure, or prevention of the stated disease or condition. Particularly, compounds for use according to the invention can be for ameliorating detrimental side effects of other medicaments.

The CK1 inhibitor for use according to the invention reduces DUX4 expression. This DUX4 expression is preferably the overall DUX4 expression of the subject that is treated. DUX4 expression can be determined using methods known in the art, or exemplified in the examples. For example, DUX4 expression can be determined using PCR techniques such as RT-PCR, or using immunostaining, mass spectrometry, or ELISA, for example on a sample containing cells or cell extracts, preferably obtained from the subject. In this context, a reduction is preferably a reduction as compared to either a predetermined value, or to a reference value. A preferred reference value is a reference value obtained by determining DUX4 expression in an untreated sample containing cells or cell extracts. This untreated sample can be from the same subject or from a different and healthy subject, more preferably it is a sample that was obtained in the same way, thus containing the same type of cells. Conveniently, both the test sample and the reference sample can be part of a single larger sample that was obtained. Alternately, the test sample was obtained from the subject before treatment commenced. A highly preferred reference value is the expression level of DUX4 in a sample obtained from a subject prior to the first administration of the casein kinase 1 inhibitor according to the invention. Another preferred reference value is a fixed value that represents an absence of DUX4 expression.

A reduction of DUX4 expression preferably means that expression is reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. If expression of DUX4 is reduced by for example 100%, it may be that expression of DUX4 can no longer be detected. Reduction can be assessed at the protein level, for example through immunostaining, ELISA, or mass spectrometry, or it can be assessed at the mRNA level, for example through PCR techniques such as RT-PCR. In preferred embodiments, the invention provides a casein kinase 1 inhibitor for use according to the invention, wherein the reduction of DUX4 expression is determined using PCR or immunostaining, wherein a preferred PCR technique is RT-PCR. In preferred embodiments the invention provides a casein kinase 1 inhibitor for use according to the invention, wherein DUX4 expression is reduced by at least 20%, 40%, 60%, 80%, or more, more preferably by at least 30%, 40%, 60%, 80%, or more. In further preferred embodiments, DUX4 expression is reduced by at least 10%. In further preferred embodiments, DUX4 expression is reduced by at least 20%. In further preferred embodiments, DUX4 expression is reduced by at least 30%. In further preferred embodiments, DUX4 expression is reduced by at least 40%. In further preferred embodiments, DUX4 expression is reduced by at least 50%. In further preferred embodiments, DUX4 expression is reduced by at least 60%. In further preferred embodiments, DUX4 expression is reduced by at least 70%. In further preferred embodiments, DUX4 expression is reduced by at least 80%. In further preferred embodiments, DUX4 expression is reduced by at least 90%. In further preferred embodiments, DUX4 expression is reduced by at least 95%. In the most preferred embodiments, DUX4 expression is reduced by about 100%, preferably by 100%.

In preferred embodiments, the invention provides a casein kinase 1 inhibitor for use according to the invention, wherein the casein kinase 1 inhibitor reduces DUX4 expression in muscle cells, immune cells, or cancer cells, preferably in muscle cells or immune cells, most preferably in muscle cells. Preferred muscle cells are myoblasts, satellite cells, myotubes, and myofibers. Preferred immune cells are B cells, T cells, dendritic cells, neutrophils, natural killer cells, granulocytes, innate lymphoid cells, megakaryocytes, myeloid-derived suppressor cells, monocytes/macrophages, and thymocytes, and optionally mast cells. Other preferred cells are platelets and red blood cells. In other embodiments, DUX4 expression is reduced in cancer cells.

In preferred embodiments the invention provides the CK1 inhibitors for use according to the invention, wherein said disease or condition associated with DUX4 expression is a muscular dystrophy or cancer, preferably wherein said disease or condition associated with DUX4 expression is a muscular dystrophy, most preferably facioscapulohumeral muscular dystrophy (FSHD).

In this context, a preferred muscular dystrophy is FSHD; a preferred cancer is prostate cancer (WO2014081923), multiple myeloma (US20140221313), lung cancer (Lang et al., 2014, DOI: 10.14205/2310-8703.2014.02.01.1), colon cancer (Paz et al., 2003, DOI: 10.1093/hmg/ddg226) sarcoma, or leukemia; a preferred sarcoma is small round cell sarcoma (Oyama et al., 2017 DOI: 10.1038/s41598-017-04967-0 ; Bergerat et al., 2017, DOI: 10.1016/j.prp.2016.11.015 ; Chebib and Jo, 2016, DOI: 10.1002/cncy.21685); a preferred leukemia is acute lymphoblastic leukemia (ALL), more particularly B-cell precursor ALL (Yasuda et al., 2016, doi: 10.1038/ng.3535 ; Lilljebjörn & Fioretos, 2017, DOI: 10.1182/blood-2017-05-742643 ; Zhang et al., 2017, DOI:10.1038/ng.3691).

Accordingly, in preferred embodiments, the invention provides the CK1 inhibitors for use according to the invention, wherein said disease or condition associated with DUX4 expression is a muscular dystrophy or cancer, preferably wherein said disease or condition associated with DUX4 expression is FSHD, prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), preferably said disease or condition associated with DUX4 expression is FSHD. In more preferred embodiments, the invention provides the CK1 inhibitors for use according to the invention, wherein said disease or condition associated with DUX4 expression is a muscular dystrophy or cancer, preferably wherein said disease or condition associated with DUX4 expression is FSHD or cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

In a preferred embodiment, the invention provides the CK1 inhibitors for use according to the invention, wherein said disease or condition associated with DUX4 expression is cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

Other DUX4 targets are known as “cancer testis antigens” (CTAs), which are genes that are normally expressed only in testis, but which are de-repressed in some cancers, eliciting an immune response. These observations imply that DUX4 de-repression in cancers mediates the activation of HSATII, CTAs and/or THE1B promoters (Young et al., 2013, doi:10.1371/journal.pgen.1003947). In line with this, Dmitriev et al. (2014, DOI: 10.1111/jcmm.12182) demonstrate a similarity between FSHD and cancer cell expression profiles, suggesting a common step in the pathogenesis of these diseases.

### Inhibitor Combinations

In an aspect, the invention provides combinations of a CK1 inhibitor and an agent that is known to impair myogenic fusion and/or differentiation. The agent that is known to impair myogenic fusion and/or to impair myogenic differentiation generally does so as a side effect, for example impaired myogenic fusion can be an iatrogenic phenomenon associated with the agent. Such a combination is beneficial because the inventors have found that inhibition of CK1 promotes myogenic fusion and differentiation, and ameliorates the impairing effect on myogenic fusion or differentiation that the other agent has. This allows both the CK1 inhibitor and the other agent to be efficaciously used in the treatment of muscular dystrophies such as preferably FSHD. Such a combination is referred to herein as a combination according to the invention.

Preferably, the agent that impairs myogenic fusion and/or differentiation is selected from the group consisting of a p38 inhibitor, a β₂ adrenergic receptor agonist and a BET inhibitor. Most preferably the agent that impairs myogenic fusion and/or differentiation is a p38 inhibitor; such inhibitors are defined later herein. In other preferred embodiments, the agent that impairs myogenic fusion and/or differentiation is a β₂ adrenergic receptor agonist, more preferably selected from the group consisting of abediterol, alifedrine, amibegron, arbutamine, arformoterol, arotinolol, BAAM, bambuterol, befunolol, bitolterol , broxaterol, buphenine, carbuterol, carmoterol, cimaterol, clenbuterol, colterol, corbadrine, denopamine, dipivefrine, dobutamine, edopamine, dopexamine, droxidopa (L-DOPS), ephedrine, epinephrine, etafedrine, etilefrine, etilevodopa, ethylnorepinephrine, fenoterol, formoterol, hexoprenaline, higenamine, indacaterol, isoetarine, isoprenaline, isoxsuprine, L-DOPA (levodopa), L-phenylalanine, L-tyrosine, levosalbutamol, mabuterol, melevodopa, methoxyphenamine, methyldopa, mirabegron, norepinephrine, orciprenaline, oxyfedrine, PF-610355, phenylpropanolamine, pirbuterol, prenalterol, ractopamine, procaterol, pseudoephedrine, reproterol, rimiterol, ritodrine, salbutamol, salmeterol, solabegron, terbutaline, tretoquinol, tulobuterol, vilanteroleamoterol, XP21279, zilpaterol, and zinterol, more preferably from salbutamol, terbutaline, salmeterol, tulobuterol, and bambuterol.

In preferred embodiments, a combination according to the invention is a combination comprising at least two distinct compounds, a first compound being a CK1 inhibitor, and a second compound being the second agent. A preferred example of such a combination is a combination comprising a CK1 inhibitor and comprising a further compound that is a p38 inhibitor. Another preferred example of such a combination is a combination comprising a CK1 inhibitor and comprising a further compound that is a β₂ adrenergic receptor agonist. Such a combination is referred to hereinafter as a two-compound combination according to the invention.

Preferred two-compound combinations according to the invention comprise molar ratios of inhibitors as shown in the below table, wherein “further” indicates the second compound. Preferred two-compound combinations according to the invention comprise weight ratios of inhibitors as shown in the below table. Preferred two-compound combinations according to the invention comprise inhibitory activity ratios for inhibitors as shown in the below table.

| Entry | CK1 | Further |
|---|---|---|
| 1 | 10% | 90% |
| 2 | 20% | 80% |
| 3 | 30% | 70% |
| 4 | 40% | 60% |
| 5 | 50% | 50% |
| 6 | 60% | 40% |
| 7 | 70% | 30% |
| 8 | 80% | 20% |
| 9 | 90% | 10% |
| 10 | 95% | 5% |
| 11 | 20-80% | 80-20% |
| 12 | 30-70% | 70-30% |
| 13 | 40-60% | 60-40% |
| 14 | 45-65% | 65-45% |
| 15 | 10-90% | 90-10% |

Preferred two-compound combinations according to the invention comprise an amount of CK1 inhibitor effective to inhibit at least 5, 10, 15, 20, 25, 30, 35, 40, 45, 50, 55, 60, 65, 70, 75, 80, 85, 90, 95, 96, 97, 98, 99, or 100% of CK1 activity in a subject or in a sample, more preferably at least 40%, even more preferably at least 60%, most preferably at least 80%. Preferred two-compound combinations according to the invention comprise an amount of p38 inhibitor effective to inhibit at least 5, 10, 15, 20, 25, 30, 35, 40, 45, 50, 55, 60, 65, 70, 75, 80, 85, 90, 95, 96, 97, 98, 99, or 100% of p38 activity in a subject or in a sample, more preferably at least 40%, even more preferably at least 60%, most preferably at least 80%. In preferred two-compound combinations according to the invention, the further agent is a p38 inhibitor, preferably losmapimod.

In preferred embodiments, a combination according to the invention comprises a CK1 inhibitor that has an additional kinase inhibitory function that would independently impair myogenic fusion and/or differentiation. A preferred example thereof is a CK1 inhibitor that is also a p38 inhibitor. Such a combination is referred to hereinafter as a functional combination according to the invention.

Preferred combinations according to the invention (either two-compound or functional) inhibit CK1 and p38. Preferably, activity of CK1 is inhibited by least 5, 10, 15, 20, 25, 30, 35, 40, 45, 50, 55, 60, 65, 70, 75, 80, 85, 90, 95, 96, 97, 98, 99, or 100%. Preferably, activity of p38 is inhibited by at least 5, 10, 15, 20, 25, 30, 35, 40, 45, 50, 55, 60, 65, 70, 75, 80, 85, 90, 95, 96, 97, 98, 99, or 100%. More preferably, both CK1 and p38 are inhibited by at least 5%, more preferably by at least 10%, even more preferably by at least 15%, more preferably still by at least 20%, more preferably by at least 25%, even more preferably by at least 30%, still more preferably by at least 35%, more preferably still by at least 40%, even more preferably by at least 45%, more preferably by at least 50%, more preferably still be at least 55%, even more preferably by at least 60%, more preferably still by at least 65%, even more preferably by at least 70%, more preferably by at least 75%, more preferably by at least 80%, even more preferably by at least 85%, even more preferably by at least 90%, most preferably by at least 95, 96, 97, 98, 99, or 100%.

### Promotion of Myogenic Fusion and/or Differentiation

In preferred embodiments, a CK1 inhibitor as defined herein or a combination as defined herein is for the promotion of myogenic fusion and/or for the promotion of myogenic differentiation. The inventors have identified that CK1 inhibitors promote both of these important characteristics of healthy or recovering muscles. The use in promoting myogenic fusion and/or myogenic differentiation aids with muscle regeneration.

Myogenic fusion is the formation of multi-nucleated myotubes from a plurality of myoblasts. A myoblast is a type of embryonic progenitor cell that differentiates to give rise to muscle cells. Differentiation is regulated by myogenic regulatory factors, including MyoD, Myf5, myogenin, and MRF4. GATA4 and GATA6 also play a role in myocyte differentiation. Skeletal muscle fibers are made when myoblasts fuse together; muscle fibers therefore are cells with multiple nuclei, known as myonuclei, with each cell nucleus originating from a single myoblast. The fusion of myoblasts is specific to skeletal muscle (e.g., biceps brachii) and not cardiac muscle or smooth muscle. Myoblasts in skeletal muscle that do not form muscle fibers dedifferentiate back into myosatellite cells. These satellite cells remain adjacent to a skeletal muscle fiber, situated between the sarcolemma and the basement membrane of the endomysium (the connective tissue investment that divides the muscle fascicles into individual fibers). To re-activate myogenesis, the satellite cells must be stimulated to differentiate into new fibers. The inventors have identified that CK1 inhibitors promote this differentiation of satellite cells, thus ultimately promoting myotube formation and myogenesis.

p38 inhibitors are known to impair normal functioning of skeletal muscle biology, specifically in impairing proliferating myoblasts to undergo differentiation and to subsequently fuse to form multi-nucleated myotubes. The invention provides a CK1 inhibitor for use in the treatment of a disease or condition associated with DUX4 expression in a subject, wherein the CK1 inhibitor is for promoting myogenic fusion and/or differentiation. Such promoted fusion and differentiation helps reinstate healthy skeletal muscle biology. Preferably, the subject also suffers from muscle inflammation; this enables the improved use as described elsewhere herein.

In preferred embodiments, the CK1 inhibitor is for promoting myogenic fusion. Myogenic fusion is quintessential to muscle formation and muscle regeneration, and it can be assessed using any known method. Preferably, it is assessed using image analysis, more preferably using high content image analysis, most preferably as described in the examples. In preferred embodiments, the CK1 inhibitor for promoting myogenic fusion increases myogenic fusion with at least 1, 2, 3, 4, 5, 6, 7, 8, 9, 10, 11, 12 ,13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic fusion was present in a subject or in a muscle or in a sample. In such a case the CK1 inhibitor for promoting myogenic fusion preferably reinstates myogenic fusion, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments the CK1 inhibitor is for promoting myogenic differentiation. In these embodiments, a cell is preferably a primary cell. In these embodiments, a cell is preferably not an immortalized cell. Myogenic differentiation can be assessed using methods known in the art, such as quantification of myogenic differentiation markers such as MYH2, MyoD, Myf5, myogenin, and MRF4, preferably such as myogenin or MYH2. In preferred embodiments, the CK1 inhibitor for promoting myogenic differentiation increases myogenic differentiation with at least 1, 2, 3, 4, 5, 6, 7, 8, 9, 10, 11, 12 ,13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic differentiation was present in a subject or in a muscle or in a sample. In such a case the CK1 inhibitor for promoting myogenic differentiation preferably reinstates myogenic differentiation, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments, the combination according to the invention is for promoting myogenic fusion, wherein features and definitions are as defined elsewhere herein. In preferred embodiments the combination according to the invention is for promoting myogenic differentiation, wherein features and definitions are as defined elsewhere herein. In preferred embodiments, the combination according to the invention is for promoting myogenic fusion and/or differentiation, wherein features and definitions are as defined elsewhere herein. Use of the combination is especially beneficial because the activity of the CK1 inhibitor mitigates the detrimental side effects of the further agent, such as of the p38 inhibitor. When for use as described in this section, a combination according to the invention preferably comprises a p38 inhibitor as the further agent. Such a p38 inhibitor is known to impair myogenic fusion and/or differentiation. The inventors have found a surprising synergistic effect on DUX4 reduction, and have identified CK1 inhibitors as suitable for reducing the undesirable side effects of p38 inhibitors on muscle cells.

### Treatment of Dystrophy Patients Suffering From Muscle Inflammation

In preferred embodiments, a CK1 inhibitor as defined herein or a combination as defined herein is for the the treatment of patients suffering from both a DUX4-related condition and from muscle inflammation. Muscle inflammation contributes to the pathophysiology of muscular dystrophies such as FSHD. It precedes muscle destruction and fatty replacement, thereby representing an early marker for disease activity. Muscle inflammation can be identified using means known in the art. Preferably, muscle inflammation is identified by at least one of using biopsies and using MRI sequences with short TI inversion recovery (STIR), preferably using MRI with STIR. STIR hyperintensities (STIR+) visualize edema, which correlates with inflammation. A preferred inflamed muscle is a STIR+ muscle. A preferred muscle biopsie is a biopsie from a STIR+ muscle. A preferred muscle inflammation is MAPK-associated muscle inflammation, more preferably a muscle inflammation associated with the transcription and translation of inflammatory response-associated genes that encode proteins such as TNF-a, IL-1b, IL-6, and IL-8. Muscle inflammation predicts a faster fat replacement of muscle. p38 inhibitors are known anti-inflammatory agents, but reduce myogenic fusion and impair myogenic differentiation and regeneration. A combination of a CK1 inhibitor and a p38 inhibitor overcomes these problems and results in a synergistic reduction of DUX4 expression.

A preferred subject suffering from muscle inflammation has at least one inflamed muscle, more preferably at least 2, even more preferably at least 3, even more preferably at least 4, even more preferably at least 5, most preferably at least 6, 7, 8, 9, 10, or 11. Preferably the inflamed muscle is a skeletal muscle, more preferably it is a skeletal muscle of the face, scapula, or upper arms. A preferred subject suffering from muscle inflammation is a subject also suffering from muscular dystrophy, more preferably also suffering from FSHD. Preferably, such a subject suffering from FSHD has at least one inflamed muscle, more preferably at least one STIR+ muscle.

The invention provides a casein kinase 1 inhibitor for use in the treatment of a disease or condition associated with DUX4 expression in a subject, wherein the subject suffers from muscle inflammation. In preferred embodiments, the invention provides a casein kinase 1 inhibitor for use in the treatment of FSHD, wherein the subject suffers from muscle inflammation. In preferred embodiments, the invention provides a casein kinase 1 inhibitor for use in the treatment of FSHD, wherein the subject has at least one inflamed muscle, preferably at least one inflamed skeletal muscle of the face, scapula, or upper arms. This muscle is preferably STIR+. Muscle inflammation is known to precede fatty infiltration. Accordingly, the invention provides a casein kinase 1 inhibitor for preventing or delaying fatty infiltration in a muscle of a subject suffering from FSHD.

The invention provides a combination according to the invention for use in the treatment of a disease or condition associated with DUX4 expression in a subject, wherein the subject suffers from muscle inflammation as herein defined. In preferred embodiments, the invention provides a combination according to the invention for use in the treatment of FSHD, wherein the subject suffers from muscle inflammation. In preferred embodiments, the invention provides a a combination according to the invention for use in the treatment of FSHD, wherein the subject has at least one inflamed muscle, preferably at least one inflamed skeletal muscle of the face, scapula, or upper arms. This muscle is preferably STIR+. Muscle inflammation is known to precede fatty infiltration. Accordingly, the invention provides a combination according to the invention for preventing or delaying fatty infiltration in a muscle of a subject suffering from FSHD.

When for use as described in this section, a combination according to the invention preferably comprises a p38 inhibitor as the further agent. Such a p38 inhibitor is known to have an anti-inflammatory activity. The inventors have found a surprising synergistic effect on DUX4 reduction, and have identified CK1 inhibitors as suitable for reducing the undesirable side effects of p38 inhibitors on muscle cells.

### Casein Kinase 1 Inhibitor

Casein kinase 1 inhibitors (CK1 inhibitors) are known in the art. Preferred CK1 inhibitors are small molecules. Preferably, in the context of this invention, a casein kinase 1 inhibitor is of general structural formula (1a), (1b), (2a), (2b), (3a), (3b), or (4):

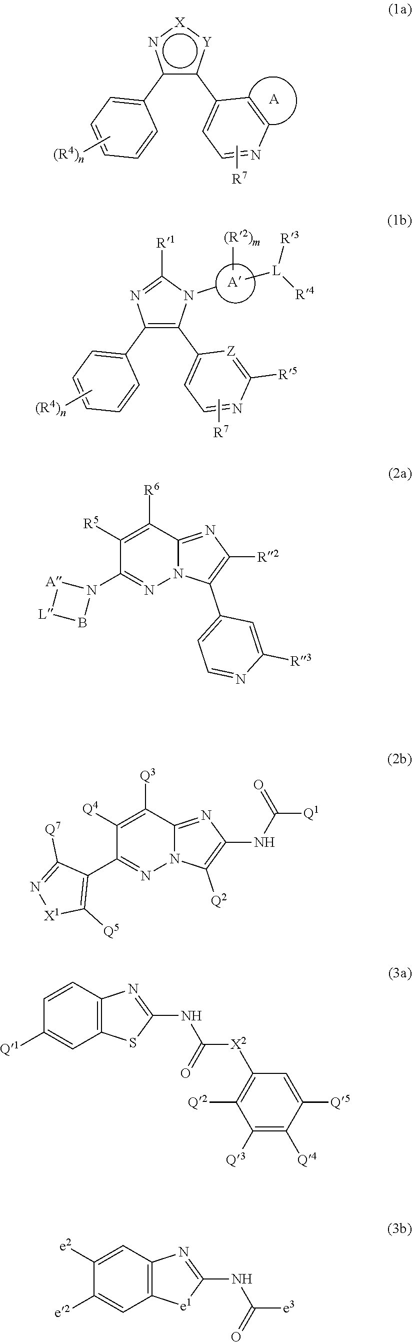

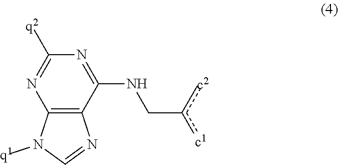

wherein X and Y are independently ═N—, —NR¹—, CR¹, —O—, or —S—, provided that at least one of X and Y is CR¹,

ring A is absent (so effectively it is two H) or is a 4- to 7-membered cycloalkyl or heterocycloalkyl or a 5- to 6-membered heteroaryl, wherein up to 2 carbon atoms are replaced with a heteroatom selected from ═N—, —NR²—, —O—, —S— and any remaining carbon atom may be substituted with R³ as valency allows; preferably, ring A is a 4- to 7-membered cycloalkyl or heterocycloalkyl or a 5- to 6-membered heteroaryl, wherein up to 2 carbon atoms are replaced with a heteroatom selected from ═N—, —NR²—, —O—, —S— and any remaining carbon atom may be substituted with R³ as valency allows;

each R¹ is independently H, C₁₋₄alkyl, C₃₋₆cycloalkyl, —CF₃, —(CH₂)₁₋₃CF₃, 4- to 10-membered aryl, 4- to 10-membered heteroaryl, 4- to 10-membered heterocycloalkyl, wherein said aryl, heteroaryl, or heterocycloalkyl may be substituted with one, two, or three substituents independently selected from halogen, OH, oxo, cyano, —SO₂CH₃, carboxylic acid that is optionally esterified with methanol or ethanol, carboxamide, nitro, C₁₋₆alkoxy, C₁₋₆alkyl, or C₁₋₆alkyl-O-C₁₋₆alkyl; preferably, each R¹ is independently H, C₁₋₄alkyl, C₃₋₆cycloalkyl, —CF₃, —(CH₂)₁₋₃—CF₃, 4- to 10-membered heterocycloalkyl, wherein said heterocycloalkyl may be substituted with up to two substituents independently selected from halogen, OH, oxo, cyano, C₁₋₆alkyl, or C₁₋₆alkyl-O—C₁₋₆alkyl; wherein when X is CR¹ the R¹ in that CR¹ moiety can also be —S—(CH₂)₀₋₃CH₃ or —(S═O)—(CH₂)₀₋₃CH₃;

Each R² is independently H, C₁₋₆alkyl, C₄₋₁₀-bicycloalkyl, —(CH₂)ₜ—CN, —SO₂—C₁₋₆alkyl, —SO₂(CH₂)ₜC₃₋₆cycloalkyl, —C₁₋₆alkyl-O—C₁₋₆alkyl, —C₁₋₆alkyl-C(O)O-C₁₋₆alkyl, —C₃₋₆cycloalkyl-C(O)O-C₁₋₆alkyl, —C(O)—(O)ᵤ—C₁₋₆alkyl, —C(O)—C₁₋₆alkyl-O—C₁₋₆alkyl, —C(O)—(O)ᵤ—(CH₂)ₜ—(CH₆₋₁₀aryl), —(CH₂)ₜ—(C₆₋₁₀aryl), —C(O)—(O)ᵤ'(CH₂)ₜ-(5- to 10-membered heteroaryl), —(CH₂)ₜ—C(O)—NR⁵R⁶, —(CH₂)ₜ-(5- to 10-membered heteroaryl), —C(O)—(O)ᵤ—(CH₂)ₜ-(3- to 10-membered heterocycloalkyl), -(CH₂)ₜ-(4- to 10-membered heterocycloalkyl), -C(O)-(O)ᵤ—(CH₂)ₜ-(3- to 10-membered cycloalkyl), or-(CH₂)ₜ-(3- to 10-membered cycloalkyl),

wherein said aryl, heteroaryl, cycloalkyl, and heterocycloalkyl of R² may be substituted with up to two substituents independently selected from halogen, OH, cyano, C₁₋₆alkyl, or C₁₋₆alkyl-O-C₁₋₆alkyl,

and wherein any alkyl, cycloalkyl, and heterocycloalkyl of R² may be further substituted with oxo where valency allows;

each R³ is independently absent, C₁₋₃alkyl, halogen, oxo, —NR⁵R⁶, or-OR⁵;

each R⁴ is independently halogen, —CF₃, C₁₋₃alkyl, —(CH₂)ₜ—C₃₋₄cycloalkyl, —(CH₂)ₜ—O—C₁₋₃alkyl, —(CH₂)ₜ-cyano, or-(CH₂)ₜ-hydroxy, wherein a halogen is preferably F and is preferably para to the five-membered ring comprising X and Y, wherein C₁₋₃alkyl is preferably methyl and is preferably meta to the five-membered ring comprising X and Y; preferably, each R⁴ is independently halogen, —CF₃, —(CH₂)ₜ—C₃₋₄cycloalkyl, —(CH₂)ₜ—O—C₁₋₃alkyl, —(CH₂)ₜ-cyano, or-(CH₂)ₜ-hydroxy;

each R⁵ is independently H or C₁₋₆alkyl;

each R⁶ is independently H or C₁₋₆alkyl;
or when the compound is of general formula (2a), R⁵ and R⁶ can, together with the carbon atoms to which they are attached, form —S—or —O—;

R⁷ is H, halogen, or C₁₋₃alkyl, or —Nr⁷r′⁷; wherein when R⁷ is —Nr⁷r′⁷ it is preferably ortho to the nitrogen in the pyridinyl moiety to which it is attached;

n is 0, 1, or 2; preferably 1;

each t is independently 0, 1, or 2;

each u is independently 0 or 1;

r⁷ and r′⁷ are each independently H or —(C═O)₀₋₁(NH)₀₋₁(CH₂)₀₋₄O₀₋₁r⁸; or r⁷ and r′⁷ together with the nitrogen to which they are attached form a cyclic structure; preferably at least one of r⁷ and r′⁷ is H; preferably the cyclic structure formed by r⁷ and r′⁷ is 5, 6, or 7-membered, more preferably it is piperazin-1-yl or 4-(R⁵)piperazin-1-yl or 4-phenylpiperazin-1-yl; wherein in preferred embodiments r⁷ is H and r′⁷ is r⁸;

r⁸ is H, C₁₋₆alkyl, substituted C₁₋₆alkyl, C₁₋₆acyl, substituted C₁₋₆acyl, cyclohexyl, cyclohexyl with 1 or 2 heteroatom substitutions, phenyl, substituted phenyl, naphthyl, pyrrolyl, substituted pyrrolyl, indolyl, substituted indolyl, or NR⁵R⁶, wherein acyl or alkyl moieties in r⁸ are optionally unsaturated, wherein substitutions in r⁸ are preferably selected from halogen, trifluoromethyl, methyl, ethyl, phenyl, fluorophenyl, methoxyphenyl, dimethoxyphenyl, methoxy, ethoxy, propyloxy, and phenoxy; preferably r⁸ is methoxyphenyl, dimethoxyphenyl, ethoxyphenyl, indolyl, dimethylamine, toluyl, chlorotoluyl, trifluoromethylphenyl, naphthyl, 1-methylpyrrol-2-yl, and 4-dimethoxyphenyl-1-methylpyrrol-2-yl; wherein more preferably r⁸ is C₁₋₆alkyl such as but-2-yl, oxanyl such as oxan-4-yl, 1-phenyleth-1-yl, acetyl, p-fluorophenylacetyl, 2-phenoxypropionyl, or 3-(o,p-dimethoxyphenyl)-propenoyl, wherein when r₈ is substituted pyrrolyl it is preferably 1-(r⁹)-4-(r¹⁰)-5-)r¹¹)-pyrrol-2-yl;

r⁹ is R⁵ or —(CH₂)₀₋₂pyrrolidinyl substituted with 0, 1, 2, or 3 hydroxy groups and 0 or 1 r′¹¹ moieties, preferably r⁹ is methyl or —CH₂-(3,4-dihydroxypyrrolidin-2-yl) or —CH₂-(3,4-dihydroxy-5-r′¹¹-pyrrolidin-2-yl);

r¹⁰ is H or phenyl or methoxylated phenyl, wherein methoxylated phenyl is preferably p-methoxyphenyl or o,p-dimethoxyphenyl;

r¹¹ is H or is linked to r′¹¹ by a —CH₂— or a —CH₂CH₂—moiety, preferably by a —CH₂— moiety;

r′¹¹ is H or is linked to r¹¹ by a —CH₂— or a —CH₂CH₂— moiety, preferably by a —CH₂— moiety; and wherein

A′ is a 4- to 7-membered cycloalkyl, a nitrogen-containing 4- to 7-membered heterocycloalkyl, or alternatively A′ can be directly fused to the ring to which it is attached through R′¹; preferably, A′ is a nitrogen-containing 4- to 7-membered heterocycloalkyl, or alternatively A′ can be directly fused to the ring to which it is attached through R′¹;

L is C₁₋₃alkyl;

R′¹ is hydrogen, C₁₋₃alkyl, or C₃₋₄cycloalkyl;

each R′² is independently C₁₋₃alkyl, fluorine, hydroxyl, C₁₋₃alkoxy, or cyano;

R′³ is hydrogen, C₁₋₃alkyl, or C₃₋₄cycloalkyl;

R′⁴ is a 5- to 10-membered heteroaryl with 1 to 3 heteroatoms, optionally substituted with 1 to 3 R⁴ substituents;

R′⁵ is hydrogen or —N(R⁸)₂;

Z is N or —CR⁹;

each R⁸ is independently hydrogen or C₁₋₃alkyl;

R⁹ is hydrogen, C₁₋₃alkyl, or halogen;

m is 0, 1 or 2;

q is 1, 2, or 3;
and wherein

R″² represents an aryl group optionally substituted with one or more substituents selected from halogen, C₁₋₆alkyl, C₁₋₆alkyloxy, C₁₋₆alkylthio, C₁₋₆fluoroalkyl, C₁₋₆fluoroalkyloxy and —CN;

R″³ represents H, C₁₋₃alkyl, —NR″⁴R″⁵, hydroxyl, or C₁₋₄alkyloxy; or R″³ together with the carbon to which it is attached is N; preferably R″³ together with the carbon to which it is attached is N;

A″ represents C₁₋₇-alkylene optionally substituted with one or two R^{a};

B represents C₁₋₇-alkylene optionally substituted with R^{b};

L″ represents either N substituted with R^{c} or R^{d}, or C substituted with R^{e1} and R^{d} or with two groups R^{e2};

the carbon atoms of A″ and B being optionally substituted with one or more groups R^{t}, which may be identical to or different than each other;

R^{a}, R^{b} and R^{c} are defined such that:
two groups Ra may together form C₁₋₆alkylene;
R^{a} and R^{b} may together form a bond or C₁₋₆alkylene;
R^{a} and R^{c} may together form a bond or C₁₋₆alkylene;
R^{b} and R^{c} may together form a bond or C₁₋₆alkylene;

R^{d} represents a group selected from H, C₁₋₆alkyl, C₃₋₇cycloalkyl, C₃₋₇cycloalkyl-C₁₋₆alkyl, C₁₋₆alkylthio-C₁₋₆alkyl, C₁₋₆alkyloxy-C₁₋₆fluoroalkyl, benzyl, C₁₋₆acyl, and hydroxy-C₁₋₆alkyl;

R^{e1} represents —NR″⁴R″⁵ or a cyclic monoamine optionally comprising an oxygen atom, the cyclic monoamine being optionally substituted with one or more substituents selected from F, C₁₋₆alkyl, C₁₋₆alkyloxy, and hydroxyl;

two groups R^{e2} form, with the carbon atom that bears them, a cyclic monoamine optionally comprising an oxygen atom, this cyclic monoamine being optionally substituted with one or more R^{f}, which may be identical to or different than each other;

R^{f} represents C₁₋₆alkyl, C₃₋₇cycloalkyl, C₃₋₇cycloalkyl C₁₋₃alkyl, hydroxy-C₁₋₆alkyl, C₁₋₆fluoroalkyl or benzyl;

R″⁴ and R″⁵ each independently represent H, C₁₋₄alkyl, C₃₋₇cycloalkyl, or C₃₋₇cycloalkyl-C₁₋₆alkyl;
and wherein

X¹ is selected from O and NQ⁶; provided when X¹ is NQ⁶, Q⁵ and Q⁶ together with the nitrogen atom and the adjacent carbon atom to which they are respectively attached form a heterocyclic ring comprising carbon atoms and zero to 3 additional heteroatoms selected from N, NO⁸, O, S and substituted with 1-5 Q¹⁰;

Q¹ is C₁₋₄alkyl optionally substituted with halogen, OH, CN, and NQ^{a}Q^{a}, or Q¹ is —(CQ^{d}Q^{d})ᵣ-carbocyclyl substituted with 0-5 Q¹¹, and —(CQ^{d}Q^{d})ᵣ-heterocyclyl comprising carbon atoms and 1 to 4 heteroatoms selected from N, NQ⁹, O, S, and substituted with 0-5 Q¹¹;

Q² is selected from H, C₁₋₄alkyl, halogen, CN, aryl, and heteroaryl;

Q³ is selected from H and C₁₋₄alkyl;

Q⁴ is selected from H, C₁₋₄alkyl halogen, and CN;

Q⁵ is selected from H, C₁₋₄alkyl substituted with 0-4 Q^{e}, —(CH₂)ᵣ—C₃₋₆carbocyclyl substituted with 0-4 Q^{e}, and —(CH₂)ᵣ-heterocyclyl comprising carbon atoms and 1 to 3 heteroatoms selected from N, O, S, and substituted with 0-4 Q^{e};

Q⁷ is aryl substituted with 0-3 Q^{e};

Q⁸ is selected from H, C₁₋₄alkyl substituted with 0-3 Q^{e}, —(CH₂)ᵣCN, —(CH₂)ᵣOQ^{b}, —(CH₂)ᵣS(O)ₚQ^{c}, —(CH₂)ᵣC(═O)Q^{b}, —(CH₂)ᵣNQ^{a}Q^{a}, —(CH₂)ᵣC(═O)NQ^{a}Q^{a}, —(CH₂)ᵣC(═O)—C_{1- 4}alkyl substituted with 0-3 Q^{e}, —(CH₂)ᵣNQ^{a}C(═O)Q^{b}, —(CH₂)ᵣNQ^{a}C(═O)OQ^{b}, —(CH₂)ᵣOC(═O)NQ^{a}Q^{a}, —(CH₂)ᵣNQ^{a}C(═O)NQ^{a}Q^{a}, —(CH₂)ᵣC(═O)OQ^{b}, —(CH₂)rS(O)₂NQ^{a}Q^{a} —(CH₂)ᵣNQ^{a}S(O)₂NQ^{a}Q^{a}, —(CH₂)ᵣNQ^{a}S(O)₂Q^{c}, —(CH₂)ᵣ-carbocyclyl substituted with 0-3 Q^{e}, and —(CH₂)ᵣ-heterocyclyl substituted with 0-3 Q^{e};

Q⁹ is selected from H, —C(═O)Q^{b}, C₁₋₆alkyl substituted with 0-5 Q^{e}, —(CH₂)ᵣ—C₃₋₆carbocyclyl substituted with 0-5 Qe, and —(CH₂)ᵣ-heterocyclyl substituted with 0-5 Q^{e};

Q¹⁰ is selected from H, C₁₋₆alkyl substituted with 0-3 Q^{e}, —(CH₂)ᵣNQ^{a}Q^{a} —(CH₂)ᵣC(═O)Q^{b}, —(CH₂)ᵣC(═O)OQ^{b}, —(CH₂)ᵣC(═O)NQ^{a}Q^{a}, —S(O)ₚQ^{c}, —(CH₂)C₃₋₆carbocyclyl substituted with 0-3 Q^{e}, and —(CH₂)^{r}-heterocyclyl substituted with 0-3 Q^{e};

each Q¹¹ is independently selected from H, halogen, ═O, CN, NO₂, —OQ^{b}, —S(O)^{p}Q^{c}, —C(═O)Q_{b}, —(CQ^{d}Q^{d})ᵣNQ^{a}Q^{a}, —(CQ^{d}Q^{d})ᵣC(═O)NQ^{a}Q^{a}, —NQ^{a}C(═O)Q^{b}, —NQ^{a}C(═O)OQ^{b}, —OC(═O)NQ^{a}Q^{a}, —NQ^{a}C(═O)NQ^{a}Q^{a}, —(CQ^{d}Q^{d})^{r}C(═O)OQ^{b}, —S(O)₂NQ^{a}Q^{a}, —NQ^{a}S(O)₂NQ^{a}Q^{a}, —NQ^{a}S(O)₂Q^{c}, C₁₋₆alkyl substituted with 0-5 Q^{e}, —(CQ^{d}Q^{d})ᵣ-C₃₋₆carbocyclyl substituted with 0-5 Q^{e}, and —(CQ^{d}Q^{d})ᵣ-heterocyclyl substituted with 0-5 Q^{e};

each Q^{a} is independently selected from H, CN, C₁₋₆alkyl substituted with 0-5 Q^{e}, C₂₋₆alkenyl substituted with 0-5 Q^{e}, C2-6alkynyl substituted with 0-5 Q^{e}, —(CH₂)ᵣ-C₃₋₁₀carbocyclyl substituted with 0-5 Q^{e}, and —(CH₂)ᵣ-heterocyclyl substituted with 0-5 Q^{e}; or two instances of Q^{a} together with the nitrogen atom to which they are both attached form a heterocyclic ring substituted with 0-5 Q^{e};

each Q^{b} is independently selected from H, C₁₋₆alkyl substituted with 0-5 Q^{e}, C₂₋₆alkenyl substituted with 0-5 Q^{e}, C₂₋₆alkynyl substituted with 0-5 Q^{e}, —(CH₂)ᵣ—C₃₋₁₀carbocyclyl substituted with 0-5 Q^{e}, and —(CH₂)ᵣ-heterocyclyl substituted with 0-5 Q^{e};

each Q^{c} is independently selected from C₁₋₆alkyl substituted with 0-5 Q^{e}, C₂₋₆alkenyl substituted with 0-5 Q^{e}, C₂₋₆alkynyl substituted with 0-5 Q^{e}, C₃₋₆carbocyclyl substituted with 0-5 Q^{e}, and heterocyclyl substituted with 0-5 Q^{e};

each Q^{d} is independently selected from H and C₁₋₄alkyl substituted with 0-5 Q^{e};

each Q^{e} is independently selected from C₁₋₆alkyl substituted with 0-5 Q^{f}, C₂₋₆alkenyl, C₂₋₆alkynyl, —(CH₂)ᵣ—C₃₋₆cycloalkyl, halogen, CN, NO₂, ═O, CO₂H, —(CH₂)ᵣOQ^{f}, SQ^{f}, and —(CH₂)ᵣNQ^{f}Q^{f};

each O^{f} is independently selected from H, F, C₁₋₅alkyl, C₃₋₆cycloalkyl, and phenyl, or two instances of O^{f} together with the nitrogen atom to which they are both attached form a heterocyclic ring optionally substituted with C₁₋₄alkyl;

each p is independently 0, 1, or 2; and

each r is independently 0, 1, 2, 3, or 4,
and wherein

X² is selected from —NH—, —CH₂—, —CH(Ph)—, —CH₂CH₂—, —CH₂CH(Ph)—, —CH═CH—, —CH₂OCH₂—, —CH₂NHC(O)—, —CH₂NHC(O)CH(Ph)— and —CH₂NHC(O)CH₂—,

Q′¹ is selected from Q′⁶, halogen, —CF₃, —OCF₃, —OQ′⁶, —CO₂Q′⁶, —SO₂N(Q′⁶)₂, and —NO₂;

Q′², Q′³, Q′⁴, and Q′⁵ are independently selected from H, halogen, C₁₋₆alkoxy, —NH₂, —NHQ′6, —CN, —NO₂, —OCF₃, and —CO₂Q′⁶; wherein

Q′⁶ is selected from H and C₁₋₆alkyl; and wherein when X² is —CH(Ph)—, —CH₂CH(Ph)— or —CH₂NHC(O)CH(Ph)—, then Q′², Q′³, Q′⁴, and Q′⁵ are H,

e¹ is S, O, or NR⁵, preferably S, O, or NH;

e² and e′² are each independently H, halogen, C₁₋₃alkyl, halogenated C₁₋₃alkyl, C₁₋₃alkoxy, S(═O)₀₋₂C₁₋₃alkyl, and [C(═O)O]C₁₋₃alkyl, or e² and e′² together form a 3-6-membered cyclic structure; preferably at least one of e² and e′² is not H; more preferably e² and e′² are each independently H, trifluoromethyl, S(O)2CH3, C(═O)OCH3, CI, F, or together form —O—CF₂—O—;

e³ is substituted phenyl, substituted pyridinyl, substituted thiazolyl, substituted oxazolyl, or —CH₂—S—2-((3-(e⁴)-4-oxo-3,4,6,7-tetrahydrothieno[3,2-d]pyrimidin-2-yl); wherein substitutions in e³ are preferably —OR⁵, halogenated —OR⁵, —NH₂, —NHR⁵, N(CH₃)R⁵, tetrazolyl, C(═O)OR⁵, or —NH—C(═O)-e⁵; wherein a —NH—C(═O)-e⁵ moiety is preferably at the 2-position of a singly substituted thiazolyl, more preferably said substituted thiazolyl is thiazol-4-yl;

e⁴ is phenyl, substituted phenyl, benzyl, or substituted benzyl, wherein a substitution in e⁴ is preferably C₁₋₃alkyl, C₁₋₃alkoxy, halogenated C₁₋₃alkyl, or halogenated C₁₋₃alkoxy, more preferably methoxy, trifluoromehtyl, or trifluoromethoxy; most preferably e⁴ is phenyl, benzyl, p-methoxybenzyl, o,m-dimethoxybenzyl, m-trifluoromethylbenzyl, p-trifluoromethylbenzyl, or p-trifluoromethoxybenzyl;

e⁵ is phenyl, substituted phenyl, pyrimidyl, or substituted pyrimidyl, furyl, or substituted furyl, wherein a substitution in e⁵ is preferably C₁₋₃alkyl, C₁₋₃alkoxy, halogenated C₁₋₃alkyl, or halogenated C₁₋₃alkoxy, more preferably —OCF₃; preferably e⁵ is 2-trifluoromethoxyphenyl, pyridine-4-yl, or furan-2-yl, most preferably 2-trifluoromethoxyphenyl;

q¹ is H or a C₂₋₈hydrocarbon optionally substituted with 1-5 halogens, preferably a C₂₋₈ hydrocarbon optionally substituted with 1-5 halogens, wherein halogens are preferably fluor, wherein the C₂₋₈ hydrocarbon is preferably ethyl, isopropyl, phenyl, thiophenyl, pyridinyl, or toluyl, wherein toluyl is preferably o-toluyl, wherein toluyl is preferably halogenated, more preferably trihalogenated at its methyl moiety, wherein phenyl is preferably halogenated, more preferably it is m-fluorophenyl, p-fluorophenyl, m,p-difluorophenyl, o,m-difluorophenyl, m,m-difluorophenyl, o,p-difluorophenyl, or 2,5-difluorophenyl, wherein thiophenyl is preferably 3-thiophenyl, wherein pyridinyl is preferably halogenated or methylated, wherein halogenated pyridinyl is preferably 2-fluoropyridinyl such as 2-fluoropyridin-4-yl or 6-chloropyridinyl such as 6-chloropyridin-3-yl; wherein methylated pyridinyl is preferably 2-methylpyridinyl such as 2-methylpyridin-4-yl; most preferably q¹ is isopropyl, m-fluorophenyl, or pyridine-3-yl;

q² is H, NH₂, NHq³, or Nq³q⁴; more preferably q² is H, N-morpholinyl, 4-methyl-1-piperazinyl, or 1-piperazinyl, most preferably H or N-morpholinyl;

q³ is —(CH₂)ᵣ—OH, —(CH₂)ᵣ—H, or —(CH₂)ᵣ—Nq³′q⁴′; preferably q³ is —CH₂CH₂OH, —CH₂CH₂CH₂CH₃, —CH₂CH₂CH₂Nq^{3′}q^{4′}, —CH₂CH₂Nq^{3′}q^{4′}, or —CH2Nq^{3′}q^{4′}; wherein q^{3′} and q⁴′ together form a 5-7 membered cyclic hydrocarbon, preferably a 6-membered cyclic hydrocarbon, wherein the cyclic hydrocarbon is optionally substituted with one or more methoxy, hydroxymethyl, amino, methylamino, dimethylamino, or oxo moieties, wherein the cyclic hydrocarbon can comprise heteroatoms in its ring in addition to the nitrogen of q², preferably O or S; preferably when q⁴ and q³ together form a cyclic hydrocarbon, q⁴ and q³ represent —CH₂CH₂OCH₂CH₂—, —CH₂CH₂SCH₂CH₂—, —CH₂CH₂(SO₂)CH₂CH₂—, —CH₂CH₂(SO)CH₂CH₂—, —CH₂CH₂(CO)CH₂CH₂—, —CH₂CH₂(C[NHCH₃])CH₂CH₂—, —CH₂CH₂(C[NH_{2]})CH₂CH₂—, —CH₂CH₂(C[N(CH₃)_{2]})CH₂CH₂—, —CH₂CH₂NHCH₂CH₂—, —CH₂CH₂(NCH₃)CH₂CH₂—, —CH₂CH₂(C[CH₂OH])CH₂CH₂—, or —CH₂CH₂(COCH₃)CH₂CH₂—;

or when q⁴ is present, q³ together with q⁴ form a 5-7 membered cyclic hydrocarbon, preferably a 6-membered cyclic hydrocarbon, wherein the cyclic hydrocarbon is optionally substituted with one or more methoxy, hydroxymethyl, amino, methylamino, dimethylamino, or oxo moieties, wherein the cyclic hydrocarbon can comprise heteroatoms in its ring in addition to the nitrogen of q², preferably O or S; preferably when q⁴ and q³ together form a cyclic hydrocarbon, q⁴ and q³ represent —CH₂CH₂OCH₂CH₂—, —CH₂CH₂SCH₂CH₂—, —CH₂CH₂(SO₂)CH₂CH₂—, —CH₂CH₂(SO)CH₂CH₂—, —CH₂CH₂(CO)CH₂CH₂—, —CH₂CH₂(C[NHCH₃])CH₂CH₂—, —CH₂CH₂(C[NH₂])CH₂CH₂—, —CH₂CH₂(C[N(CH₃)₂])CH₂CH₂—, —CH₂CH₂NHCH₂CH₂—, —CH₂CH₂(NCH₃)CH₂CH₂—, CH₂CH₂(C[CH₂OH])CH₂CH₂—, or —CH₂CH₂(COCH₃)CH₂CH₂—;

c¹ and c² together with the carbon atom to which they are attached form an optionally substituted 5-10-membered cyclic structure, wherein the optional substitution is preferably methyl, trifluoromethyl, halogen, phenyl, 1-piperazinyl, 4-methyl-1-piperazinyl, or methoxy, more preferably trifluoromethyl, halogen, or methoxy, wherein the 5-10-membered cyclic structure is preferably phenyl, pyridinyl such as pyridine-2-yl or pyridine-3-yl, indolyl such as indol-2-yl, benzimidazolyl such as benzimidazol-2-yl, benzothiazolyl such as benzothiazol-2-yl, benzoxazolyl such as benzoxazol-2-yl, indenyl such as inden-2-yl, imidazolyl such as imidazole-2-yl, thiazolyl such as thiazol-2-yl, or oxazolyl such as oxazol-2-yl; more preferably the 5-10-membered cyclic structure is phenyl, trifluoromethylphen-3-yl, fluorophen-2-yl, benzimidazol-2-yl, 5-chlorobenzimidazol-2-yl, 5,6-dichlorobenzimidazol-2-yl, 5-fluorobenzimidazol-2-yl, 5,6-difluorobenzimidazol-2-yl, 5-methoxybenzimidazol-2-yl, 5,6-dimethoxybenzimidazol-2-yl, pyridin-2-yl, pyridin-3-yl, 6-methylpyridin-2-yl, 5-methylpyridin-2-yl, imidazol-2-yl, oxazol-2-yl, thiazol-2-yl, 4-phenylimidazol-2-yl, 4-phenyloxazol-2-yl, 4-phenylthiazol-2-yl, 4,5-difluorobenzimidazol-2-yl, 4,5-dichlorobenzimidazol-2-yl, 4,5-dimethoxybenzimidazol-2-yl, 1-(1-methylpiperazin-4-yl)phen-4-yl, 3-(1-methylpiperazin-4-yl)pyridin-6-yl, benzothiazol-2-yl, or benzoxazol-2-yl; most preferably the 5-10-membered cyclic structure is phenyl, m-trifluoromethylphenyl, or 4,5-difluorobenzimidazol-2-yl;

or isomers or pharmaceutically acceptable salts thereof.

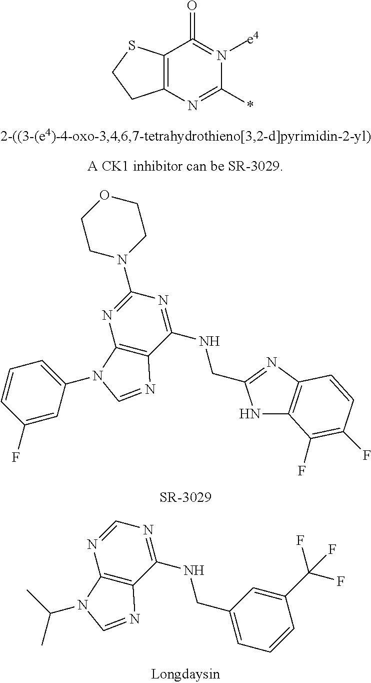

In preferred embodiments, the CK1 inhibitor is of general formula (Ia) or (Ib), or isomers or pharmaceutically acceptable salts thereof, wherein X, Y, A, R¹, R², R³, R⁴, R⁵, R⁶, R⁷, n, t, u, A′, L, R′¹, R′², R′³, R′⁴, R′⁵, Z, R⁸, R⁹ , m, and q and other variables are as defined above. In a further preferred embodiment, it is of general formula (Ia), or isomers or pharmaceutically acceptable salts thereof, wherein X, Y, A, R¹, R², R³, R⁴, R⁵, R⁶, R⁷, n, t, u, A′, L, R′¹, R′², R′³, R′⁴, R′⁵, Z, R⁸, R⁹ , m, and q and other variables are as defined above. In a further preferred embodiment, it is of general formula (Ib), or isomers or pharmaceutically acceptable salts thereof, wherein X, Y, A, R¹, R², R³, R⁴, R⁵, R⁶, R⁷, n, t, u, A′, L, R′¹, R′², R′³, R′⁴, R′⁵, Z, R⁸, R⁹ , m, and q and other variables are as defined above. CK1 inhibitors of this class are known per se in the art and have their structure and synthesis described in more detail in, for example, WO2011051858, WO2012085721, and WO2015119579, or in Halekotte et al., Molecules 2017, DOI: 10.3390/molecules22040522 , or in Luxenburger et al., Molecules 2019, DOI: 10.3390/molecules24050873, or in Peifer et al., J. Med. Chem. 2009, 52, 7618-7630 DOI: 10.1021/jm9005127.

Preferably, when R⁷ is —Nr⁷r′⁷, Y is NH, X is C(S—CH₃) or C([S═O]—CH₃), R⁴ is F and para, n is 1, A is absent, and one of r⁷ and r′⁷ is H. Also, preferably, when R⁷ is —Nr⁷r′⁷, X is —O—, Y is C(isopropyl), R⁴ is F and para, n is 1, A is absent, and one of r⁷ and r′⁷ is H.

CK1 inhibitors of this class comprise an azole core. In preferred embodiments of this aspect, the casein kinase 1 inhibitor is from the class comprising an azole core. More preferably, these CK1 inhibitors for use comprise a 4-aryl-5-heteroaryl-1-heterocycloalkyl-imidazole moiety. Preferably, for these inhibitors, a single R⁴ is present, para to the azole core; more preferably this R⁴ is F. Accordingly, in further more preferred embodiments, the casein kinase 1 inhibitor comprises an azole core linked to a 4-halophenyl moiety, preferably a 4-fluorophenyl moiety. Highly preferred compounds comprising an azole core are compounds D, E, F, and G as shown in table 3; compound D is even more preferred. Other preferred compounds in this class are the compounds shown in table 1 of Halekotte et al., the compounds shown in table 1 of Luxenburger et al., the compounds shown in table 3 of Luxenburger et al, the compounds shown in table 3 of Peifer et al., and the compounds in table 4 of Peifer et al.

In preferred embodiments, the CK1 inhibitor is of general formula (2a) or (2b), or isomers or pharmaceutically acceptable salts thereof, wherein R⁵, R⁶, R″², R″³, A″, B, L″, R^{a}, R^{b}, R^{c}, R^{d}, R^{e1}, R^{e2}, R^{f}, R″⁴, R″⁵, X¹, Q¹, Q², Q³, Q⁴, Q⁵, Q⁶, Q⁷, Q⁸, Q⁹, Q¹⁰, Q¹¹, Q^{a}, Q^{b}, Q^{c}, Q^{d}, Q^{e}, Q^{t}, r, and p are as defined above. In a further preferred embodiment, it is of general formula (2a) or isomers or pharmaceutically acceptable salts thereof, wherein R⁵, R⁶, R″², R″³, A″, B, L″, R^{a}, R^{b}, R^{c}, R^{d}, R^{e1}, R^{e2}, R^{f}, R″⁴, R″⁵, X¹, Q¹, Q², Q³, Q⁴, Q⁵, Q⁶, Q⁷, Q⁸, Q⁹, Q¹⁰, Q¹¹, Q^{a}, Q^{b}, Q^{c}, Q^{d}, Q^{e}, Q^{f}, p, and r are as defined above. In a further preferred embodiment, it is of general formula (2b) or isomers or pharmaceutically acceptable salts thereof, wherein R⁵, R⁶, R″², R″³, A″, B, L″, R^{a}, R^{b}, R^{c}, R^{d}, R^{e1}, R^{e2}, R^{f}, R″⁴, R″⁵, X¹, Q¹, Q², Q³, Q⁴, Q⁵, Q⁶, Q⁷, Q⁸, Q⁹, Q¹⁰, Q¹¹, Q^{a}, Q^{b}, Q^{c}, Q^{d}, Q^{e}, Q^{f}, p, and r are as defined above. CK1 inhibitors of this class are known per se in the art and have their structure and synthesis described in more detail in, for example, WO2009016286 and WO2015195880 and WO2009037394 and WO2010/130934.

CK1 inhibitors of this class comprise an imidazo[1,2-b]pyridazine core. In preferred embodiments of this aspect, the casein kinase 1 inhibitor is from the class comprising a imidazo[1,2-b]pyridazine core. CK1 inhibitors with an imidazo[1,2-b]pyridazine core more preferably have a 3-(pyrid-4-yl)imidazo[1,2-b]pyridazine core, even more preferably a 6-cyclo-3-(pyrid-4-yl)imidazo[1,2-b]pyridazine core. In further preferred embodiments, the casein kinase 1 inhibitor comprises an azole core or comprises an imidazo[1,2-b]pyridazine core. CK1 inhibitors of general formula (2a) wherein R″³ together with the carbon to which it is attached forms N are known from are known from WO2009037394. A preferred such compound wherein these atoms form N is the following:

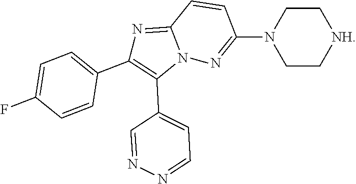

The following compound:

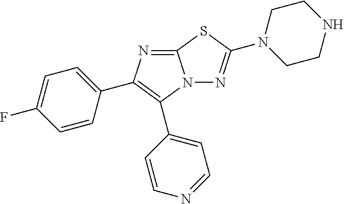 [SMILES: Fc1ccc(-c2nc3sc(N4CCNCC4)nn3c2-c2ccncc2)cc1]

is a preferred compound wherein the compound is of general formula (2a) and R⁵ and R⁶ together with the carbon atoms to which they are attached form —S—; such compounds are known from WO2010/130934.

In further preferred embodiments, the casein kinase 1 inhibitor is of general formula (1 a), (1b), (2a), or (2b), wherein X, Y, A, R¹, R², R³, R⁴, R⁵, R⁶, R⁷, n, t, u, A′, L, R′¹, R′², R′³, R′⁴, R′⁵, Z, R⁸, R⁹ ,m, q, R″², R″³, A″, B, L″, R^{a}, R^{b}, R^{c}, R^{d}, R^{e1}, R^{e2}, R^{t}, R″⁴, R″⁵, X¹, Q¹, Q², Q³, Q⁴, Q⁵, Q⁶, Q⁷, Q⁸, Q⁹, Q¹⁰, Q¹¹, Q^{a}, Q^{b}, Q^{c}, Q^{d}, Q^{e}, Q^{f}, r, and p are as defined above. In further preferred embodiments, the casein kinase 1 inhibitor is of general formula (1a), (1 b), (2a), (2b), (3a), or (3b) wherein variables are as defined above. In further preferred embodiments, the casein kinase 1 inhibitor is of general formula (1a), (1b), (2a), (2b), (3a), (3b), or (4) wherein variables are as defined above. In further preferred embodiments, the casein kinase 1 inhibitor is of general formula (1 a), (1b), (3a), or (3b) wherein variables are as defined above. In further preferred embodiments, the casein kinase 1 inhibitor is of general formula (1a), (1b), (3a), (3b), or (4) wherein variables are as defined above. In further preferred embodiments, the casein kinase 1 inhibitor is of general formula (2a), (2b), (3a), or (3b) wherein variables are as defined above. In further preferred embodiments, the casein kinase 1 inhibitor is of general formula (2a), (2b), (3a), (3b), or (4) wherein variables are as defined above. In further preferred embodiments, the casein kinase 1 inhibitor is of general formula (1a), (1b), or (4) wherein variables are as defined above. In further preferred embodiments, the casein kinase 1 inhibitor is of general formula (2a), (2b), or (4) wherein variables are as defined above. In further preferred embodiments, the casein kinase 1 inhibitor is of general formula (3a), (3b), or (4) wherein variables are as defined above. In further preferred embodiments, the casein kinase 1 inhibitor is of general formula (1a), (1 b), (2a), (2b), or (4) wherein variables are as defined above.

In preferred embodiments, the CK1 inhibitor is of general formula (3a) or (3b) or isomers or pharmaceutically acceptable salts thereof, wherein X², Q′¹, Q′², Q′³, Q′⁴, Q′⁵, and Q′⁶ and other variables are as defined above. CK1 inhibitors of this class are known in the art per se and have their structure and synthesis described in more detail in, for example, EP2949651, Bischof et al., Amino Acids (2012) 43:1577-1591 DOI: 10.1007/s00726-012-1234-x, Garcia-Reyes et al., J. Med. Chem. 2018, 61, 4087-4102, DOI: 10.102¹/ₐcs.jmedchem.8b00095, and Richter et al., J. Med. Chem., DOI: 10.1021/jm500600b. When a CK1 inhibitor is of general formula (3a), X² is preferably —CH₂—, —CH₂CH₂—, —CH(Ph)—, or —NH—, most preferably —CH₂—; Q′1 is preferably —CF₃, halogen, or C₁₋₆alkyl, more preferably —CF₃; Q′₂, Q′₃, Q′₄, and Q′⁵ are preferably independently selected from H, halogen, and C₁₋₅alkoxy. More preferably, when a CK1 inhibitor is of general formula (3a), X² is —CH₂— and Q′¹ is —CF₃.

In further preferred embodiments, the CK1 inhibitor is of general formula (3a). In further preferred embodiments, the CK1 inhibitor is of general formula (3b). Preferred CK1 inhibitors of general formula (3b) are compounds 17-23 in Scheme 1 of Garcia-Reyes et al., the compounds in table 1 of Bischof et al., and the compounds in table 1 of Richter et al. Specifically preferred CK1 inhibitors of general formula (3b) are compounds 17-23 in Scheme 1 of Garcia-Reyes et al. Specifically preferred CK1 inhibitors of general formula (3b) are the compounds in table 1 of Bischof et al. Specifically preferred CK1 inhibitors of general formula (3b) are the compounds in table 1 of Richter et al.

CK1 inhibitors of this class comprise a 3-heteroindole core. In preferred embodiments, the casein kinase 1 inhibitor is from the class comprising a 3-heteroindole core core. CK1 inhibitors with a 3-heteroindole core more preferably have a benzimidazole core or a benzothiazole core or a benzoxazole core. In further preferred embodiments, the casein kinase 1 inhibitor comprises an azole core or comprises an imidazo[1,2-b]pyridazine core or comprises a 3-heteroindole core.

In preferred embodiments, the CK1 inhibitor is of general formula (4) or isomers or pharmaceutically acceptable salts thereof, wherein q¹, q¹, q³, q⁴, q^{3′}, q^{4′}, c¹, and c² are as defined above. CK1 inhibitors of this class are known in the art per se and have their structure and synthesis described in more detail in, for example, Hirota et al., PLoS Biol 8(12): e1000559. doi:10.1371/journal.pbio.1000559—and in Monastyrskyi et al., Bioorg. Med. Chem. 2018 590-602 https://doi.org/10.1016/j.bmc.2017.12.020. When a CK1 inhibitor is of general formula (4), it is preferably SR-3029 or longdaysin, more preferably longdaysin. In other preferred embodiments a CK1 inhibitor of general formula (4) is not SR-3029.

CK1 inhibitors of this class comprise a 6-aminopurine core. In preferred embodiments of this aspect, the casein kinase 1 inhibitor is from the class comprising a 6-aminopurine core. In further preferred embodiments, the casein kinase 1 inhibitor comprises an azole core or comprises an imidazo[1,2-b]pyridazine core or comprises a 6-aminopurine core. In further preferred embodiments, the casein kinase 1 inhibitor comprises an azole core or comprises a 6-aminopurine core. In further preferred embodiments, the casein kinase 1 inhibitor comprises an imidazo[1,2-b]pyridazine core or comprises a 6-aminopurine core. In further preferred embodiments, the casein kinase 1 inhibitor comprises an azole core or comprises an imidazo[1,2-b]pyridazine core or comprises a 6-aminopurine core or comprises a 3-heteroindole core.

In preferred embodiments the CK1 inhibitor is of general formula (1a), (2a), (3b), or (4), wherein
X is CR¹ and the R¹ in that CR¹ moiety it is —S—(CH₂)₀₋₃CH₃ or —(S═O)—(CH₂)₀₋₃CH₃, or wherein
R″³ together with the carbon to which it is attached is N, or wherein
when the compound is of general formula (2a), R⁵ and R⁶ can, together with the carbon atoms to which they are attached, form —S— or —O—, preferably —S—, or wherein
R⁷ is —Nr⁷r^{′7}, or wherein
X or Y is —O—,
and wherein other variables are as described above.

Structures of exemplary CK1 inhibitors are shown in table 3. In further preferred embodiments, the casein kinase 1 inhibitor is selected from the group consisting of compounds A, B, C, D, E, F, G, H, I, J, K, L, M, N, O, SR-3029, PF-670462, and PF-5006739. Compound O is also known as TA-01. More preferably, the casein kinase 1 inhibitor is selected from the group consisting of compounds A, B, C, D, E, F, G, H, O, SR-3029, PF-670462, and PF-5006739. Even more preferably, the casein kinase 1 inhibitor is selected from the group consisting of compounds A, D, F, G, H, O, SR-3029, PF-670462, and PF-5006739. Even more preferably, the casein kinase 1 inhibitor is selected from the group consisting of compounds A, D, F, G, H, SR-3029, PF-670462, and PF-5006739. Most preferably, the casein kinase 1 inhibitor is selected from the group consisting of compounds A, D, F, G, H, SR-3029, and PF-5006739. It is also highly preferred that the casein kinase 1 inhibitor be compound D. It is also highly preferred that the casein kinase 1 inhibitor is selected from the group consisting of compounds A, B, and H, more preferably it is compound H.

In other embodiments, the CK1 inhibitor is an inhibitory antibody, an antisense oligonucleotide, or an oligonucleotide that prevents expression of CK1.

The various isoforms of casein kinase 1 are known to have different functions. Within the set of known isoforms, CK1δ and CK1ε are preferred targets for the CK1 inhibitors according to the invention. These two isoforms are known to be closely related to one another. For example, CK1δ and CK1ε were thought to be generally redundant in circadian cycle length and protein stability, but were later revealed to have slightly different functions (Etchegaray J P et al., 2009, DOI:10.1128/MCB.00338-09). Due to their physiological importance, and the known efficacy of the CK1 inhibitors, in preferred embodiments the casein kinase inhibitor inhibits at least casein kinase 16 or casein kinase 1 e. Optionally, the casein kinase inhibitor is specific for casein kinase 16 or for casein kinase 1ε. Furthermore, in more preferred embodiments the casein kinase inhibitor at least inhibits, and optionally is specific for, casein kinase 1δ. In other more preferred embodiments the casein kinase inhibitor at least inhibits, and optionally is specific for, casein kinase 1ε. In other embodiments the casein kinase inhibitor at least inhibits, and optionally is specific for, casein kinase 1α. In other embodiments the casein kinase inhibitor at least inhibits, and optionally is specific for, casein kinase 1β. In other embodiments the casein kinase inhibitor at least inhibits, and optionally is specific for, casein kinase 1γ1, 1γ2, and/or 1γ3. It is to be understood in this context that a CK1 inhibitor is specific for a particular isoform when it at least partially inhibits that particular isoform. Preferably, it inhibits that particular isoform more efficiently than other isoforms.

CK1 inhibitors suitable for use in the invention preferably have an IC₅₀ on a casein kinase of at most 650 nM, preferably of at most 500 nM, more preferably of at most 400 nM, even more preferably of at most 300 nM, still more preferably of at most 250 nM, still more preferably of at most 200 nM, most preferably of at most 100 nM. In preferred embodiments, the CK1 inhibitor has an IC₅₀ on at least casein kinase 1δ or casein kinase 1ε of at most 450 nM, more preferably of at most 400 nM, even more preferably of at most 350 nM, more preferably still of at most 200 nM, even more preferably still of at most 100 nM, most preferably of at most 50 nM. In most preferred embodiments the CK1 inhibitor has an IC₅₀ on casein kinase 1δ of at most 350 nM, preferably at most 100 nM, more preferably at most 35 nM, most preferably at most 25 nM. IC₅₀ values for CK1 can be determined using any method known in the art, for example as described in WO2011051858, WO2015119579, EP2949651, or US2005/0131012. Suitable assays can use a peptide substrate and a readout method, for example using the Kinase-Glo assay (Promega, part #V672A).

### p38 Inhibitors

Inhibitors of p38 mitogen-activated protein kinases, referred to herein as p38 inhibitors, are generally known in the art. Preferred p38 are small molecules. Within the context of the invention, examples of preferred p38 inhibitors are those mentioned herein.

In preferred embodiments, the p38 inhibitor inhibits p38-α. In preferred embodiments, the p38 inhibitor inhibits p38-β. In more preferred embodiments, the p38 inhibitor inhibits p38-α and p38-β. In particularly preferred embodiments, the p38 inhibitor does not inhibit p38-γ, or does not significantly inhibit p38-γ, or inhibits p38-γat most 50%, preferably at most 20%, most preferably at most 10%.

A variety of p38 inhibitors are known and available, and some are in clinical development. Any of these may be used. In preferred embodiments, a p38 inhibitor is selected from the group consisting of ARRY-797, VX-745, VX-702, RO-4402257, SCID-469, BIRB-796, SD-0006, PH-797804, AMG-548, LY2228820, SB-681323 and GW-856553 (losmapimod). In further preferred embodiments p38 inhibitors are selected from the group consisting of N-(4-(2-ethyl-4-(m-tolyl)thiazol-5-yl)pyridin-2-yl)benzamide; 2-(2,4-difluorophenyl)-6-(1-(2,6-difluorophenyl)ureido)nicotinamide; 6-(2,4-difluorophenoxy)-8-methyl-2-((tetrahydro-2H-pyran-4-yl)amino)pyrido[2,3-d]pyrimidin-7(8H)-one; 6-(2,4-difluorophenoxy)-2-((1,5-dihydroxypentan-3-yl)amino)-8-methylpyrido[2,3-d]pyrimidin-7(8H)-one; (R)-6-(2-(4-fluorophenyl)-6-(hydroxymethyl)-4,5,6,7-tetrahydropyrazolo[1,5-a]pyrimidin-3-yl)-2-(o-tolyl)pyridazin-3(2H)-one; 6-(5-(cyclopropylcarbamoyl)-3-fluoro-2-methylphenyl)-N-neopentylnicotinamide; 5-(2-(tert-butyl)-4-(4-fluorophenyl)-1H-imidazol-5-yl)-3-neopentyl-3H-imidazo [4,5-b]pyridin-2-amine; 2-(6-chloro-5-((2R,5S)-4-(4-fluorobenzyl)-2,5-dimethylpiperazine-1-carbonyl)-1-methyl-1H-indol-3-yl)-N,N-dimethyl-2-oxoacetamide; 1-(3-(tert-butyl)-1-(p-tolyl)-1H-pyrazol-5-yl)-3-(4-(2-morpholinoethoxy)naphthalen-1-yl)urea; 4-((5-(cyclopropylcarbamoyI)-2-methylphenyl)amino)-5-methyl-N-propylpyrrolo[2,1-f][1,2,4]triazine-6-carboxamide; 3-(3-bromo-4-((2,4-difluorobenzyl)oxy)-6-methyl-2-oxopyridin-1(2H)-yl)-N,4-dimethylbenzamide; 1-(3-(tert-butyl)-1-(p-tolyl)-1H-pyrazol-5-yl)-3-(5-fluoro-2-((1-(2-hydroxyethyl)-1H-indazol-5-yl)oxy)benzyl)urea; 8-(2,6-difluorophenyl)-2-((1,3-dihydroxypropan-2-yl)amino)-4-(4-fluoro-2-methylphenyl)pyrido[2,3-d]pyrimidin-7(8H)-one; 5-(2,6-dichlorophenyl)-2-((2,4-difluorophenyl)thio)-6H-pyrimido[1,6-b]pyridazin-6-one; (5-(2,4-difluorophenoxy)-1-isobutyl-1H-indazol-6-yl)((2-(dimethylamino)ethyl)-12-azaneyOmethanone; and (R)-2-((2,4-difluorophenyl)amino)-7-(2,3-dihydroxypropoxy)-10,11-dihydro-5H-dibenzo[a,d][7]annulen-5-one.

Examples of suitable p38 inhibitors are ARRY-797 (CHEMBL1088750, CAS: 1036404-17-7), LOSMAPIMOD (CHEMBL1088752, CAS: 585543-15-3), AZD-7624 (CHEMBL9960, CAS: 1095004-78-6), DORAMAPIMOD (CHEMBL103667), NEFLAMAPIMOD (CHEMBL119385, CAS: 209410-46-8), TAK-715 (CHEMBL363648, CAS: 303162-79-0), TALMAPIMOD (CHEMBL514201, CAS: 309913-83-5), PAMAPIMOD (CHEMBL1090089, CAS: 449811-01-2), VX-702 (CHEMBL1090090, CAS: 745833-23-2), PH-797804 (CHEMBL1088751, CAS: 586379-66-0), BMS-582949 (CHEMBL1230065, CAS: 623152-17-0), PF-03715455 (CHEMBL1938400, CAS: 1056164-52-3), DILMAPIMOD (CHEMBL2103838, CAS: 444606-18-2), SEMAPIMOD (CHEMBL2107779, CAS: 352513-83-8), RALIMETINIB (CHEMBL2364626, CAS: 862505-00-8), FX-005 (CHEMBL3545216, CAS: 2016822-86-7), ACUMAPIMOD (CHEMBL3545226, CAS: 836683-15-9), KC-706 (CHEMBL3545282, CAS: 896462-15-0), PG-760564 (CHEMBL3545398), RWJ-67657 (CHEMBL190333, CAS: 215303-72-3), RO-3201195 (CHEMBL203567, CAS: 249937-52-8), AMG-548 (CH EMBL585902, CAS: 864249-60-5), SD-0006 (CHEMBL1090173), SCIO-323 (CHEMBL1614702, CAS: 309913-51-7), R-1487 (CHEMBL1766582, CAS: 449808-64-4), AZD-6703 (CHEMBL2031465, CAS: 1083381-65-0), SC-80036 (CHEMBL3544930), GSK-610677 (CHEMBL3544968, CAS: 2016840-17-6), LY-3007113 (CHEMBL3544998), LEO-15520 (CHEMBL3545074), AVE-9940 (CHEMBL3545117, CAS: 1201685-00-8), PS-516895 (CHEMBL3545139), TA-5493 (CHEMBL3545201, CAS: 1073666-93-9), PEXMETINIB (ARRY614) (CHEMBL3545297, CAS: 945614-12-0), and SB-85635 (CHEMBL3545384).

In preferred embodiments, the p38 inhibitor is selected from one or more of Formulae pI, pII, pIII, pIV, pV, pVI, pVII, pVIII, pIX, pX, pXI, pXII, and pXIII (of the corresponding genuses described below), or a stereoisomer thereof, an isotopically-enriched compound thereof, a prodrug thereof, a solvate thereof, or a pharmaceutically acceptable salt thereof.

Compounds of Genus pI can be prepared according to the disclosure of U.S. Pat. No. 7,276,527. Genus pl is characterized by optionally N-oxidized compounds of Formula (pI) or stereoisomers thereof, isotopically-enriched compounds thereof, prodrugs thereof, solvates thereof, and pharmaceutically acceptable salts thereof.

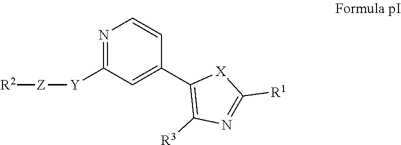

wherein:

R¹ is selected from (i), (ii), (iii), or (iv): (i) hydrogen, (ii) a group selected from C₁₋₆alkyl, C₂₋₆alkenyl, C₂₋₆alkynyl, C₃₋₆cycloalkyl, C₆₋₁₄aryl, and C₇₋₁₆ aralkyl group; wherein the alkyl, alkenyl, alkynyl, cycloalkyl, aryl, or aralkyl is optionally substituted with one or more substituents selected from a Substituent Group A, (iii) —(C═O)—R⁵ —(C═O)-0R⁵—(C═O)—NR⁵R⁶—(C═S)—NHR⁵, or —SO₂—R⁷, wherein:

R⁵ is hydrogen, C₁₋₆alkyl, C₂₋₆alkenyl, C₂₋₆alkynyl, C₃₋₆cycloalkyl, C₆₋₁₄aryl, or _{c7-16} aralkyl. wherein the alkyl, alkenyl, alkynyl, cycloalkyl, aryl, or aralkyl is optionally substituted with one or more substituents selected from the Substituent Group A,

R^{e} is hydrogen or C₁₋₆alkyl.

R7 is C₁₋₆alkyl, C₂₋₆alkenyl, C₂₋₆alkynyl, C₃₋₆cycloalkyl, C₆₋₁₄aryl, or _{c7-16} aralkyl. wherein the alkyl, alkenyl, alkynyl, cycloalkyl, aryl, or aralkyl is optionally substituted with one or more substituents selected from the Substituent Group A

(iv) an amino group optionally substituted with substituents selected from (a), (b), or (c): (a) C₁₋₆alkenyl, C₂₋₆alkynyl, C₂₋₆cycloalkyl, C₆₋₁₄aryl, or _{c7-16} aralkyl. wherein the alkyl, alkenyl, alkynyl, cycloalkyl, aryl, or aralkyl is optionally substituted with one or more substituents selected from the Substituent Group A; (b) —(C═O)—R⁵—(C═O)—0R⁵—(C═O)—NR⁵R⁶—(C═S)—NHR⁵, or —SO₂—R⁷, and (c) C₁₋₆alkylidene optionally substituted with one or more substituents selected from the Substituent Group A;

R² is a C₆₋₁₄ monocyclic or fused polycyclic aryl optionally substituted with one or more substituents selected from the Substituent Group A;

R³ is hydrogen or C6-14aryl, wherein the aryl is optionally substituted with one more substituents selected from the Substituent Group A;

X is —S—, S(O)—, or S(O)₂—;

Y is a bond, —O—, —S—S(O)—, S(O)₂— or NR^{e},

wherein R⁴ is:

(a) hydrogen; (b) C₁₋₆alkyl, C₂₋₆alkenyl, C₂₋₆alkynyl, C₃₋₆cycloalkyl, C₆₋₁₄aryl, or _{c7-16} aralkyl. wherein the alkyl, alkenyl, alkynyl, cycloalkyl, aryl, or aralkyl is optionally substituted with one or more substituents selected from the Substituent Group A; (c) —(C═O)—R⁵—(C═O)-0R⁵—(C═O)—NR⁵R⁶ —(C═S)—NHR⁵, or —SO₂—R⁷

Z is a bond, C₁₋₁₅alkylene, C₂₋₁₆alkenylene, or C₂₋₁₆alkynylene; wherein the alkylene, alkenylene, or alkynylene is optionally substituted with one or more substituents selected from the Substituent Group A;

and a substituent of the Substituent Group A is selected from: oxo, halogen, C₁₋₃ alkylenedioxy, nitro, cyano, optionally halogenated C₁₋₃ alkyl, optionally halogenated C₂₋₆ alkenyl, carboxy C₂₋₆ alkenyl, optionally halogenated C₂₋₆ alkynyl, optionally halogenated C₃₋₆ cycloalkyl, C₆₋₁₄ aryl, optionally halogenated C₁₋₆ alkoxy, C₁₋₆ alkoxy-carbonyl-C₁₋₆ alkoxy, hydroxy, C₆₋₁₄ aryloxy, C₇₋₁₆ aralkyloxy, mercapto, optionally halogenated C₁₋₃ alkylthio, C₆₋₁₄ arylthio, C₇₋₁₆aralkylthio, amino, mono-C₁₋₃ alkylamino, mono-C₆₋₁₄ arylamino, di-C₁₋₃alkylamino, di-C₆₋₁₄arylamino, formyl, carboxy, C₁₋₃ alkyl-carbonyl, C₃₋₆cycloalkyl-carbonyl, C₁₋₃alkoxycarbonyl,

C₇₋₁₄ aryl-carbonyl, C₇₋₁₆ aralkyl-carbonyl, C₆₋₁₄ aryloxy-carbonyl, C₇₋₁₆ aralkyloxycarbonyl, carbamoyl, thiocarbamoyl, mono-C₁₋₃ alkyl-carbamoyl, di-C₁₋₃alkyl-carbamoyl, C₆₋₁₄aryl-carbamoyl, C₁₋₃alkylsulfonyl, C₆₋₁₄ arylsulfonyl, C₁₋₃alkylsulfinyl, C₆₋₁₄arylsulfinyl, formylamino, C₁₋₃ alkyl-carbonylamino, C₆₋₁₄ aryl-carbonylamino, C₁₋₃ alkoxycarbonylamino, C₁₋₃ alkylsulfonylamino, C₆₋₁₄ arylsulfonylamino, C₁₋₃alkyl-carbonyloxy, C₆₋₁₄ aryl-carbonyloxy, C₁₋₆alkoxy-carbonyloxy, mono-C₁₋₃ alkyl-carbamoyloxy, di-C₁₋₃alkylcarbamoyloxy, C₆₋₁₄ aryl-carbamoyloxy, sulfo, sulfamoyl, sulfinamoyl and sulfenamoyl.

Compounds of Genus pII can be prepared according to the disclosure of U.S. Pat. No. 7,115,746. Genus pII is characterized by Formula (pII) or stereoisomers thereof, isotopically-enriched compounds thereof, prodrugs thereof, solvates thereof, and pharmaceutically acceptable salts thereof.

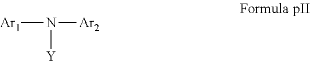

wherein:

definitions of variables are as provided in [00397] of WO 2019/071147.

Compounds of Genus pIII can be prepared according to the disclosure of U.S. Pat. No. 6,696,566. Genus pIII is characterized by Formula (pIII) or stereoisomers thereof, isotopically-enriched compounds thereof, prodrugs thereof, solvates thereof, and pharmaceutically acceptable salts thereof.

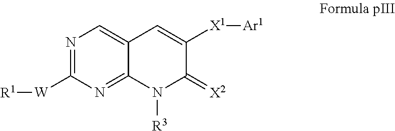

wherein:

definitions of variables are as provided in [00397] of WO 2019/071147.

Compounds of Genus pIV can be prepared according to the disclosure of US 2009/0042856. Genus pIV is characterized by Formula (pIV) or stereoisomers thereof, isotopically-enriched compounds thereof, prodrugs thereof, solvates thereof, and pharmaceutically acceptable salts thereof.

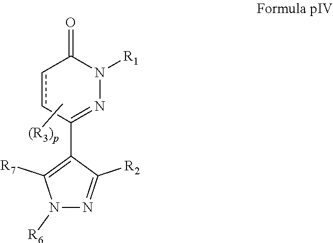

wherein:

definitions of variables are as provided in [00530] of WO 2019/071147.

Compounds of Genus pVI can be prepared according to the disclosure of U.S. Pat. No. 7,125,898. Genus pVI is characterized by Formula (pVI) or stereoisomers thereof, isotopically-enriched compounds thereof, prodrugs thereof, solvates thereof, and pharmaceutically acceptable salts thereof.

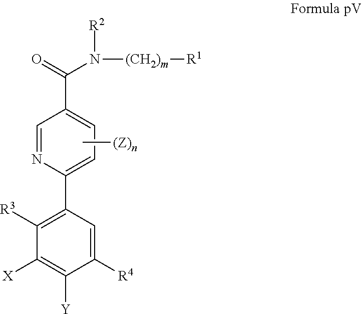

wherein:

definitions of variables are as provided in [00719] of WO 2019/071147.

Compounds of Genus pVI can be prepared according to the disclosure of U.S. Pat. No. 7,582,652. Genus pVI is characterized by Formula (pVI) or stereoisomers thereof, isotopically-enriched compounds thereof, prodrugs thereof, solvates thereof, and pharmaceutically acceptable salts thereof.

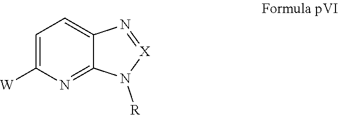

wherein:

definitions of variables are provided in [00769] of WO 2019/071147.

Compounds of Genus pVII can be prepared according to the disclosure of US 6,867,209. Genus pVII is characterized by Formula (pVII) or stereoisomers thereof, isotopically-enriched compounds thereof, prodrugs thereof, solvates thereof, and pharmaceutically acceptable salts thereof.

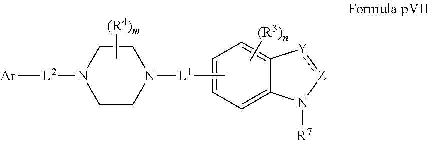

wherein:

definitions of variables are as provided in [00891] of WO 2019/071147.

Compounds of Genus pVIII can be prepared according to the disclosure of U.S. Pat. No. 6,319,921. Genus pVIII is characterized by Formula (pVIII) or stereoisomers thereof, isotopically-enriched compounds thereof, prodrugs thereof, solvates thereof, and pharmaceutically acceptable salts thereof.

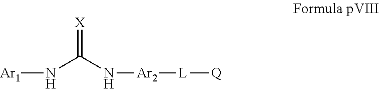

wherein:

definitions of variables are as provided in [00908] of WO 2019/071147.

Compounds of Genus pIX can be prepared according to the disclosure of U.S. Pat. Nos. 7,160,883, 7,462,616, and 7,759,343. Genus pIX is characterized by Formula (pIX) or stereoisomers thereof, isotopically-enriched compounds thereof, prodrugs thereof, solvates thereof, and pharmaceutically acceptable salts thereof.

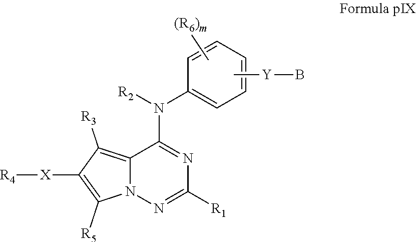

wherein:

definitions of variables are as provided in [001071] of WO 2019/071147.

Compounds of Genus pX can be prepared according to the disclosure of US 20050176775,. Genus pX is characterized by Formula (pX) or stereoisomers thereof, isotopically-enriched compounds thereof, prodrugs thereof, solvates thereof, and pharmaceutically acceptable salts thereof.

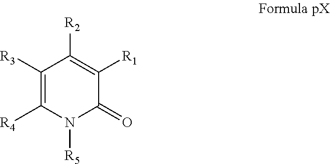

wherein:

definitions of variables are as provided in [001104] of WO 2019/071147.

Compounds of Genus pXI can be prepared according to the disclosure of U.S. Pat. Nos. 7,314,881, 7,323,472, and U.S. Pat. No. 8,058,282. Genus pXI is characterized by Formula (pXI) or stereoisomers thereof, isotopically-enriched compounds thereof, prodrugs thereof, solvates thereof, and pharmaceutically acceptable salts thereof.

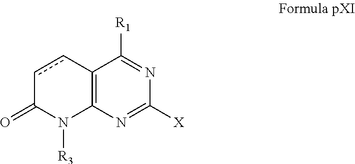

wherein:

definitions of variables are as provided in [001608] of WO 2019/071147.

Compounds of Genus pXII can be prepared according to the disclosure of U.S. Pat. No. 7,521,447. Genus pIII is characterized by Formula (pXII) or stereoisomers thereof, isotopically-enriched compounds thereof, prodrugs thereof, solvates thereof, and pharmaceutically acceptable salts thereof.

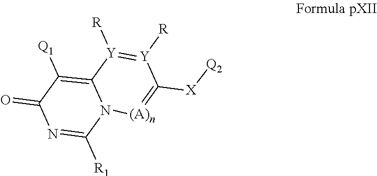

wherein:

definitions of variables are as provided in [001661] of WO 2019/071147.

Compounds of Genus pXIII can be prepared according to the disclosure of U.S. Pat. No. 7,521,447. Genus pXIII is characterized by Formula (pXIII) or stereoisomers thereof, isotopically-enriched compounds thereof, prodrugs thereof, solvates thereof, and pharmaceutically acceptable salts thereof.

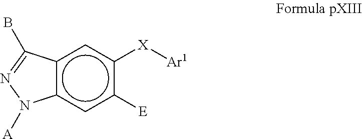

wherein:

definitions of variables are as provided in [001665] of WO 2019/071147.

The following are preferred p38 inhibitors:
1. The p38 inhibitors of general formula pl listed in paragraphs [00246] through [00294] of WO2019/071147.
2. The p38 inhibitors of general formula pII that is 2-(2,4-difluorophenyl)-6-(1-(2,6-difluoropheny)ureido)nicotinamide (“VX-702”).
3. The p38 inhibitors of general formula pill listed in paragraphs [00399] through [00496] of WO2019/071147.
4. The p38 inhibitors of general formula pIV listed in paragraphs [00532] through [00618] of WO2019/071147.
5. The p38 inhibitors of general formula pV listed in paragraphs [00721] through [00758] of WO2019/071147.
6. The p38 inhibitors of general formula pVI listed in paragraphs [00771] through [00885] of WO2019/071147.
7. The p38 inhibitors of general formula pVII listed in paragraphs [00893] through [00902] of WO2019/071147, preferably in paragraphs [00893] through [00900] and [00902].
8. The p38 inhibitors of general formula pVIII listed in paragraphs [00910] through [001068] of WO2019/071147, preferably in paragraph [001068].
9. The p38 inhibitors of general formula pIX listed in paragraphs [001072] through [001074] of WO2019/071147, preferably in paragraph [001074].
10. The p38 inhibitors of general formula pX listed in paragraphs [001106] through [001409] or [001412] through [001588] of WO2019/071147.

11. The p38 inhibitors of general formula pXI listed in paragraphs [001610] through [001644] of WO2019/071147.

12. The p38 inhibitors of general formula pXII listed in paragraphs [001662] and [001663] of WO2019/071147, preferably in [001663].

13. The p38 inhibitors of general formula pXIII listed in paragraphs [001667] through [001698] of WO2019/071147.

14. Losmapimod is a highly preferred p38 inhibitor.

15. Any of the p38 inhibitors listed in the above 1-14.

### Other CK1 Inhibitors and p38 Inhibitors

In certain embodiments, the inhibitors induce degradation of a target polypeptide, e.g. of p38 protein or of CK1 protein. For example, inhibitors include proteolysis targeting chimeras (PROTAC), which induce selective intracellular proteolysis of target proteins. PROTACs include functional domains, which may be covalently linked protein-binding molecules: one is capable of engaging an E3 ubiquitin ligase, and the other binds to the target protein meant for degradation. Recruitment of the E3 ligase to the target protein results in ubiquitination and subsequent degradation of the target protein by the proteasome. In particular preferred embodiments, a p38 inhibitor is a PROTAC that targets a p38 protein (e.g., p38-α and/or p38-β). In particular preferred embodiments, a CK1 inhibitor is a PROTAC that targets a CK1 protein (e.g. CK1δ and/or CK1ε). In particular preferred embodiments, a CK1 inhibitor is a PROTAC that targets a CK1 protein (e.g. CK1δ and/or CK1ε) and a p38 inhibitor is a PROTAC that targets a p38 protein (e.g., p38-α and/or p38-β).

### Composition

In a further aspect, the invention provides a composition comprising at least one CK1 inhibitor, and a pharmaceutically acceptable excipient, for use according to the invention. Such a composition is referred to herein as a composition for use according to the invention. Preferred compositions for use according to the invention are pharmaceutical compositions. In preferred embodiments, the composition for use according to the invention is formulated for oral, sublingual, parenteral, intravascular, intravenous, subcutaneous, or transdermal administration, optionally for administration by inhalation; preferably for oral administration. More features and definitions of administration methods are provided in the section on formulation and administration.

Preferred compositions comprise at least two distinct inhibitors, one of which is a CK1 inhibitor and the other being an agent that inhibits myogenic fusion and/or differentiation. Preferred such agents are defined elsewhere herein.

Other preferred compositions comprise at least one single agent that is a CK1 inhibitor and that is also a p38 inhibitor. Preferred such inhibitors are described elsewhere herein.

### Formulation and Administration

The compositions comprising the compounds as described above, can be prepared as a medicinal or cosmetic preparation or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube (referred to as enteral administration). A “dietary supplement” shall mean a product that is intended to supplement the human diet and is typically provided in the form of a pill, capsule, tablet or like formulation. By way of example, but not limitation, a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including, but not limited to, food bars, beverages, powders, cereals, cooked foods, food additives and candies; or other functional foods designed to promote health or to prevent or halt the progression of a degenerative disease associated with DUX4 expression or activity.

The subject compositions thus may be compounded with other physiologically acceptable materials that can be ingested including, but not limited to, foods. In addition or alternatively, the compositions for use as described herein may be administered orally in combination with (the separate) administration of food.

The compositions may be administered alone or in combination with other pharmaceutical or cosmetic agents and can be combined with a physiologically acceptable carrier thereof. In particular, the compounds described herein can be formulated as pharmaceutical or cosmetic compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21^{st} edition (2005) and 22^{nd} edition (2012), incorporated herein by reference.

It is known that many molecules that inhibit CK1 can also inhibit p38. p38 mitogen-activated protein kinases are a class of mitogen-activated protein kinases (MAPKs) that are responsive to stress stimuli, such as cytokines, ultraviolet irradiation, heat shock, and osmotic shock, and are involved in cell differentiation, cytokine secretion, apoptosis and autophagy. Persistent activation of the p38 MAPK pathway in muscle satellite cells (muscle stem cells) due to ageing is known to impair muscle regeneration. In preferred embodiments, the CK1 inhibitor is also a p38 inhibitor.

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/m icroparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For injection, the CK1 inhibitors and combinations and compositions for use according to the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Oral and parenteral administration may be used where the CK1 inhibitors and combinations and compositions for use are formulated by combining them with pharmaceutically acceptable carriers well known in the art, or by using them as a food additive. Such strategies enable the CK1 inhibitors and combinations and compositions for use according to the invention to be formulated as tablets, pills, dragées, capsules, liquids, gels, syrups, slurries, suspensions and the like, for oral ingestion by a subject to be treated. Preparations or pharmacological preparations for oral use may be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Dragée cores are provided with suitable coatings. For this purpose, concentrated sugar solutions may be used, which may optionally contain gum arabic, talc, PVP, carbopol gel, polyethylene glycol, and/or titanium dioxide, lacquer solution, and suitable organic solvents or solvent mixtures. Polymethacrylates can be used to provide pH-responsive release profiles so as to pass the stomach. Dyestuffs or pigments may be added to the tablets or dragée coatings for identification or to characterize different combinations of active CK1 inhibitor doses.

CK1 inhibitors and compositions which can be administered orally include push-fit capsules made of gelatin, as well as soft, sealed capsules made of gelatin and a plasticizer, such as glycerol or sorbitol. The push-fit capsules may contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the CK1 inhibitors and combinations and compositions for use according to the invention may be administered in the form of tablets or lozenges formulated in a conventional manner.

The CK1 inhibitors and combinations and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents. This formulation is preferred because it enables specific targeting of muscle tissue.

Compositions for parenteral administration include aqueous solutions of the compositions in water soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, before use.

The compositions or combinations for use according to the invention may also be formulated in rectal compositions such as suppositories or retention enemas, e.g., containing conventional suppository bases such as cocoa butter or other glycerides.

In addition to the formulations described previously, the CK1 inhibitors and combinations and compositions for use according to the invention may also be formulated as a depot preparation. Such long acting formulations may be administered by implantation (for example subcutaneously or intramuscularly) or by intramuscular injection. Thus, for example, they may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt. Examples of suitable polymeric materials are known to the person skilled in the art and include PLGA and polylactones such as polycaproic acid.

The compositions or combinations for use according to the invention also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compositions or combinations for use according to the invention may also be comprised in a transdermal patch. Preferred transdermal patches for use according to the invention are selected from single-layer drug-in-adhesive patch, or multi-layer drug-in-adhesive patch, or reservoir patch, or matrix patch, or vapour patch.

Compositions for use according to the invention include CK1 inhibitors and combinations and compositions wherein the active ingredients are contained in an amount effective to achieve their intended purposes. More specifically, a therapeutically effective amount means an amount of compound effective to prevent, stabilize, alleviate, revert, or ameliorate causes or symptoms of disease, or prolong the survival, mobility, or independence of the subject being treated. Determination of a therapeutically effective amount is within the capability of those skilled in the art, especially in light of the detailed disclosure provided herein. For any CK1 inhibitors and combinations and compositions used in the invention, the therapeutically effective amount or dose can be estimated initially from cell culture assays, for example as exemplified herein. Dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics” Ch. 1 p. 1). The amount of CK1 inhibitors and compositions administered will, of course, be dependent on the subject being treated, on the subject's weight, the severity of the affliction, the manner of administration and the judgment of the prescribing physician.

A composition or combination for use according to the invention may be supplied such that a CK1 inhibitor for use according to the invention and one or more of the other components as defined herein are in the same container, either in solution, in suspension, or in powder form. A composition for use according to the invention may also be provided with all components provided separately from one another, for example to be mixed with one another prior to administration, or for separate or sequential administration. For example, a composition can comprise a container comprising a CK1 inhibitor, and a separate container comprising a p38 inhibitor. Conversely, a composition can also comprise a container comprising in the same container both a CK1 inhibitor and a p38 inhibitor. Various packaging options are possible and known to the ones skilled in the art, depending, among others, on the route and mechanism of administration. In light of the methods of administration described above, the invention provides a casein kinase 1 inhibitor for use according to the invention, or a combination for use according to the invention, or a composition for use according to the invention, characterized in that it is administered orally, sublingually, intravascularly, intravenously, subcutaneously, transdermally, or optionally by inhalation; preferably orally.

An “effective amount” of a CK1 inhibitor or combination or composition is an amount which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations.

The “effective amount” of that may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 0.01 to 2000 mg, or about 0.01 to 1000 mg, or about 0.01 to 500 mg, or about 5 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 200 mg. In preferred embodiments, the invention provides a casein kinase 1 inhibitor for use according to the invention, or a composition for use according to the invention, characterized in that it is administered to a subject in an amount ranging from 0.1 to 1500 mg/day, preferably from 0.1 to 1000 mg/day, more preferably from 0.1 to 400 mg/day, still more preferably from 0.25 to 150 mg/day, such as about 100 mg/day.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

The total daily dose for children is preferably at most 200 mg. More preferably the total daily dose is about 0.1 to 200 mg, about 1 to 200 mg, about 5 to 200 mg about 20 to 200 mg about 40 to 200 mg, or about 50 to 200 mg. Preferably, the total daily dose for children is about 0.1 to 150 mg, about 1 to 150 mg, about 5 to 150 mg about 10 to 150 mg about 40 to 150 mg, or about 50 to 150 mg. More preferably, the total daily dose is about 5 to 100 mg, about 10 to 100 mg, about 20 to 100 mg about 30 to 100 mg about 40 to 100 mg, or about 50 to 100 mg. Even more preferably, the total daily dose is about 5 to 75 mg, about 10 to 75 mg, about 20 to 75 mg about 30 to 75 mg about 40 to 75 mg, or about 50 to 75 mg.

Alternative examples of dosages which can be used are an effective amount of the compounds for use according to the invention within the dosage range of about 0.1 μg /kg to about 300 mg/kg, or within about 1.0 μg /kg to about 40 mg/kg body weight, or within about 1.0 μg/kg to about 20 mg/kg body weight, or within about 1.0 μg /kg to about 10 mg/kg body weight, or within about 10.0 μg /kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

Compounds or compositions for use according to the present invention may be administered in a single daily dose, or the total daily dosage may be administered in divided dosage of two, three or four times daily.

In a preferred embodiment of the invention, “subject”, “individual”, or “patient” is understood to be an individual organism, preferably a vertebrate, more preferably a mammal, even more preferably a primate and most preferably a human.

In a further preferred embodiment of the invention, the human is an adult, e.g. a person that is 18 years or older. In addition, it is herein understood that the average weight of an adult person is 62 kg, although the average weight is known to vary between countries. In another embodiment of the invention the average weight of an adult person is therefore between about 50-90 kg. It is herein understood that the effective dose as defined herein is not confined to subjects having an average weight. Preferably, the subject has a BMI (Body Mass Index) between 18.0 to 40.0 kg/m², and more preferably a BMI between 18.0 to 30.0 kg/m².

Alternatively, the subject to be treated is a child, e.g. a person that is 17 years or younger. In addition, the subject to be treated may be a person between birth and puberty or between puberty and adulthood. It is herein understood that puberty starts for females at the age of 10-11 years and for males at the age of 11-12 year. Furthermore, the subject to be treated may be a neonate (first 28 days after birth), an infant (0-1 year), a toddler (1-3 years), a preschooler (3-5 years); a school-aged child (5-12 years) or an adolescent (13-18 years).

To maintain an effective range during treatment, the CK1 inhibitor or combination or composition may be administered once a day, or once every two, three, four, or five days. However preferably, the compound may be administered at least once a day. Hence in a preferred embodiment, the invention pertains to a casein kinase 1 inhibitor for use according to the invention, or a composition for use according to the invention, characterized in that it is administered to a subject 4, 3, 2, or 1 times per day or less, preferably 1 time per day. The total daily dose may be administered as a single daily dose. Alternatively, the compound is administered at least twice daily. Hence, the compound as defined herein may be administered once, twice, three, four or five times a day. As such, the total daily dose may be divided over the several doses (units) resulting in the administration of the total daily dose as defined herein. In a preferred embodiment, the compound is administered twice daily. It is further understood that the terms “twice daily”, “bid” and “bis in die” can be used interchangeable herein.

In a preferred embodiment, the total daily dose is divided over several doses per day. These separate doses may differ in amount. For example for each total daily dose, the first dose may have a larger amount of the compound than the second dose or vice versa. However preferably, the compound is administered in similar or equal doses. Therefore in a most preferred embodiment, the compound is administered twice daily in two similar or equal doses.

In a further preferred embodiment of the invention, the total daily dose of the compound as defined herein above is administered in at least two separate doses. The interval between the administration of the at least two separate doses is at least about 0.5, 1, 2, 3, 4, 5, 6, 7, 8, 9, 10, 11 or 12 hours, preferably the interval between the at least two separate doses is at least about 4, 5, 6, 7, 8, 9, 10, 11 or 12 hours and more preferably the interval between the at least two separate doses is at least about 8, 9, 10, 11 or 12 hours.

### Use

In one aspect of the invention, the use is provided of either a CK1 inhibitor according to the invention, or of a composition according to the invention, or of a combination according to the invention. Said use is for the treatment of a disease or condition associated with DUX4 expression of a subject in need thereof, and comprises administration to the subject of an effective dose of a CK1 inhibitor or combination or composition according to the invention, wherein the CK1 inhibitor or combination or composition are as defined earlier herein.

In one embodiment of this aspect, the use is provided of either a CK1 inhibitor according to the invention, or of a composition according to the invention, or of a combination according to the invention. Said use is for the treatment of muscular dystrophy or cancer in a subject in need thereof, and comprises administration to the subject of an effective dose of a CK1 inhibitor or composition or combination according to the invention, wherein the CK1 inhibitor or composition or combination are as defined earlier herein. Further features and definitions are preferably as defined elsewhere herein, particularly for diseases or conditions to be treated.

### Method

One aspect of the invention provides an in vivo, in vitro, or ex vivo method for reducing DUX4 expression, the method comprising the step of contacting a cell with a CK1 inhibitor as defined earlier herein, or with a composition or combination as defined earlier herein. One related aspect of the invention provides an in vivo, in vitro, or ex vivo method for promoting myogenic fusion and/or differentiation, the method comprising the step of contacting a cell with a CK1 inhibitor as defined earlier herein, or with a composition or combination as defined earlier herein. Preferably, said method is for treating a disease or condition associated with DUX4 expression, such as a muscular dystrophy or cancer, most preferably said disease or condition is facioscapulohumeral muscular dystrophy (FSHD). The method preferably comprises use as defined earlier herein. Preferred methods comprise contacting a cell with a CK1 inhibitor composition as defined earlier herein. In the context of the invention, contacting a cell with a CK1 inhibitor or a combination or a composition can comprise adding such a CK1 inhibitor or combination or composition to a medium in which a cell is cultured. Contacting a cell with a CK1 inhibitor or a combination or a composition can also comprise adding such a CK1 inhibitor, combination, or composition to a medium, buffer, or solution in which a cell is suspended, or which covers a cell. Other preferred methods of contacting a cell comprise injecting a cell with a CK1 inhibitor, combination, or composition, or exposing a cell to a material comprising a CK1 inhibitor, combination, or composition according to the invention. Further methods for administration are defined elsewhere herein. Preferred cells are cells known to express DUX4, cells suspected of expressing DUX4, or cells known to be affected by a disease or condition as defined earlier herein.

In one embodiment of this aspect, the method is an in vitro method. In a further embodiment of this aspect, the method is an ex vivo method. In a further embodiment of this aspect, the method is an in vivo method. In a preferred embodiment of this aspect, the method is an in vitro or an ex vivo method.

Within the embodiments of this aspect, the cell may be a cell from a sample obtained from a subject. Such a sample may be a sample that has been previously obtained from a subject. Within the embodiments of this aspect, samples may have been previously obtained from a human subject. Within the embodiments of this aspect, samples may have been obtained from a non-human subject. In a preferred embodiment of this aspect, obtaining the sample is not part of the method according to the invention.

In preferred embodiments, the method according to the invention is a method for reducing DUX4 expression in a subject in need thereof, the method comprising the step of administering an effective amount of a CK1 inhibitor as defined earlier herein, or a composition as defined earlier herein, or a combination as defined earlier herein. In more preferred embodiments, the method is for the treatment of a disease or condition associated with DUX4 expression, preferably a muscular dystrophy or cancer, most preferably said disease or condition is facioscapulohumeral muscular dystrophy (FSHD). Further features and definitions are preferably as defined elsewhere herein.

### General Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

When a structural formula or chemical name is understood by the skilled person to have chiral centers, yet no chirality is indicated, for each chiral center individual reference is made to all three of either the racemic mixture, the pure R enantiomer, and the pure S enantiomer. Preferred isomers are tautomers and stereoisomers.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use. CK1 inhibitors or compositions according to this invention are preferably for use in methods or uses according to this invention.

Throughout this application, expression is considered to be the transcription of a gene into functional mRNA, leading to a polypeptide such as an enzyme or transcription factor or for example DUX4 polypeptide. A polypeptide can assert an effect or have an activity. In this context, increased or decreased expression of a polypeptide can be considered an increased or decreased level of mRNA encoding said polypeptide, an increased or decreased level or amount of polypeptide molecules, or an increased or decreased total activity of said polypeptide molecules. Preferably, an increased or decreased expression of a polypeptide results in an increased or decreased activity of said polypeptide, respectively, which can be caused by increased or decreased levels or amounts of polypeptide molecules. More preferably, a reduction of DUX4 expression is a reduction of transcription of a DUX4 gene, destabilisation or degradation of DUX4 mRNA, reduction of the amount of DUX4 polypeptide molecules, reduction of DUX4 polypeptides molecule activity, destabilisation or degradation of DUX4 polypeptide, or combinations thereof. A destabilized mRNA leads to lower expression of its encoded polypeptide, possibly it cannot lead to such expression. A degraded mRNA is destroyed and cannot lead to expression of its encoded polypeptide. A destabilized polypeptide asserts less of an effect or has lower activity than the same polypeptide that has not been destabilized, possibly it asserts no effect or has no activity. A destabilized polypeptide can be denatured or misfolded. A degraded polypeptide is destroyed and does not assert an effect or have an activity.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 1% of the value.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### SHORT DESCRIPTION

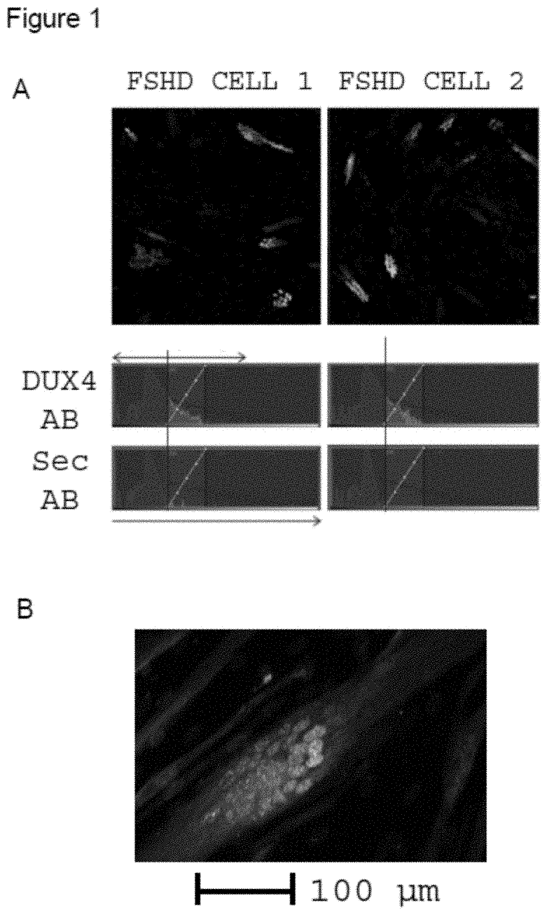

FIG. 1—(A): Illustration of a DUX4 immunocytochemistry staining in FSHD myotubes from 2 different donors after 3 days of differentiation. DUX4-positive nuclei clusters are clearly stained, while DUX4-negative nuclei are not stained. The histograms show the intensity of the immunofluorescent signals (increasing intensity on the X-axis) after staining with the DUX4 and secondary antibody (top) or the secondary antibody alone (bottom); the arrows on top show the background signal (leftward arrow) or specific DUX4 signal (rightward arrow); (B): Illustration of a DUX4-stained FSHD myotube after 3 days of differentiation. The dotted pattern results from the applied filter settings to deplete the background from the secondary antibody control. Note that the threshold settings prohibit detection of the weaker DUX4 signal in the nuclei more distant from the sentinel nucleus.

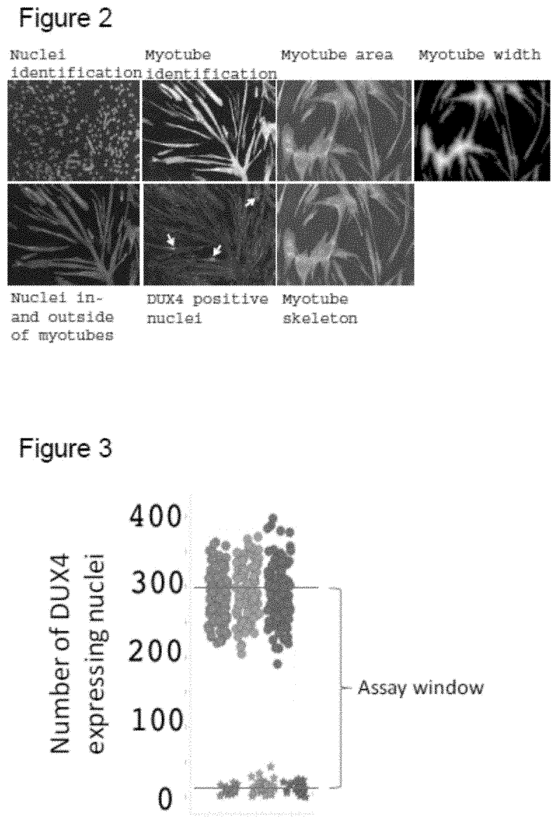

FIG. 2—Script-based image analysis includes nuclei identification, myotube identification, detection of nuclei inside or outside myotube borders (used to calculate fusion index), DUX4 positive nuclei and clusters, myotube area, myotube width, and myotube skeleton length.

FIG. 3—Validation of the primary screening assay format in 384-well format. Three independent experiments are shown, illustrating the assay window obtained using script-based quantification of the number of DUX4-expressing nuclei in differentiating primary myotubes after 3 days in differentiation medium. The assay window is defined by the DUX4 signal and the background signal of the secondary antibody (representing the signal in total absence of DUX4).

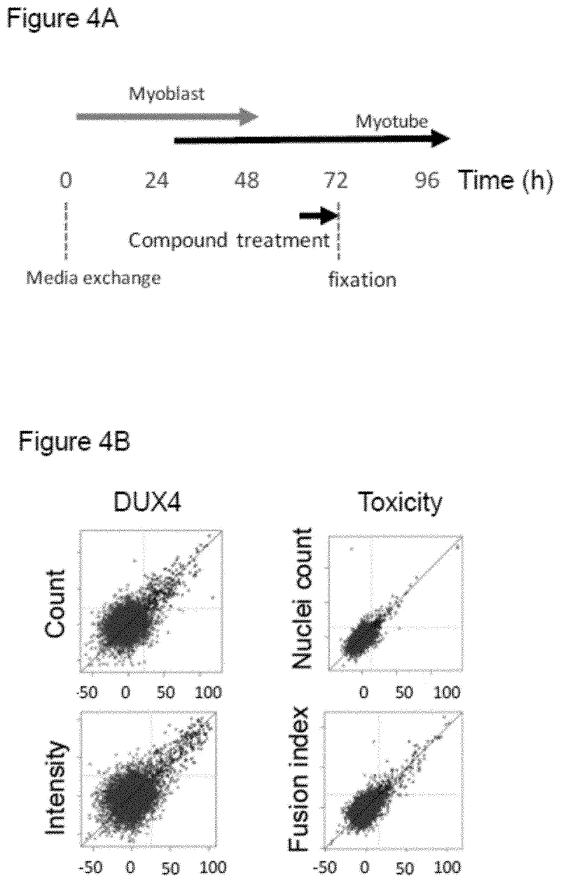

FIG. 4–(A): Schematic representation of the screening assay protocol. Myoblasts were seeded at day -1 and medium was changed to differentiation medium at day zero. Cells were allowed to differentiate for 3 days. Compounds were added 15 h prior to fixation. (B): Correlation of duplicated results from primary screening of an annotated compound library using 2 different readouts for DUX4 expression (Number of DUX4-positive nuclei and DUX4 intensity) and 2 different readouts to monitor potential toxicity (fusion index, nuclei count). Hit calling thresholds (high stringency) are indicated by a dashed line, and the upper right quadrants contain the hit compounds for the different readouts. Axes of the scatter plots are symmetrical.

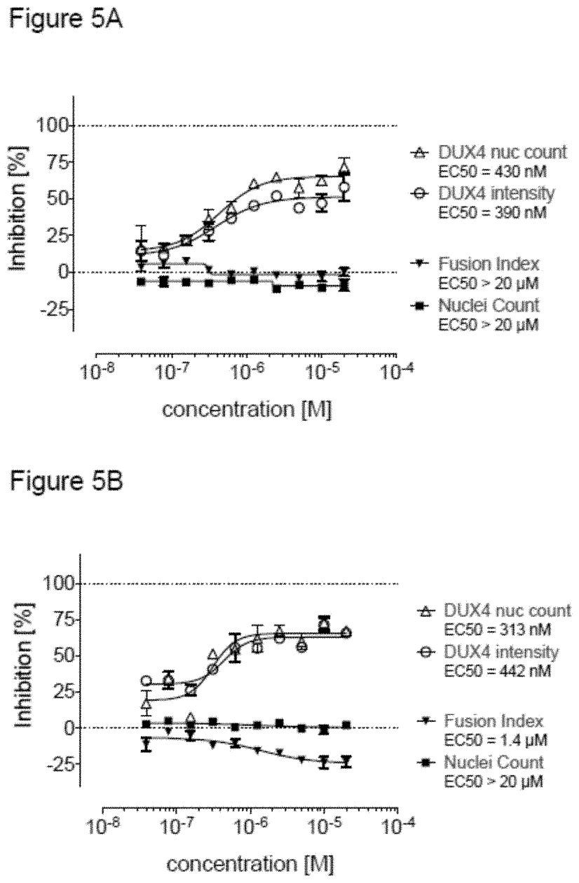

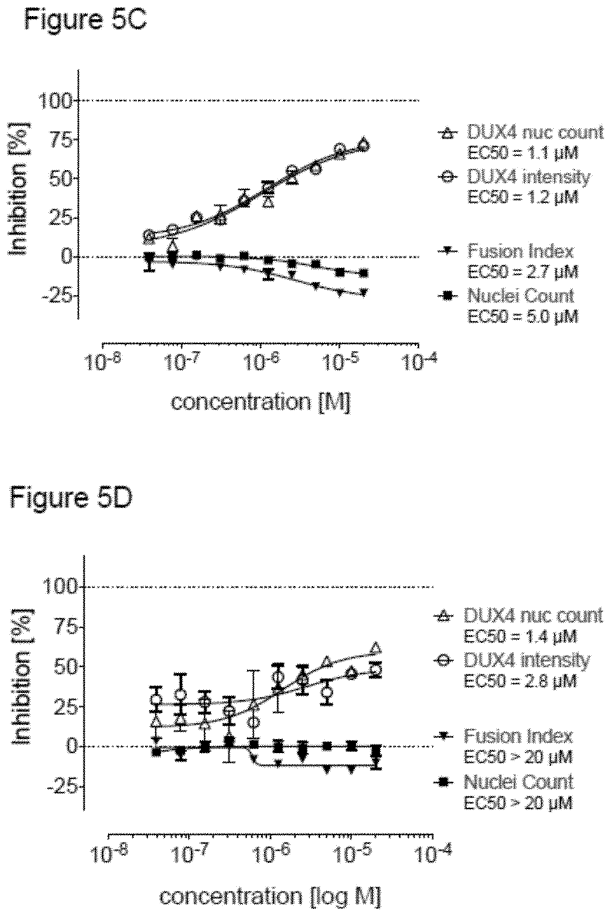

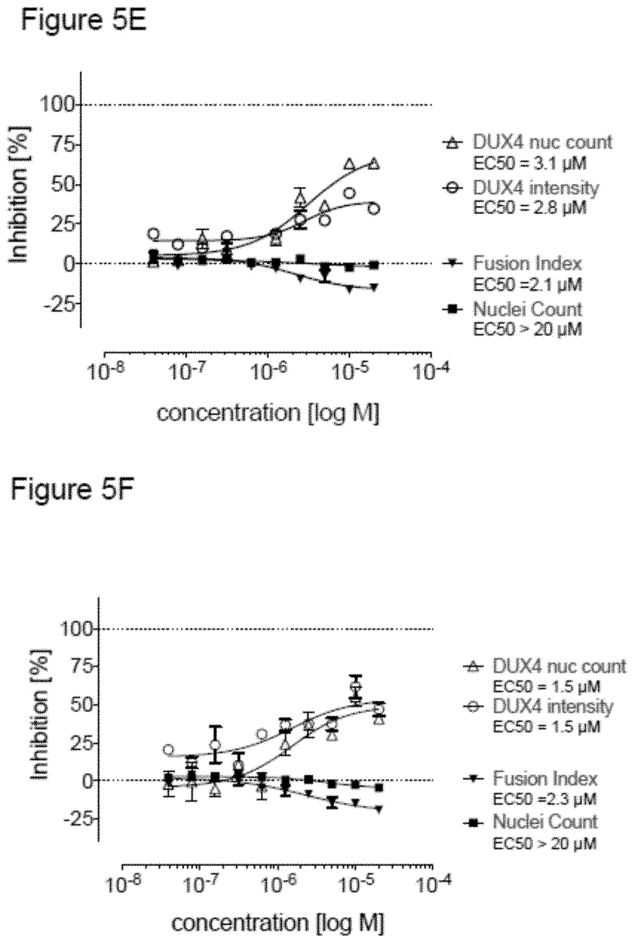

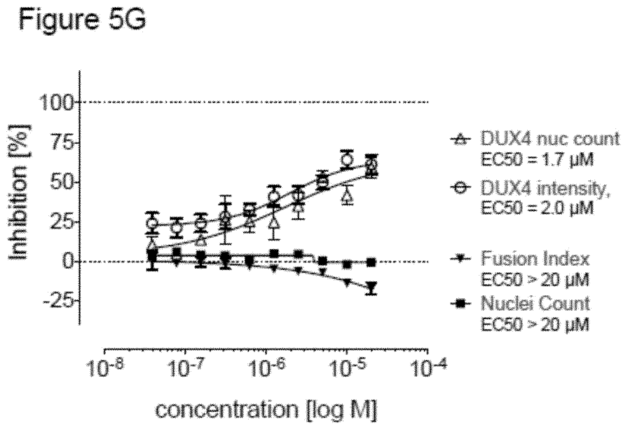

FIG. 5—Concentration-response curves for various CK1 inhibitors for the different readouts. The DUX4 nuclei count, DUX4 intensity, fusion index, and total nucleus count were measured after 15 hour of compound exposure. (A): results for PF-670462; (B): results for PF-5006739; (C): results for compound 3; (D): results for compound 4; (E): results for compound 5; (F): results for compound 6; (G): results for compound 7; Structural formulae are shown in example 5.

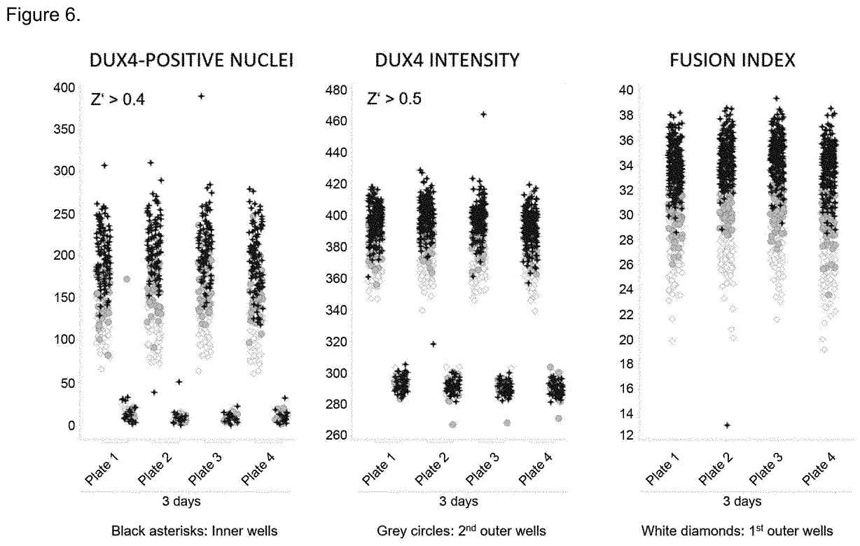

FIG. 6—Scatter plot of one assay validation experiment for the readouts DUX4 nuclei count (left), DUX4 intensity (middle) and fusion index (right). Primary FSHD myotubes were grown in proliferation medium after which the medium was replaced with differentiation medium and the cells were allowed to differentiate for 3 days. The readouts were assessed as explained in example 2. The most outer wells are indicated by white diamonds, the second outer wells with grey circles and all inner wells with black asterisk. It is clear from the graphs that the fusion index in the most outer wells is lower compared to the inner wells. Also the DUX4 readouts are lower in the outer wells, illustrating that reduction of the fusion index implies a risk of obtaining a false positive readout.

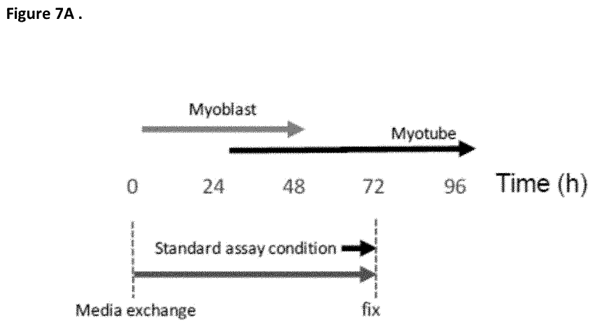

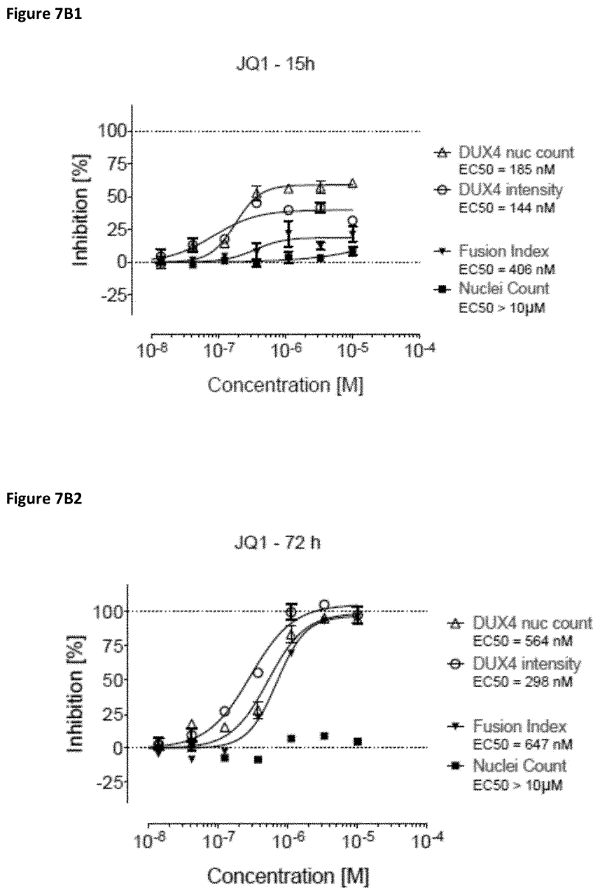

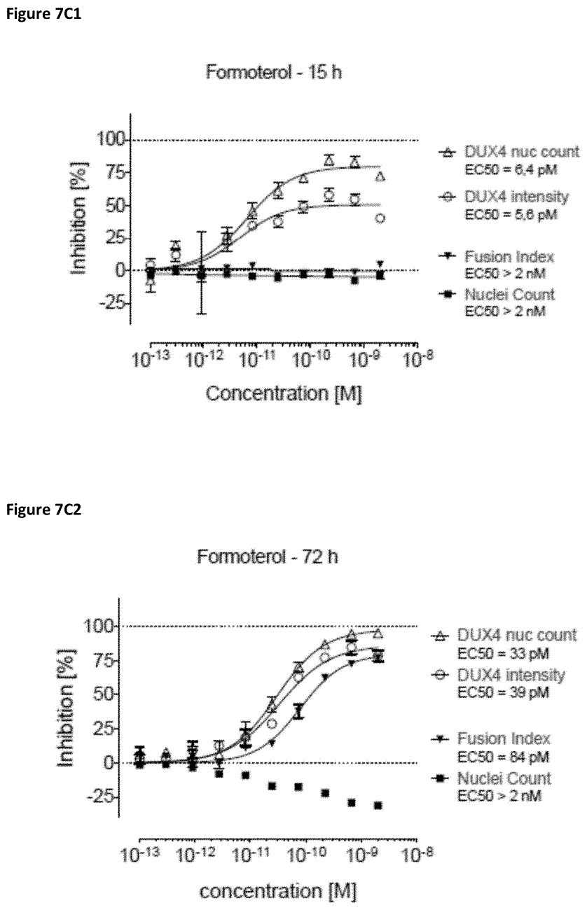

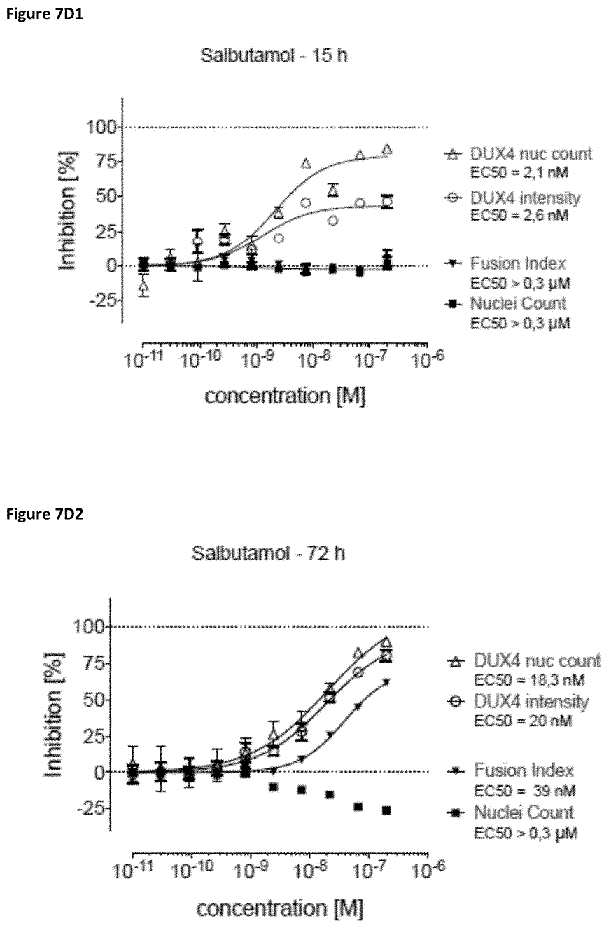

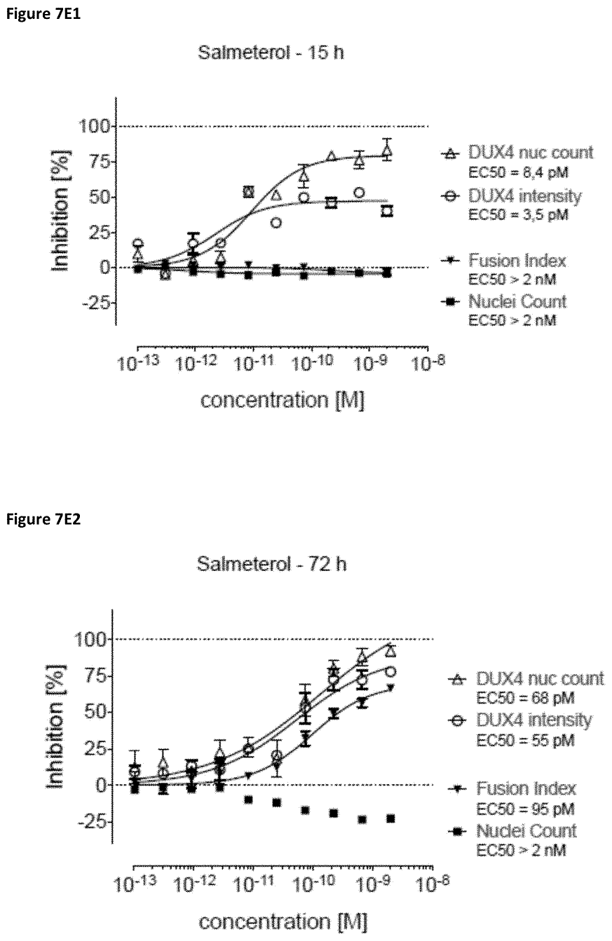

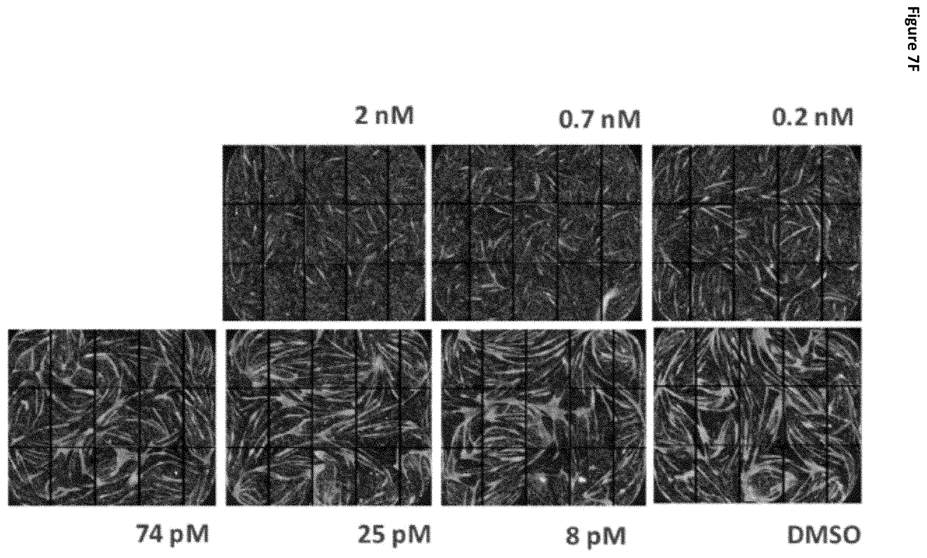

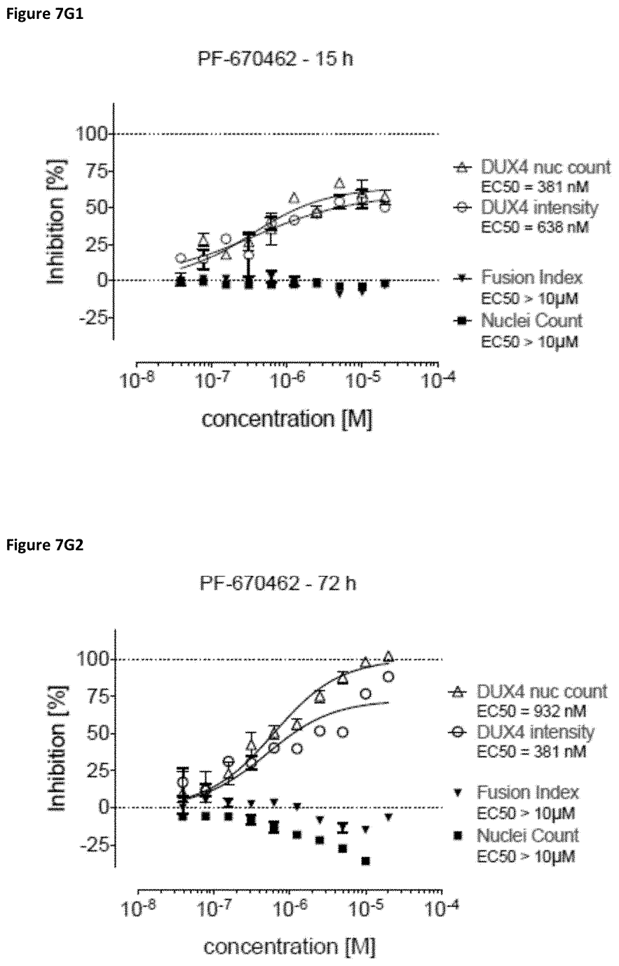

FIG. 7—(A): Schematic representation of the assay protocol. Myoblasts were seeded at day -1 and medium was changed to differentiation medium at day zero. Cells were allowed to differentiate for 3 days. Compounds were added for 15 h or 72 h prior to fixation. For the 15 h treatment, compounds are administered when differentiation already progressed significantly. In case of 72 h treatment, compounds were incubated during the full differentiation phase. The other panels show concentration-response curves for a BET inhibitors (B) or for beta2 adrenoreceptor agonists (C, D, E, F) for the different readouts. DUX4 nuclei count, DUX4 intensity, fusion index, and total nuclei count were assessed after 15 h or after 72 h of treatment. (B): (+)JQ1; (C): formoterol; (D): salbutamol; (E): salmeterol; (F): micrographs of myotubes after 72 hours in differentiation medium while exposed to the a beta2 adrenoreceptor agonist (formoterol); (G): results for both 15 hour and 72 hour exposure to a CK1 inhibitor (PF-670462).

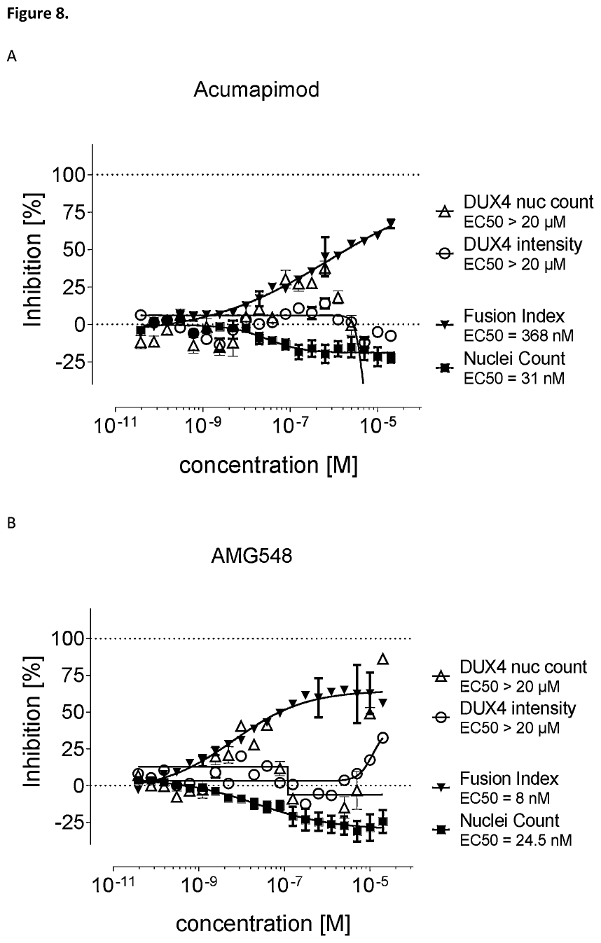

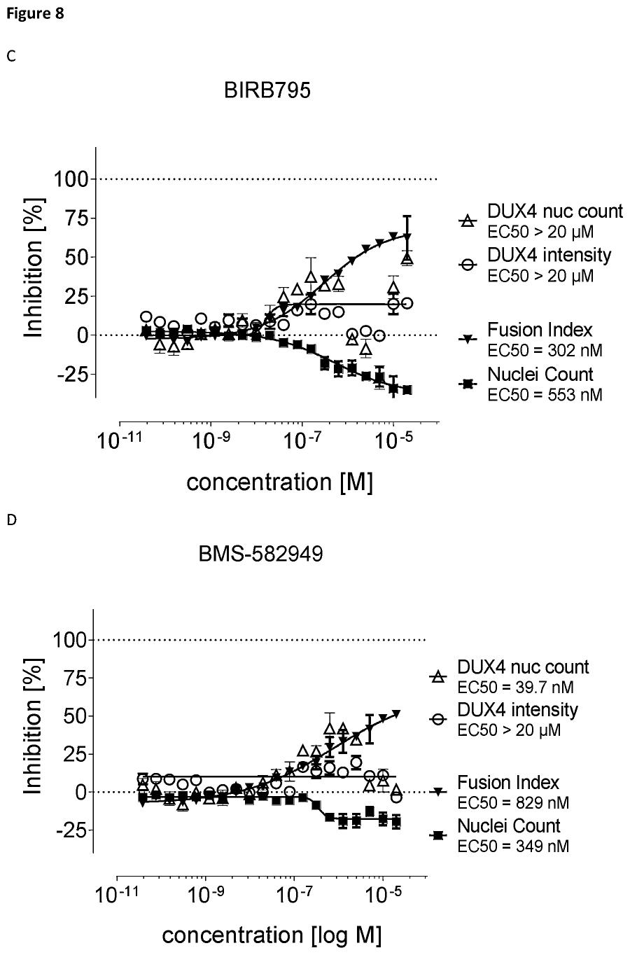

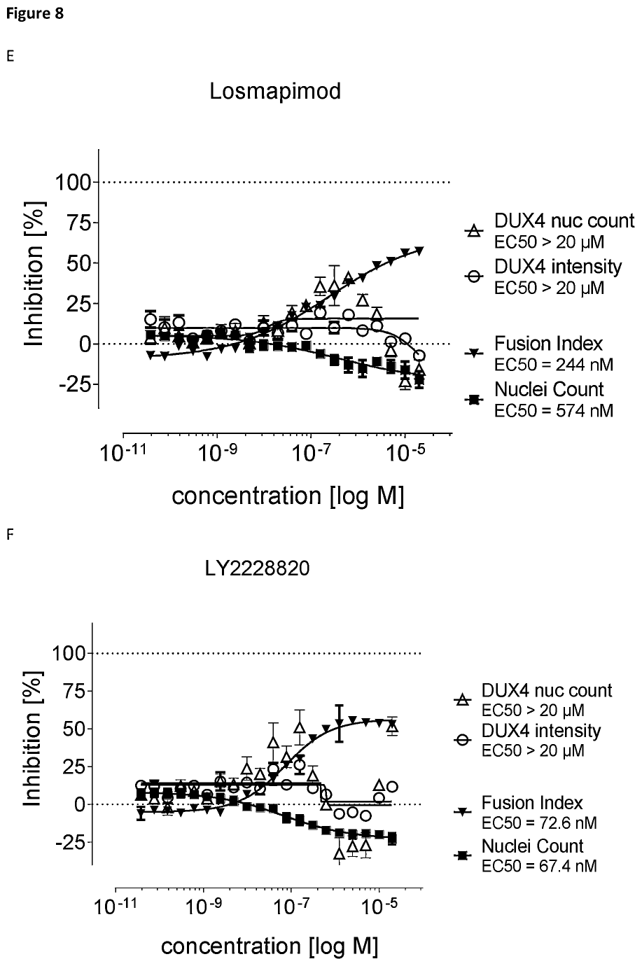

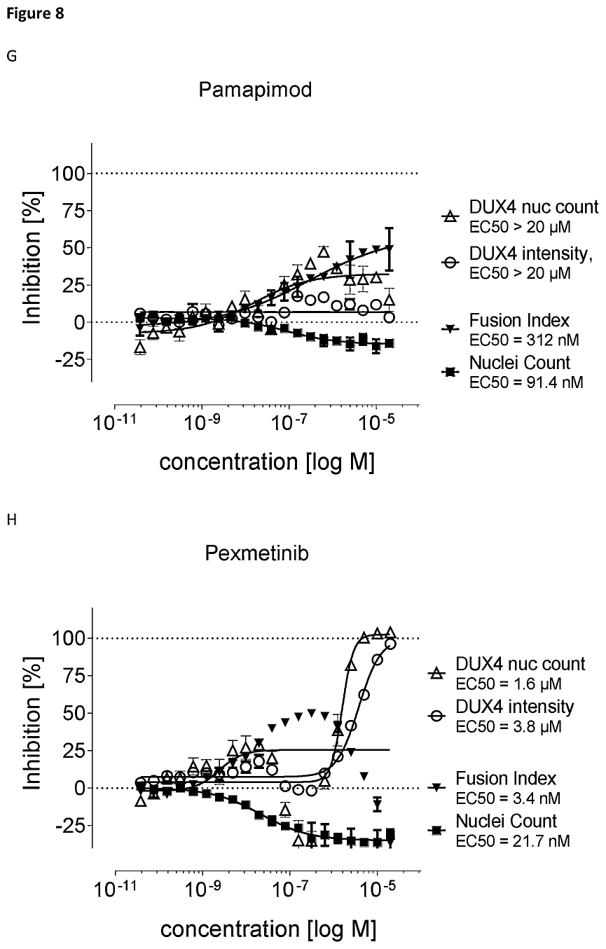

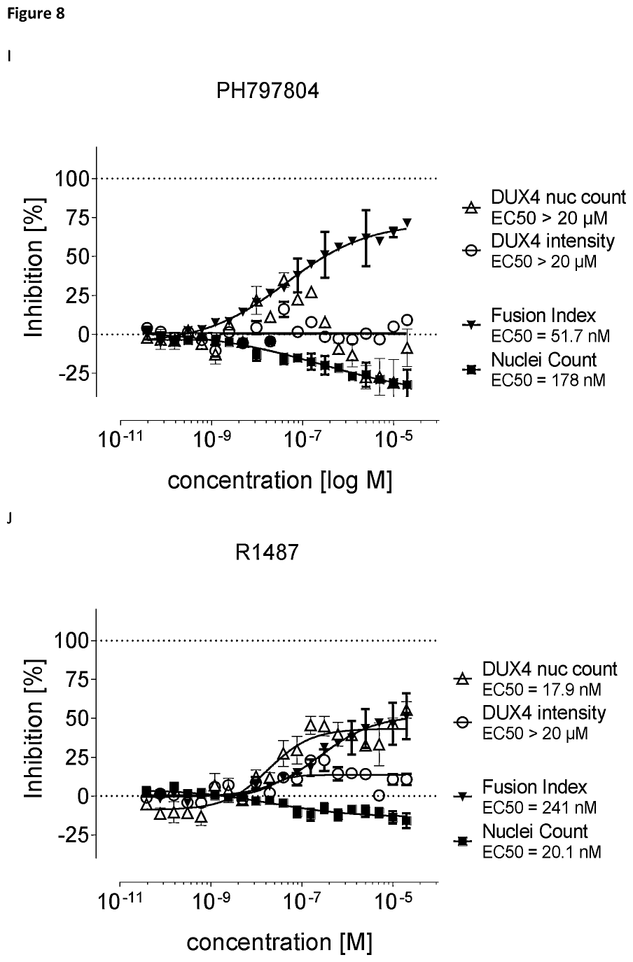

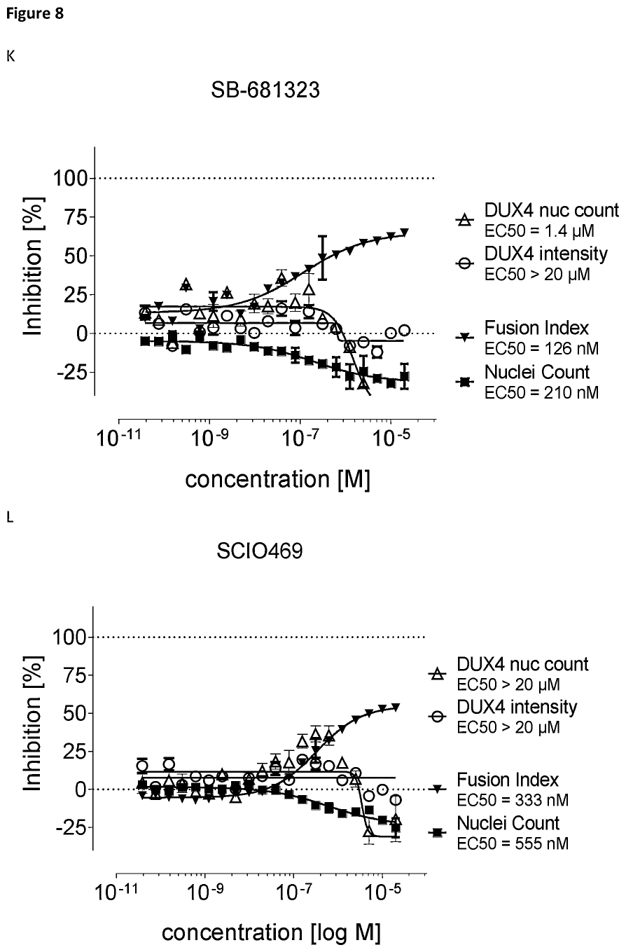

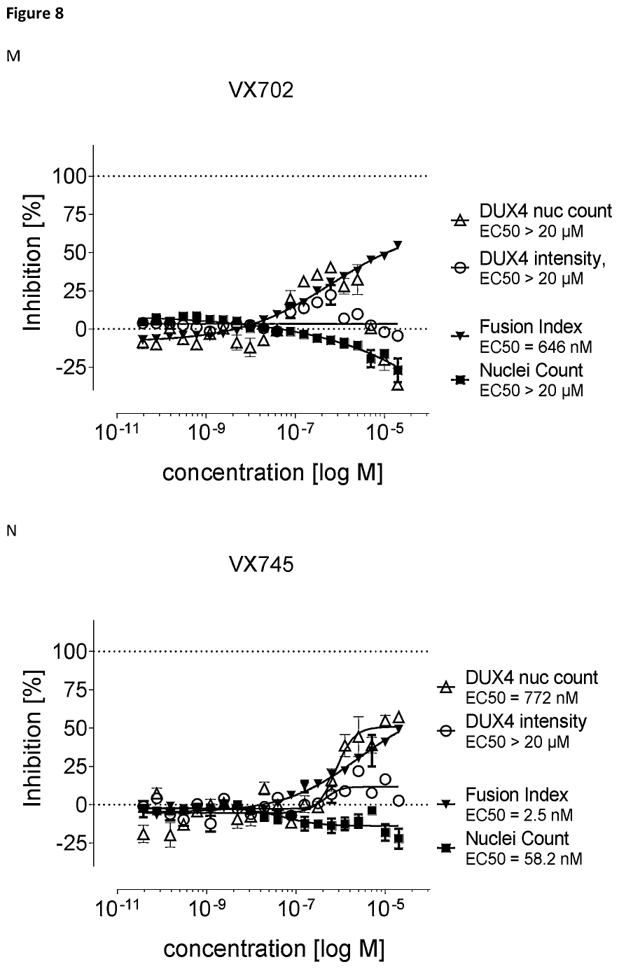

FIG. 8—Concentration-response curves (n=3) for various p38 inhibitors for the different readouts in a primary FSHD cell line. The DUX4 nuclei count, DUX4 intensity, fusion index, and total nucleus count were measured after 72 hour of compound exposure. (A): results for Acumapimod; (B): results for AMG548; (C): results for BIRB795; (D): results for BMS-582949; (E): results for losmapimod; (F): results for LY2228820; (G): results for pamapimod; (H): results for pexmetinib; (I): results for PH797804; (J): results for R1487; (K): results for SB-681323; (L): results for SCIO469; (M): results for VX702; (N): results for VX745.

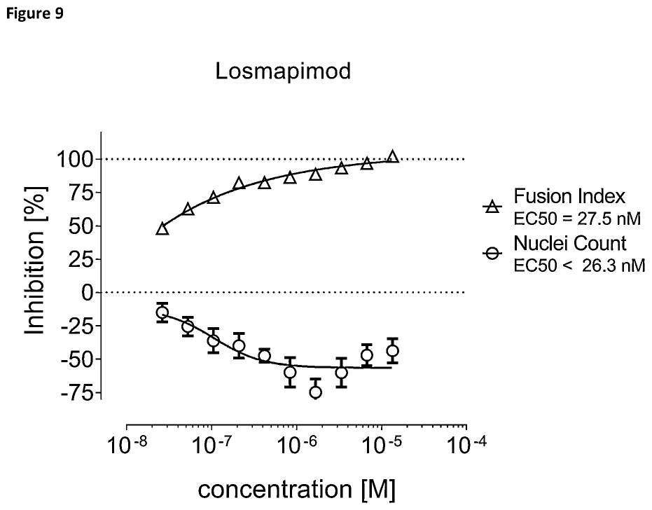

FIG. 9—Concentration-response curves for the p38 inhibitor losmapimod for the fusion and cell count readout in a primary cell from a healthy donor. The fusion index is strongly inhibited by losmapimod.

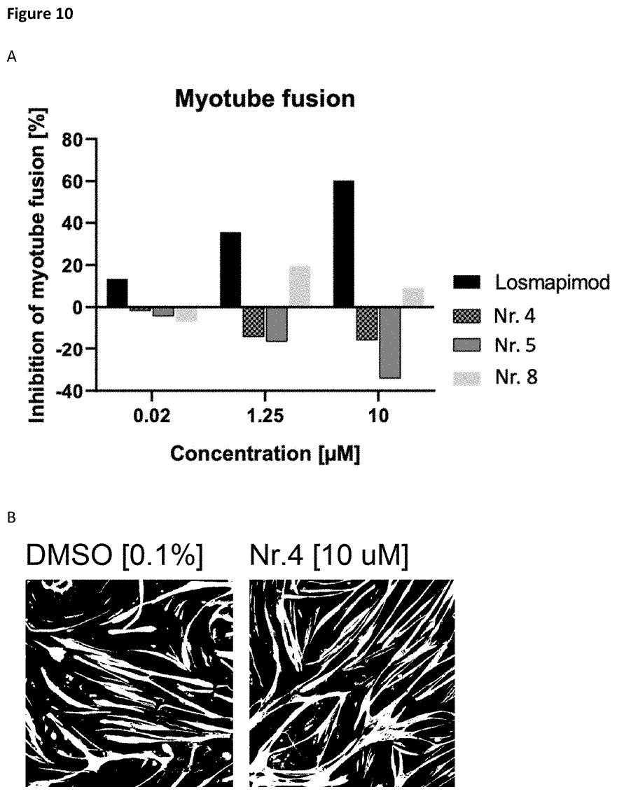

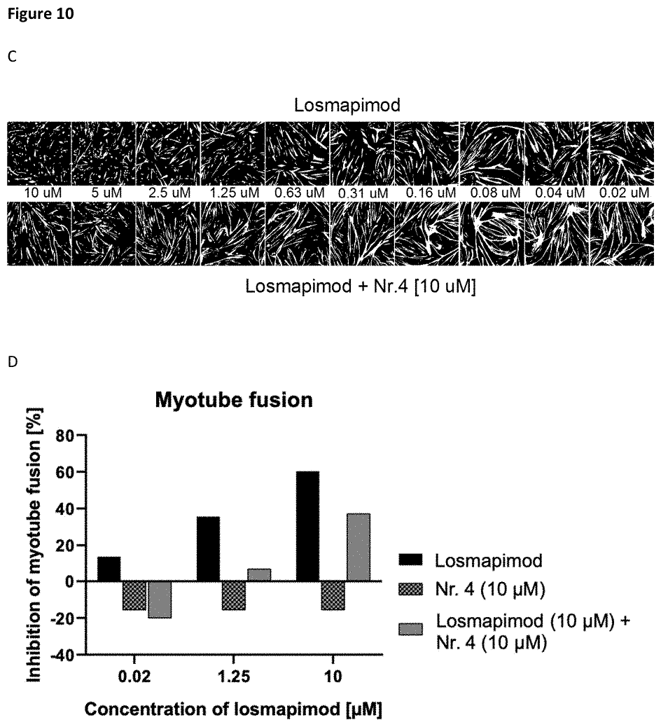

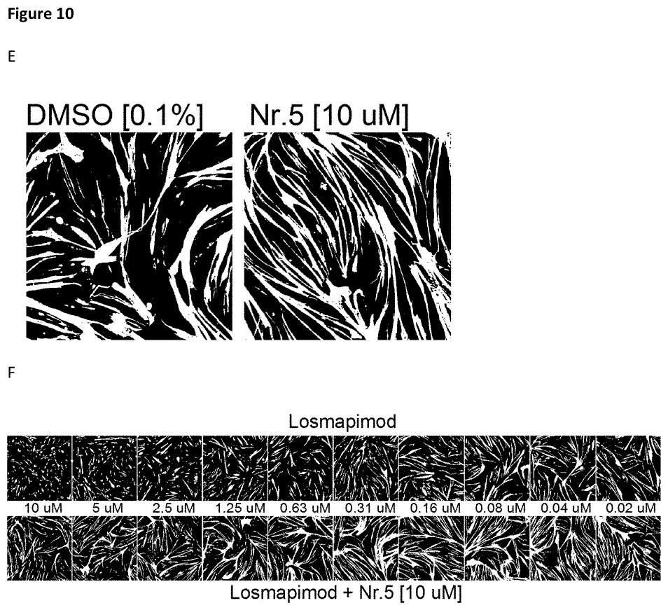

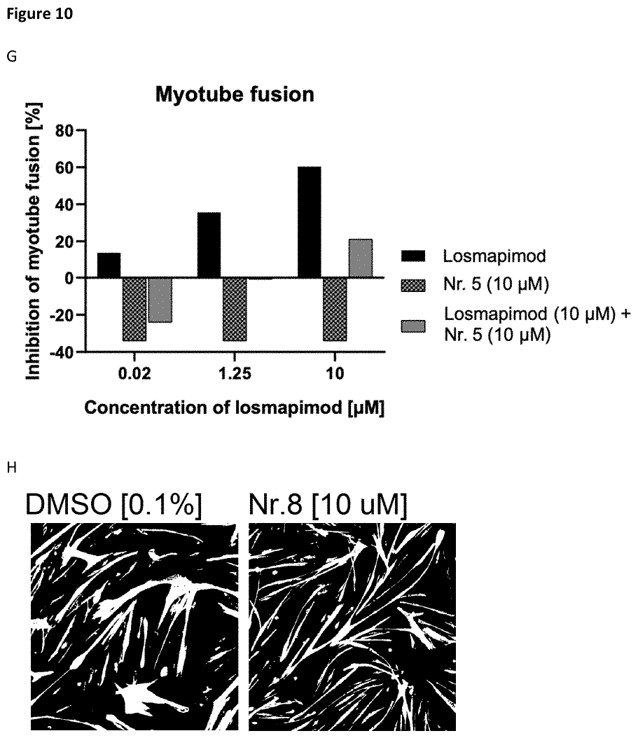

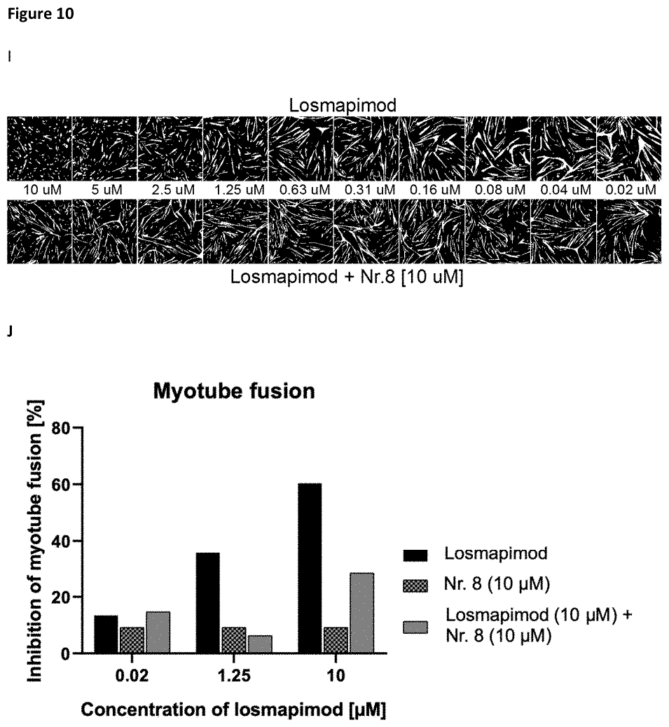

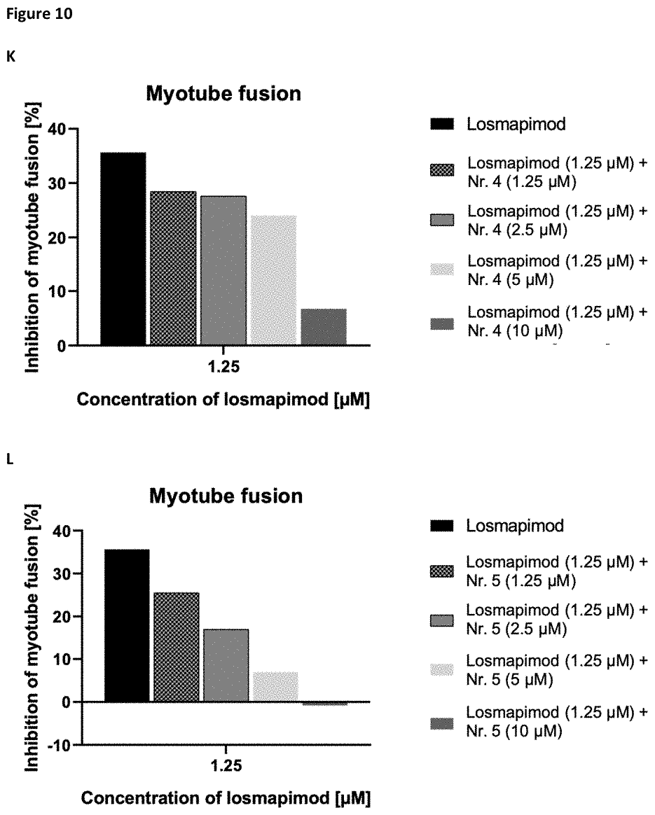

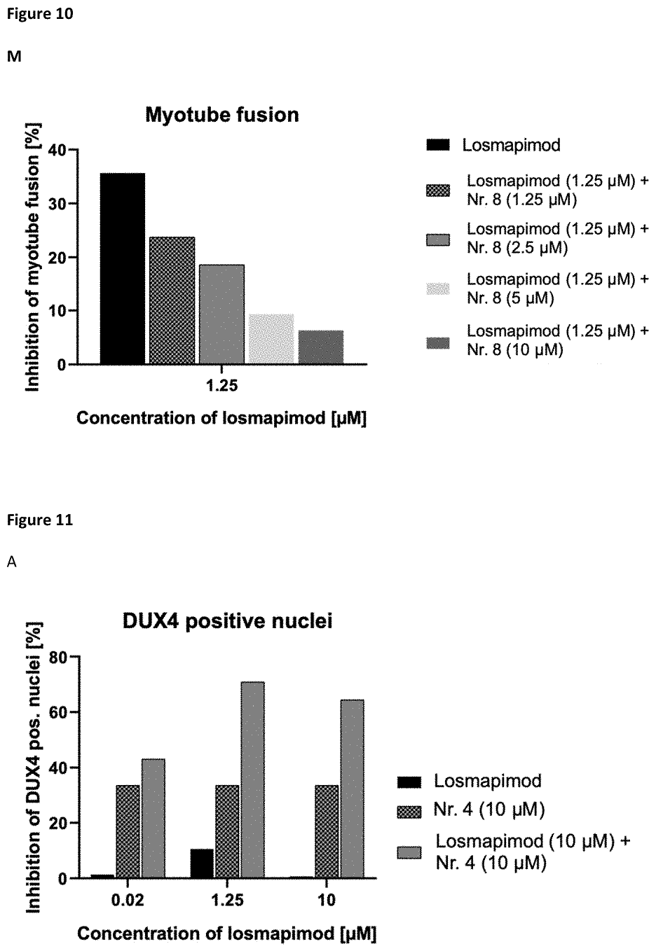

FIG. 10—Experiments have been performed in the standard assay in primary FSHD cells, with 72 h of compound treatment. (A): Concentration-dependent effect on the fusion index by increasing concentrations of the p38 inhibitor losmapimod or CK1 inhibitors Nr. 4, Nr 5 or Nr 8; (B): Microscopic images of primary FSHD cells in the standard assay after 72 h of treatment with solvent or the CK1 inhibitor Nr.4; (C): Microscopic images of primary FSHD cells in the standard assay after 72 h of treatment with different concentrations of losmapimod in the absence (top) or presence (bottom) of the CK1 inhibitor Nr.4; (D): Concentration-dependent effect on the fusion index by increasing concentrations of the p38 inhibitor losmapimod either in the absence or presence of the CK1 inhibitor Nr.4. The effect of the CK1 inhibitor alone is shown for comparison; (E): Microscopic images of primary FSHD cells in the standard assay after 72 h of treatment with solvent or the CK1 inhibitor Nr.5; (F): Microscopic images of primary FSHD cells in the standard assay after 72 h of treatment with different concentrations of losmapimod in the absence (top) or presence (bottom) of the CK1 inhibitor Nr.5; (G): Concentration-dependent effect on the fusion index by increasing concentrations of the p38 inhibitor losmapimod either in the absence or presence of the CK1 inhibitor Nr.5. The effect of the CK1 inhibitor alone is shown for comparison; (H): Microscopic images of primary FSHD cells in the standard assay after 72 h of treatment with solvent or the CK1 inhibitor Nr.8; (I): Microscopic images of primary FSHD cells in the standard assay after 72 h of treatment with different concentrations of losmapimod in the absence (top) or presence (bottom) of the CK1 inhibitor Nr.8; (J): Concentration-dependent effect on the fusion index by increasing concentrations of the p38 inhibitor losmapimod either in the absence or presence of the CK1 inhibitor Nr.8. The effect of the CK1 inhibitor alone is shown for comparison; (K): The effect of a fixed concentration of losmapimod (1.25 uM) either in the absence or presence of increasing concentrations of the CK1 inhibitor Nr.4; (L): The effect of a fixed concentration of losmapimod (1.25 uM) either in the absence or presence of increasing concentrations of the CK1 inhibitor Nr.5; (M): The effect of a fixed concentration of losmapimod (1.25 uM) either in the absence or presence of increasing concentrations of the CK1 inhibitor Nr.8

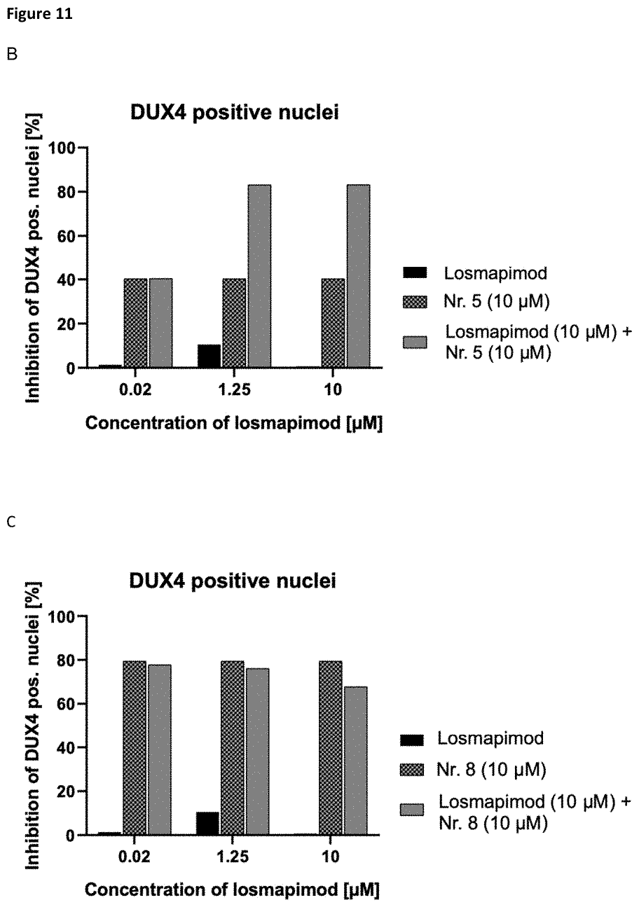

FIG. 11—Experiments have been performed in the standard assay in primary FSHD cells, with 72 h of compound treatment. (A): Concentration-dependent effect on the DUX4 readout by increasing concentrations of the p38 inhibitor losmapimod either in the absence or presence of the CK1 inhibitor Nr.4; (B): Concentration-dependent effect on the DUX4 readout by increasing concentrations of the p38 inhibitor losmapimod either in the absence or presence of the CK1 inhibitor Nr.5; (C): Concentration-dependent effect on the DUX4 readout by increasing concentrations of the p38 inhibitor losmapimod either in the absence or presence of the CK1 inhibitor Nr.8.

### EXAMPLES

### Example 1—Primary FSHD Muscle Cells Express DUX4 in a Small Fraction of Myonuclei

The inventors succeeded in establishing a sensitive DUX4 detection method in primary myotubes and used this to build a high-content assay for quantitative assessment of endogenous DUX4 expression. The method was developed into a validated phenotypic screening platform for automated detection and quantification of endogenous DUX4 expression. Mechanisms underlying DUX4 repression may involve many interacting proteins, favouring such a phenotypic approach. Furthermore, it is pathway/target independent (and thus not hypothesis-driven) and provides additional information on cell toxicity or interference with muscle differentiation.

Significant differences in the levels of DUX4 expression between cells obtained from different donors have been reported. Therefore, muscle cell lines derived from different donors were thoroughly characterised and an optimal cell line was selected for primary screening. MyoD staining of myoblasts confirmed solid myogenicity of all cell lines (Rudnicki et al., 1993; cell 75(7):1351-9). After optimisation of parameters, a DUX4 detection procedure was established that could be applied in a screening assay which resulted in the expected DUX4 pattern in FSHD cells, but not in myotubes from healthy donors. As shown in FIG. 1, this included a nuclear DUX4 localization, with only few positive cells, and an intensity gradient through DUX4-positive nuclear clusters, as also described by Rickard et al., (2015, DOI: 10.1093/hmg/ddv315).

### Example 2—Screening Assay to Ddentify DUX4 Repression

A quantitative assay readout was developed based on script-based image analysis. Cells were stained according to example 1, also using DAPI to detect myonuclei and an antibody against myosin heavy chain (MHC) to visualize the formation of myotubes. To analyse the images, an automated script was developed, enabling the detection of nuclei, myotube borders and DUX4 signals, with the script also detecting artefacts to reduce false positive signals. The script enabled multiple validated readouts including the number of DUX4 positive nuclei and nuclei clusters, the fusion index, myotube area, myotube width and myotube skeleton length (see FIG. 2). Additionally, the total nuclei count was included as a measure of cell loss or compound toxicity. The script was validated by evaluating endogenous DUX4 expression in the primary myotubes, and results were in line with literature values, with the number of DUX4 expressing nuclei being <0.5%.

The assay has been further matured to make it suitable for screening purposes. The assay quality was dependent on the donor cell line. The number of DUX4 positive nuclei was characteristic for each donor cell line, and was consistent between experiments. The best performing cell lines in terms of number of DUX4 expressing nuclei, reproducibility and Z-factor have been selected for miniaturization of the assay to a 384-well format, thus allowing for automated screening of large compound libraries. A cell line with 2 D4Z4 repeats was selected for the primary screening, while a cell line with 6 D4Z4 repeats was selected for later validation. The primary screening assay had a Z-factor of 0.6, which represents an excellent assay (Zhang et al., 1999, doi:10.1177/108705719900400206 ; see FIG. 3).

A compound library containing approximately 5000 annotated compounds was screened in the high-content assay. For this purpose, primary myoblasts were seeded in 384 well plates after which the growth medium was replaced with differentiation medium. After 3 days of differentiation, cells were treated with library compounds (in duplicate on different screening plates) for 15 h, after which they were fixed and stained with antibodies against DUX4, antibodies against myosin heavy chain (MHC), and with DAPI (4′,6-diamidino-2-phenylindole). Script-based analysis provided readouts for DUX4 expression (count of DUX4-positive nuclei or DUX4 intensity) and for potential toxicity (fusion index and nuclei count). Results are shown in FIG. 4. The majority of the approximately 200 hits was confirmed in an experiment using the same assay and 5 replicates. These compounds were selected for further concentration-response profiling.

Half of these hits were validated using RT-PCR. Based on mRNA expression of DUX4 and the downstream target genes Trim43 & ZScan4, using housekeeping genes hGUSB, GAPDH, hRPL27 as a reference, a very good correlation between DUX4 repression in the immunocytochemistry assay (protein level) and the RT-PCR assay (mRNA level) was observed. This suggests that the vast majority of the hits have an upstream mode of action, i.e. they act by inhibiting the expression of DUX4 (as opposed to accelerating degradation of DUX4).

RT-PCR was performed as described by Lemmers et al., (2010, DOI: 10.1126/science.1189044) using oligonucleotides ordered from Applied Biosystems (Foster City, USA), possibly as part of assay kits (for hGAPDH (app): AssaylD Hs02758991_g1; for hTRIM43(app): Assay ID Hs00299174_m1; for hMYH2_tv1-2(app): AssaylD Hs00430042_m1). Other oligonucleotides are shown in table 1.

TABLE 1
primers and probes for use in PCR

| | | SEQ |
|---|---|---|
| | | ID |
| Name | Sequence | NO: |
| hDUX4 forward | CCCGGCTGACGTGCAA | 1 |
| hDUX4 reverse | AGCCAGAATTTCACGGAAGAAC | 2 |
| hDUX4 probe | AGCTCGCTGGCCTCTCTGTGCC | 3 |
| hGUSB forward | TTCCCTCCAGCTTCAATGACA | 4 |
| hGUSB reverse | CCACACCCAGCCGACAA | 5 |
| hGUSB probe | AGGACTGGCGTCTGCGGCA | 6 |
| hRPL27 forward | TGTCCTGGCTGGACGCTACT | 7 |
| hRPL27 reverse | GAGGTGCCATCATCAATGTTCTT | 8 |
| hRPL27 probe | CGGACGCAAAGCTGTCATCGT | 9 |
| hZSCAN4 forward | AGGCAGGAATTGCAAAGACTTT | 10 |
| hZSCAN4 reverse | AATTTCATCCTTGCTGTGCTTTT | 11 |
| hZSCAN4 probe | TAGGATCTTTCACTCATGGCTGCAACCA | 12 |
| hMYOG forward | GCTCACGGCTGACCCTACA | 13 |
| hMYOG reverse | CACTGTGATGCTGTCCACGAT | 14 |
| hMYOG probe | CCCACAACCTGCACTCCCTCACCT | 15 |

### Example 3—CK1 Inhibitors Act as DUX4 Repressors

The validated assay was used for screening an annotated compound library containing approximately 5000 compounds, to identify novel mechanisms of action for DUX4 repression. This library contained compounds with annotated pharmacology, not only entailing the primary pharmacology of the compounds but also potential known polypharmacology. The primary screening achieved multiple hits, identifying compounds that reduced the number of DUX4 positive nuclei. Hits were further profiled by establishing concentration-response curves. By applying a bioinformatics approach on the screening and profiling dataset, the inventors surprisingly discovered that compounds with a CK1 annotation were significantly enriched in the phenotypically active compound population, i.e. in the group of compounds inducing a repression of DUX4. Interestingly, none of the original compounds with a CK1 annotation had CK1 as its primary pharmacological target, each having other high potency targets from other protein families. Thus the bioinformatics analysis was essential in identifying the association between CK1 and DUX4 repression.

Profiled compounds were annotated as being phenotypically active when they showed a concentration-dependent effect on DUX4 (inhibition or activation). Of these, compounds which showed inhibition of the fusion index or of the total number of nuclei by more than 10% were excluded unless the effect on these readouts was at least 5-fold less potent than the effect on DUX4. As such, from the 4790 unique compounds, 188 compounds were classified as being phenotypically active, 162 of which were DUX4 inhibitors.

For the phenotypically active compounds, the original target annotations were complemented with additional information that is publically available (literature, patent applications, supplier databases, etc.). All human proteins, and non-human orthologues where a mapping to the human proteome can be established, were considered. Each of the 4790 compounds was then evaluated against these target annotations, classifying the target as being active or inactive for a given compound. For the phenotypically active compounds, the annotated targets were classified as being active if the compound's potency on the target was 10 times the phenotypic potency, otherwise the target was classified as inactive. This analysis revealed that approximately 201 targets were associated with phenotypic activity at a False Discovery Rate of 0.05. An enrichment of compounds annotated as CK1 inhibitors was detected in the group of phenotypically active compounds.

### Example 4—CK1 Isoforms are Expressed in FSHD Primary Muscle Cells

To confirm target expression in both healthy and FSHD muscle cells, an RNA sequencing approach was followed to determine the expression of the different CK1 isoforms in primary myotubes from 4 different FSHD donors and from 4 different healthy donors. The results show expression of all CK1 isoforms, both in FSHD and in healthy muscle cells. The highest expression is of CK1 α, CK1 δ and CK1 ε (see table 2).

TABLE 2
expression of casein kinase 1 isoforms in 4 healthy primary cell lines, and in 4
FSHD primary cell lines as determined by RNA sequencing of differentiated myotubes

| | CSNK1A1 | CSNK1D | CSNK1E | CSNK1G1 | CSNK1G2 | CSNK1G3 |
|---|---|---|---|---|---|---|
| FSHD | 134 | 159.1 | 160.1 | 49.9 | 81.8 | 37.9 |
| FSHD | 122.5 | 138.4 | 136.8 | 4.2 | 79.1 | 32.7 |
| FSHD | 176.7 | 170.6 | 120.5 | 69.8 | 65.8 | 41.3 |
| FSHD | 118.2 | 134 | 105.6 | 41.8 | 63.5 | 38.1 |
| Healthy | 138.9 | 168.5 | 188 | 45.8 | 75.9 | 35.8 |
| Healthy | 143.3 | 174.1 | 200.7 | 49.6 | 81.8 | 36.3 |
| Healthy | 139.2 | 192.8 | 176.1 | 51.9 | 71.4 | 33.2 |
| Healthy | 119.1 | 132.4 | 122.4 | 40.6 | 65.9 | 40.1 |

### Example 5—Inhibition of CK1 Represses DUX4

The DUX4 repression of CK1 inhibitors was assayed following the protocol of Example 2, illustrated in FIG. 4A. Table 3 shows the structures of the CK1 inhibitors that are used in FIG. 5. Compounds were incubated with primary FSHD cells for 15 hours, as indicated by the arrow in FIG. 4A. Results are shown in FIG. 5, while table 3 shows half maximal effective concentrations (EC₅₀) values. Table 3 also shows determined IC₅₀ values in nM for CK1α, CK1δ, CK1ε, and p38α, denoted as CK1 a, d, e, and p38a, respectively.

TABLE 3
Exemplary CK1 inhibitors for use according to the invention, along
with half maximal effective concentrations (EC₅₀) for DUX4
repression obtained in the 15 h treatment protocol.

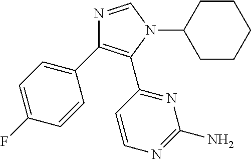 [SMILES: Nc1nccc(-c2c(-c3ccc(F)cc3)ncn2C2CCCCC2)n1]

PF-670462 (EC₅₀ 0.43 μM)
CK1 a: 320; d: 29.1; e: 99.8; p38a: 32.4

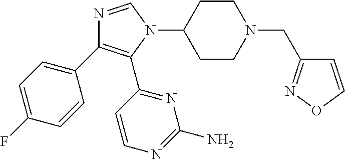 [SMILES: Nc1nccc(-c2c(-c3ccc(F)cc3)ncn2C2CCN(Cc3ccon3)CC2)n1]

PF-5006739 (EC₅₀ 0.31 μM)
CK1 a: 123; d: 19.8; e: 26.8; p38a: 74.3

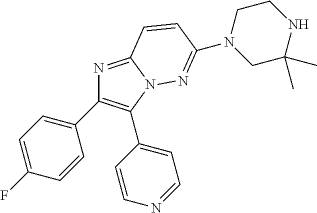 [SMILES: CC1(C)CN(c2ccc3nc(-c4ccc(F)cc4)c(-c4ccncc4)n3n2)CCN1]

Nr. 1 (EC₅₀ 0.04 μM)
CK1 a: 29.5; d: 18.5; e: 12.4; p38a: 13.2

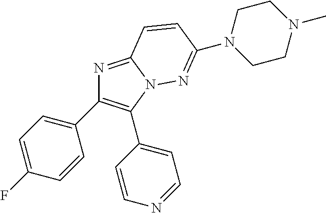 [SMILES: CN1CCN(c2ccc3nc(-c4ccc(F)cc4)c(-c4ccncc4)n3n2)CC1]

Nr. 2 (EC₅₀ 2 μM)

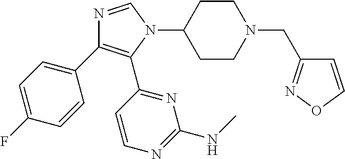 [SMILES: CNc1nccc(-c2c(-c3ccc(F)cc3)ncn2C2CCN(Cc3ccon3)CC2)n1]

Nr. 3 (EC₅₀ 1.1-1.4 μM)

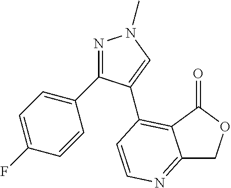 [SMILES: Cn1cc(-c2ccnc3c2C(=O)OC3)c(-c2ccc(F)cc2)n1]

Nr. 4 (EC₅₀ 1.4 μM)
CK1 a: 644; d: 33.1; e: 51.6; p38a: 569

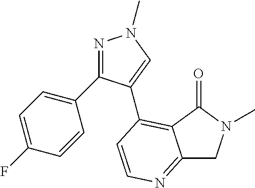 [SMILES: CN1Cc2nccc(-c3cn(C)nc3-c3ccc(F)cc3)c2C1=O]

Nr. 5 (EC₅₀ 1.9-3.1 μM)
CK1 a: 592; d: 30.7; e: 83.6; p38a: 1110

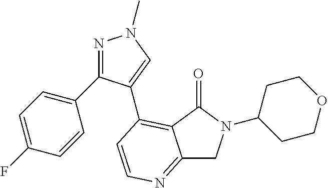 [SMILES: Cn1cc(-c2ccnc3c2C(=O)N(C2CCOCC2)C3)c(-c2ccc(F)cc2)n1]

Nr. 6 (EC₅₀ 1.5-2.6 μM)
CK1 a: 561; d: 18; e: 72.4; p38a: 677

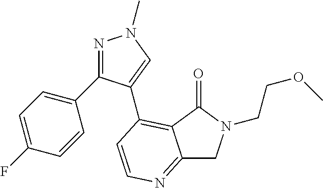 [SMILES: COCCN1Cc2nccc(-c3cn(C)nc3-c3ccc(F)cc3)c2C1=O]

Nr. 7 (EC₅₀ 1.7-4.5 μM)
CK1 a: 2590; d: 41.8; e: 92.1; p38a: 712

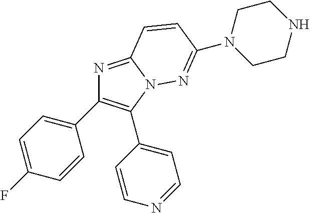 [SMILES: Fc1ccc(-c2nc3ccc(N4CCNCC4)nn3c2-c2ccncc2)cc1]

Nr. 8 (EC₅₀ 0.01-0.046 μM)
CK1 a: 22; d: 16.5; e: 9.41; p38a: 14.8

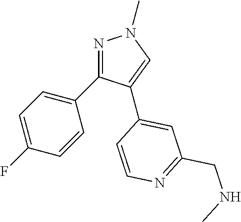 [SMILES: CNCc1cc(-c2cn(C)nc2-c2ccc(F)cc2)ccn1]

Nr. 9 (EC₅₀ 3.1-5.5 μM)
CK1 a: 1760; d: 57.7; e: 89; p38a: 3070

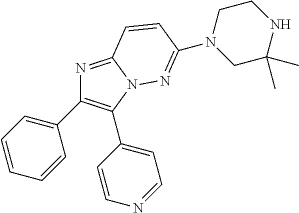 [SMILES: CC1(C)CN(c2ccc3nc(-c4ccccc4)c(-c4ccncc4)n3n2)CCN1]

Nr. 10

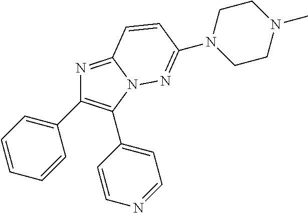 [SMILES: CN1CCN(c2ccc3nc(-c4ccccc4)c(-c4ccncc4)n3n2)CC1]

Nr. 11

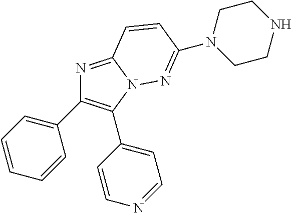 [SMILES: c1ccc(-c2nc3ccc(N4CCNCC4)nn3c2-c2ccncc2)cc1]

Nr. 12

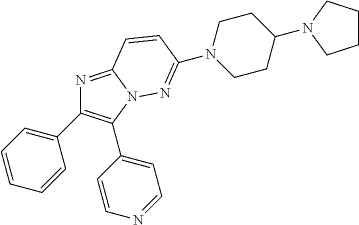 [SMILES: c1ccc(-c2nc3ccc(N4CCC(N5CCCC5)CC4)nn3c2-c2ccncc2)cc1]

Nr. 13

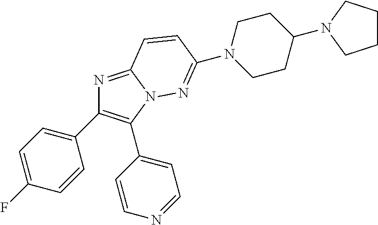 [SMILES: Fc1ccc(-c2nc3ccc(N4CCC(N5CCCC5)CC4)nn3c2-c2ccncc2)cc1]

Nr. 14

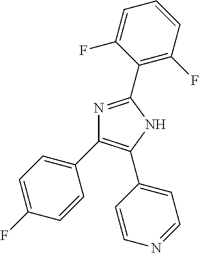 [SMILES: Fc1ccc(-c2nc(-c3c(F)cccc3F)[nH]c2-c2ccncc2)cc1]

Nr. 15 (EC₅₀ 0.71 μM)

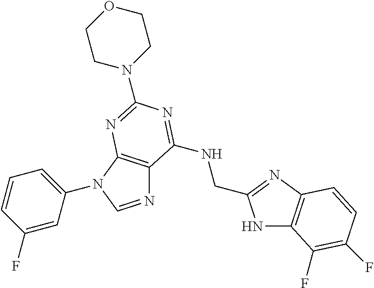 [SMILES: Fc1cccc(-n2cnc3c(NCc4nc5ccc(F)c(F)c5[nH]4)nc(N4CCOCC4)nc32)c1]

SR-3029 (EC₅₀ 0.05-0.12 μM)
CK1 a: 1000; d: 346; e: 38; p38a: >1000

Selected lead compounds were also tested in vivo, in a xenograft mouse model. For this purpose, human primary FSHD myoblasts were injected into the mouse Tibialis Anterior muscle. These human cells then differentiated into myotubes during which DUX4 is derepressed. A selected compound with good pharmacokinetic properties, ensuring exposure above the in vitro observed EC50, caused repression of the DUX4 mRNA expression in this xenograft animal model, as established by RT-PCR and histological examination.

### Example 6—Reduced Myotube Fusion Index Correlates With Reduced DUX4 Signals

In an assay validation experiment, the inventors identified that Interestingly, this directly reflected the DUX4 count readout from the assay, illustrating that small effects on fusion can have a direct effect on the amount of DUX4 being detected in the assay (FIG. 6).

### Example 7—CK1 Inhibitors Do Not Inhibit Myotube Fusion

Because DUX4 expression increases upon in vitro differentiation of proliferating FSHD myoblasts into multinucleated myotubes (Balog et al., 2015 Epigenetics. 2015; 10(12):1133-42), inhibition of differentiation might lead to a false positive effect on DUX4 repression.

Bromo- and Extra-Terminal domain (BET) inhibitors such as the non-selective inhibitor (+)JQ1 or the BRD4-selective inhibitor RVX-208 can inhibit the expression of DUX4 in immortalised differentiated myotube cultures (see US2015087636A1). It was shown there that when differentiating myotubes were exposed to (+)JQ1 at the start of the differentiation process, i.e. from the moment when the growth medium was changed to the differentiation medium, the expression of myosin heavy chain (MYH2, a differentiation marker) was decreased, suggesting that the inhibitor also impacted the differentiation process. Both (+)JQ1 and RVX-208 have been evaluated in the phenotypic assay described in this application. Agonists of the beta2 adrenoreceptor have also been reported to inhibit DUX4 expression in differentiating myotubes (Campbell et al., 2017) and more recently have been shown to inhibit myotube fusion (Chen et al., 2019, doi.org/10.1186/s13287-019-1160-x; Kim et al., 2019, doi.org/10.1080/19768354.2018.1561516). We evaluated the effect of both BET inhbitors and beta2 adrenoreceptor agonists on the fusion process and compared in to the effect of a CK1 inhibitor.

FIG. 7A shows the experimental setup of Example 2. Compounds are administered either 15 h before fixation, resembling the original screening protocol, or 72 h before fixation (grey arrow). In the latter case, compounds are present during the whole differentiation process. The inventors found that early administration of the BET inhibitor (+)JQ1 (FIG. 7B) and agonists of the beta2 adrenoreceptor (FIGS. 7C,D,E) inhibit the fusion process and the differentiation of myoblasts into myotubes. FIG. 7F shows that no myotube formation can be observed after treatment with a beta2 adrenoreceptor agonist (formoterol). This could lead to a false positive readout when assessing the DUX4 signal. The BET inhibitor RVX-208 did not show any effect on DUX4 expression, irrespective of treatment time (not shown). While the fusion index did not appear to be affected at the 15 h timepoint, also with this treatment time the myotube fusion process was affected by these compounds as determined by RT-PCR showing inhibition of the expression of the late differentiation marker myosin heavy chain (Myh; not shown; primers were from hMYH2 kit described above).

As illustrated in example 5, inhibition of CK1 inhibits DUX4. This effect occurs without inhibiting myotube fusion, neither after 15 h nor after 72 h of compound treatment (FIG. 7G). Table 4 shows half maximal effective concentrations (EC₅₀) values of multiple CK1 inhibitors on DUX4 inhibition in the 72 h compound treatment protocol. Table 4 also shows determined IC₅₀ values in nM for CK1α, CK1δ, CK1ε, and p38a, denoted as CK1 a, d, e, and p38a, respectively.

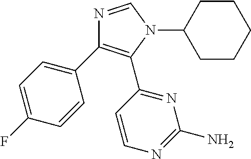 [SMILES: Nc1nccc(-c2c(-c3ccc(F)cc3)ncn2C2CCCCC2)n1]

PF-670462 (EC₅₀ 0.76 μM)
CK1 a: 320; d: 29.1; e: 99.8; p38a: 32.4

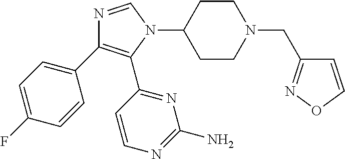 [SMILES: Nc1nccc(-c2c(-c3ccc(F)cc3)ncn2C2CCN(Cc3ccon3)CC2)n1]

PF-5006739 (EC₅₀ 0.62 μM)
CK1 a: 123; d: 19.8; e: 26.8; p38a: 74.3

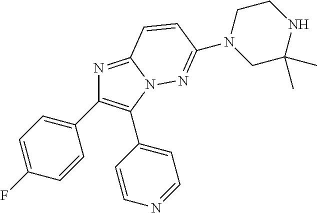 [SMILES: CC1(C)CN(c2ccc3nc(-c4ccc(F)cc4)c(-c4ccncc4)n3n2)CCN1]

1 (EC50 0.04 μM)
CK1 a: 29.5; d: 18.5; e: 12.4; p38a: 13.2

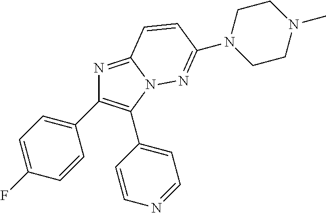 [SMILES: CN1CCN(c2ccc3nc(-c4ccc(F)cc4)c(-c4ccncc4)n3n2)CC1]

2 (EC₅₀ 0.34 μM)
CK1 a: 65; d: 29; p38a: 23

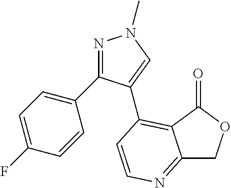 [SMILES: Cn1cc(-c2ccnc3c2C(=O)OC3)c(-c2ccc(F)cc2)n1]

Nr. 4 (EC₅₀ 4.8 μM)
CK1 a: 644; d: 33.1; e: 51.6; p38a: 569

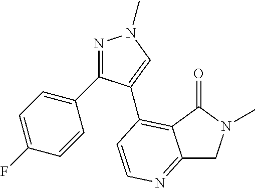 [SMILES: CN1Cc2nccc(-c3cn(C)nc3-c3ccc(F)cc3)c2C1=O]

Nr. 5 (EC₅₀ 6.7 μM)
CK1 a: 592; d: 30.7; e: 83.6; p38a: 1110

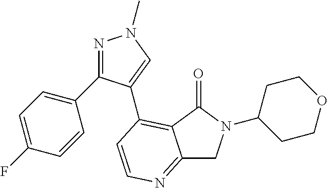 [SMILES: Cn1cc(-c2ccnc3c2C(=O)N(C2CCOCC2)C3)c(-c2ccc(F)cc2)n1]

Nr. 6 (EC₅₀ 8.1 μM)
CK1 a: 561; d: 18; e: 72.4; p38a: 677

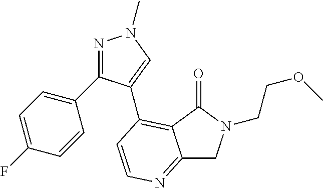 [SMILES: COCCN1Cc2nccc(-c3cn(C)nc3-c3ccc(F)cc3)c2C1=O]

Nr. 7 (EC₅₀ 5.0 μM)
CK1 a: 2590; d: 41.8; e: 92.1; p38a: 712

TABLE 4
Exemplary CK1 inhibitors for use according to the invention, along
with half maximal effective concentrations (EC₅₀). DUX4 EC₅₀
values were obtained in the 72 h treatment protocol

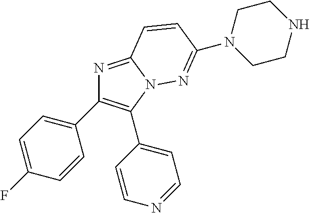 [SMILES: Fc1ccc(-c2nc3ccc(N4CCNCC4)nn3c2-c2ccncc2)cc1]

Nr. 8 (EC₅₀ 0.01-0.06 μM)
CK1 a: 22; d: 16.5; e: 9.41; p38a: 14.8

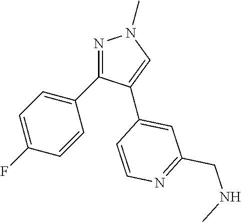 [SMILES: CNCc1cc(-c2cn(C)nc2-c2ccc(F)cc2)ccn1]

Nr. 9 (EC₅₀ 6.4 μM)
CK1 a: 1760; d: 57.7; e: 89; p38a: 3070

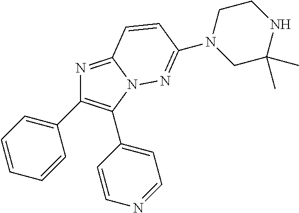 [SMILES: CC1(C)CN(c2ccc3nc(-c4ccccc4)c(-c4ccncc4)n3n2)CCN1]

Nr. 10 (EC₅₀ 0.01 μM)

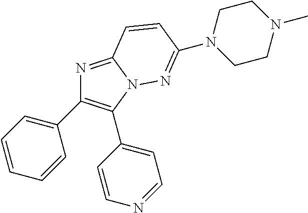 [SMILES: CN1CCN(c2ccc3nc(-c4ccccc4)c(-c4ccncc4)n3n2)CC1]

Nr. 11 (EC₅₀ 0.25 μM)

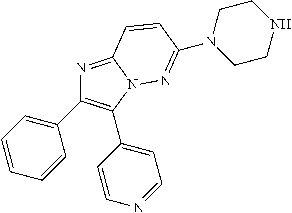 [SMILES: c1ccc(-c2nc3ccc(N4CCNCC4)nn3c2-c2ccncc2)cc1]

Nr. 12 (EC₅₀ 0.05 μM)

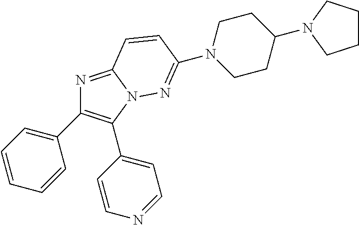 [SMILES: c1ccc(-c2nc3ccc(N4CCC(N5CCCC5)CC4)nn3c2-c2ccncc2)cc1]

Nr. 13 (EC₅₀ 0.31 μM)

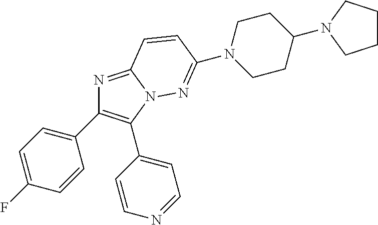 [SMILES: Fc1ccc(-c2nc3ccc(N4CCC(N5CCCC5)CC4)nn3c2-c2ccncc2)cc1]

Nr. 14 (EC₅₀ 0.39 μM)
CK1 a: 270; d: 45; p38a: 42

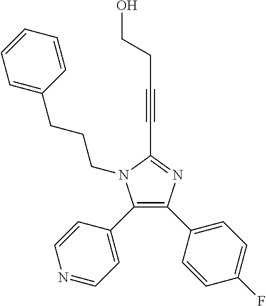 [SMILES: OCCC#Cc1nc(-c2ccc(F)cc2)c(-c2ccncc2)n1CCCc1ccccc1]

RWJ 67657 (EC₅₀: 0.78 μM)
CK1 a: 1830; d: 115; p38a: 15.9

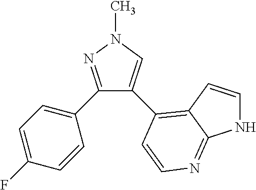 [SMILES: Cn1cc(-c2ccnc3[nH]ccc23)c(-c2ccc(F)cc2)n1]

Nr. 16 (DUX4 EC₅₀: 1.2 μM)
CK1 a: 196; d: 16.9; p38a: 17.9

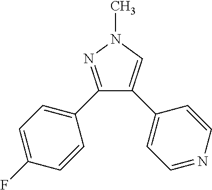 [SMILES: Cn1cc(-c2ccncc2)c(-c2ccc(F)cc2)n1]

Nr. 17 (EC₅₀: 2.9 μM)
CK1 a: 873; d: 36.1; p38a: 63.2

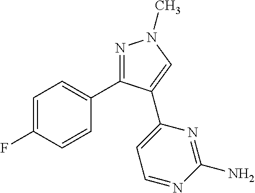 [SMILES: Cn1cc(-c2ccnc(N)n2)c(-c2ccc(F)cc2)n1]

Nr. 18 (EC₅₀: 8.2 μM)
CK1 a: 636; d: 46.5; p38a: 312

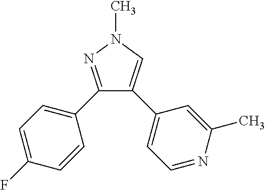 [SMILES: Cc1cc(-c2cn(C)nc2-c2ccc(F)cc2)ccn1]

Nr. 19 (EC₅₀: 4.9 μM)
CK1 a: 4020; d: 80.7; p38a: 449

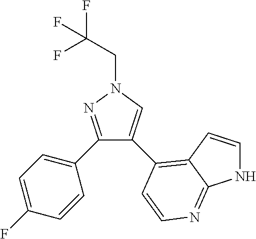 [SMILES: Fc1ccc(-c2nn(CC(F)(F)F)cc2-c2ccnc3[nH]ccc23)cc1]

Nr. 20 (EC₅₀: 2.2 μM)
CK1 a: 186; d: 55.5; p38a: 50.3

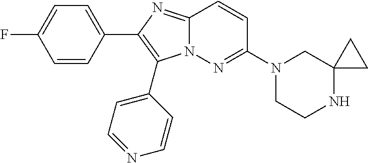 [SMILES: Fc1ccc(-c2nc3ccc(N4CCNC5(CC5)C4)nn3c2-c2ccncc2)cc1]

Nr. 21 (EC₅₀: 0.1 μM)
CK1 a: 61.4; d: 58.5; p38a: 28.9

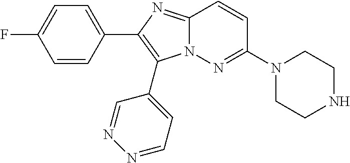 [SMILES: Fc1ccc(-c2nc3ccc(N4CCNCC4)nn3c2-c2ccnnc2)cc1]

Nr. 22 (EC₅₀: 1.1 μM)
CK1 a: 194; d: 57.7; p38a: 411

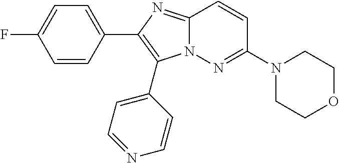 [SMILES: Fc1ccc(-c2nc3ccc(N4CCOCC4)nn3c2-c2ccncc2)cc1]

Nr. 23 (EC₅₀: 2.4 μM)
CK1 a: >10000; d: 2090; p38a: 1250

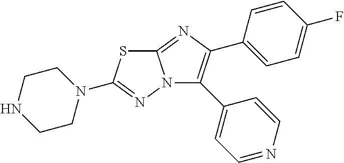 [SMILES: Fc1ccc(-c2nc3sc(N4CCNCC4)nn3c2-c2ccncc2)cc1]

Nr. 24 (EC₅₀: 0.48 μM)
CK1 a: 331; d: 55.7; p38a: 21.7

### Example 8—CK1 Inhibitors Inhibition Profile

Compounds PF-670462, PF-5006739, Compound E, Compound F, Compound D, Compound H, Compound A, and SR3029 were assayed for their inhibition of CK1α, CK1δ, CK1ε, and of p38, and their concurrent repression of DUX4. Table 5 and 6 show inhibitory results.

TABLE 5
inhibition of CK1 and p38 by CK1 inhibitors, in nM. DUX4 EC₅₀ values
were obtained in the 15 h treatment protocol

| IC₅₀ | PF- | PF- | | | | | | SR- |
|---|---|---|---|---|---|---|---|---|
| ECso | 670462 | 5006739 | 5 | 6 | 4 | 8 | 1 | 3029 |
| CK1 α | 320 | 123 | 592 | 561 | 644 | 33 | 30 | >10k |
| CK1 δ | 29 | 20 | 31 | 18 | 33.1 | 22 | 19 | 346 |
| CK1 ε | 100 | 27 | 84 | 72 | 51.6 | 16 | 12 | 381 |
| p38 | 32 | 74 | 1110 | 677 | 569 | 25 | 13 | >10k |
| DUX4 | 470 | 820 | 1890 | 2590 | 1410 | 10 | 50 | 50 |
| | (n = 4) | (n = 12) | (n = 4) | (n = 2) | (n = 2) | (n = 2) | (n = 2) | |

TABLE 6
inhibition of CK1 and p38 by CK1 inhibitors, in nM.
DUX4 EC50 values were obtained in the 72 h treatment protocol

| IC₅₀ | | | | | |
|---|---|---|---|---|---|
| EC₅₀ | CK1α | CK1δ | CK1ε | p38α | DUX4 |
| PF-670462 | 320 | 29 | 100 | 32 | 760 |
| PF-5006739 | 123 | 20 | 27 | 74 | 620 |
| 1 | 29 | 18 | 12 | 13 | 40 |
| 2 | 65 | 29 | | 23 | 340 |
| 4 | 644 | 33 | 52 | 569 | 4800 |
| 5 | 592 | 31 | 84 | 1110 | 6700 |
| 6 | 561 | 18 | 72 | 677 | 8100 |
| 7 | 2590 | 42 | 92 | 712 | 5000 |
| 8 | 22 | 16 | 9 | 15 | 10 |
| 9 | 1760 | 58 | 89 | 3070 | 6400 |
| 10 | Na | Na | Na | Na | 10 |
| 11 | Na | Na | Na | Na | 250 |
| 12 | Na | Na | Na | Na | 50 |
| 13 | Na | Na | Na | Na | 310 |
| 14 | 270 | 45 | Na | 42 | 390 |
| 15 | 1830 | 115 | Na | 16 | 780 |
| 16 | 196 | 17 | Na | 18 | 1200 |
| 17 | 873 | 36 | Na | 63 | 2900 |
| 18 | 636 | 46 | Na | 312 | 8200 |
| 19 | 4020 | 81 | Na | 449 | 4900 |
| 20 | 186 | 55 | Na | 50 | 2200 |
| 21 | 61 | 58 | Na | 29 | 100 |
| 22 | 194 | 58 | Na | 411 | 1100 |
| 23 | >10000 | 2090 | Na | 1250 | 2400 |
| 24 | 331 | 58 | Na | 22 | 480 |

### Example 9—p38 Inhibitors Inhibit Fusion of Primary Myoblasts From FSHD Donors

Because DUX4 expression increases upon in vitro differentiation of proliferating FSHD myoblasts into multinucleated myotubes (Balog et al., 2015 Epigenetics. 2015; 10(12):1133-42), inhibition of differentiation might lead to a false positive effect on DUX4 repression. Recently, p38 have been described to inhibit DUX4 mRNA expression without affecting the myogenic differentiation markers MYOG and MYH2 (WO2019/071144 and WO2019/071147). Since the expression of these markers does not necessarily correlate with fusion, we analysed a series of p38 inhibitors in the high content assay and quantified their effects on DUX4 expression and myotube fusion. Since losmapimod did not show any effect on DUX4, nor the fusion index, when it was added during the last 15 h of differentiation prior to fixation (not shown), all experiments were performed by administering the compounds when the growth medium was replaced with differentiation medium, 72 h before cells were fixed. As illustrated in FIG. 8, all tested p38 inhibitors (losmapimod, BMS-582949, pexmetinib or ARRY-614, BIRB796, SCIO469, PH797804, pamapimod, LY2228820, R1487, SB-681323, VX-745, acumapimod, VX702) inhibited the fusion index, largely obscuring effects on DUX4, if any. Using losmapimod, this inhibitory effect on the fusion index was confirmed in a primary FSHD muscle cell from a different donor (not shown).

TABLE 7
IC50 values of different p38 compounds on p38a, CK1a and CK1d

| Compound | p38 (nM) | CK1a/CK1d (nM) |
|---|---|---|
| Acumapimod | 22 | >10,000/>10,0000 |
| AMG548 | 7 | >10,000/452 |
| BIRB795 | 99 | >10,000/>10,0000 |
| BMS-582949 | 65 | >10,000/>10,0000 |
| Losmapimod | 26 | >10,000/>10,0000 |
| LY2228820 | 14 | >10,000/2780 |
| Pamapimod | 16 | >10,000/>10,0000 |
| pexmetinib | 17 | >10,000/>10,0000 |
| PH797804 | 7 | >10,000/>10,0000 |
| R1487 | 13 | >10,000/9430 |
| SB-681323 | 11 | >10,000/>10,0000 |
| SCIO469 | 16 | >10,000/>10,0000 |
| VX702 | 25 | >10,000/>10,0000 |
| VX745 | 29 | >10,000/>10,0000 |

### Example 10—P38 Inhibitors Inhibit Fusion of Primary Myoblasts From Healthy Donors

As illustrated in FIG. 9, the inhibitory effect of p38 inhibitors was not restricted to FSHD cell lines. A primary muscle cell from a healthy donor was treated as described in example 2, with the exception that it was allowed to differentiate for five days rather than 3 days to account for the slower differentiation time (time to reach maximal fusion index). Losmapimod, was added when the growth medium was changed to differentiation medium. Under these conditions, losmapimod clearly inhibited the fusion index, reflecting inhibited formation of multinucleated myotubes.

### Example 11. CK1 Inhibitors Protect Against Fusion Inhibition Caused By p38 Inhibitors in Primary Myotubes

As shown in example 9 and 10, and FIG. 10 A, treating differentiating primary myotubes with a p38 inhibitor (72 h protocol) prevents them to form multinucleated fused myotubes. The inventors have found that when the cells are treated with a p38 inhibitor in the presence of a CK1 inhibitor, which in its own does not inhibit myotube fusion (FIG. 10 A, B, D, F), the deleterious effect on fusion can, at least partially, be prevented. FIG. 10A shows a comparison of the effect of losmapimod and different CK1 inhibitors on fusion at different concentrations. When myotubes are differentiated in the presence of increasing concentrations of losmapimod, either alone or in the presence of either compound Nr.4, Nr.5 or Nr.8, it is clear from the microscopic images that in the presence of compounds Nr.4, Nr.5 or Nr.8, myotube formation is still intact, also at the higher losmapimod concentrations (FIG. 10 C, F, I). Similarly, FIG. 10 (D, G, J) shows that losmapimod has a concentration-dependent inhibition of myotube fusion. However, in the presence of a CK1 inhibitor, the inhibition of myotube fusion by losmapimod is, at least partially, prevented. This is also obvious from an experiment where a single combination of the p38 inhibitor losmapimod is combined with increasing concentrations of a CK1 inhibitor (FIG. 10 K, L, M). The fusion inhibition by losmapimod is concentration-dependently inhibited by the CK1 inhibitors.

### Example 12. The inhibitory Potential on DUX4 By a CK1 Inhibitor is Retained in the Presence of a p38 Inhibitor

When a CK1 inhibitor is combined with increasing concentrations of losmapimod, it not only protects against inhibition of myotube fusion, but also retains its capacity to inhibit DUX4 (FIG. 11A, B, C). Even more, the treatment of primary FSHD myotubes with a combination of a CK1 inhibitor and a p38 inhibitor induces a stronger reduction of DUX4 than a CK1 inhibitor alone (FIG. 11A, B). This is less obvious for compound Nr.8 because it already induces near maximal DUX4 inhibition in the absence of the p38 inhibitor (FIG. 110).

### Example 13. Dual CK1/p38 Inhibitors Inhibit DUX4 Expression Without Affecting Myotube Formation

The protective effect of CK1 inhibition on myotube fusion also clearly shows from the profiles of CK1 inhibitors that also inhibit p38 (Table 6). As illustrated for PF-670462 in FIG. 7G2, these dual inhibitors repress DUX4 without inhibiting the fusion index, illustrating that they do not affect myotube formation.

### REFERENCES

Balog et al., 2015 Epigenetics. 2015; 10(12):1133-42; Bergerat et al., 2017, DOI: 10.1016/j.prp.2016.11.015; Van den Boogaard et al., 2016, DOI: 10.1016/j.ajhg.2016.03.013; Brockschmidt et al., 2008, DOI: 10.1136/gut.2007.123695; Campbell et al., 2017, DOI: 10.1186/s13395-017-0134-x; Chebib and Jo, 2016, DOI: 10.1002/cncy.21685; Eide E J, Virshup DM, 2001, DOI:10.1081/CBI-100103963; Etchegaray J P et al., 2009, DOI:10.1128/MCB.00338-09; Geng et al., 2012, DOI: 10.1016/j.devce1.2011.11.013; Kowaljow et al., 2007, DOI: 10.1016/j.nmd.2007.04.002; Lang et al., 2014, DOI: 10.14205/2310-8703.2014.02.01.1; Lemmers et al., 2010, DOI: 10.1126/science.1189044; Lilljebjörn & Fioretos, 2017, DOI: 10.1182/blood-2017-05-742643; Oyama et al., 2017 DOI: 10.1038/s41598-017-04967-0; Paz et al., 2003, DOI: 10.1093/hmg/ddg226; Rickard et al., 2015, DOI: 10.1093/hmg/ddv315; Rudnicki et al., 1993; cell 75(7):1351-9; Sharma et al., 2016, DOI:10.4172/2157-7412.1000303; Snider et al., 2010, DOI: 10.1371/journal.pgen.1001181; Stadler et al., 2013, DOI: 10.1038/nsmb.2571; Tawil et al., 2014, DOI: 10.1186/2044-5040-4-12; Vanderplanck et al., 2011, doi: 10.1371/journal.pone.0026820; Wallace et al., 2011, DOI: 10.1002/ana.22275; Yao et al., 2014, DOI: 10.1093/hmg/ddu251; Yasuda et al., 2016, doi: 10.1038/ng.3535; Young et al., 2013, doi:10.1371/journal.pgen.1003947; Zhang et al., 1999, doi:10.1177/108705719900400206; Zhang et al., 2017, DOI:10.1038/ng.3691 WO2011051858/WO2012085721/WO2015119579/EP2949651/WO2009016286/US2005/0131012/WO2015195880/WO2014081923/US20140221313/US2015087636A1

1-15. (canceled)

16. A method of treating a disease or condition associated with DUX4 expression in a subject in need thereof, the method comprising the step of administering a casein kinase 1 inhibitor to the subject, wherein the casein kinase 1 inhibitor is for promoting myogenic fusion and/or differentiation.

17. The method of claim 16, wherein the subject suffers from muscle inflammation.

18. The method of claim 16, wherein the disease or condition associated with DUX4 expression is a muscular dystrophy or cancer.

19. The method of claim 18, wherein the disease or condition associated with DUX4 expression is facioscapulohumeral muscular dystrophy (FSHD).

20. The method of claim 16, wherein the casein kinase inhibitor inhibits at least casein kinase 1δ.

21. The method of claim 16, wherein the treatment reduces DUX4 expression in the subject by at least 20%, 40%, 60%, 80%, or more.

22. A method of treating a disease or condition associated with DUX4 expression in a subject in need thereof, the method comprising the step of administering a combination of a casein kinase 1 inhibitor and a p38 inhibitor to the subject.

23. The method of claim 22, wherein the subject suffers from muscle inflammation.

24. The method of claim 22, wherein the casein kinase 1 inhibitor is for promoting myogenic fusion and/or differentiation.

25. The method of claim 24, wherein the subject suffers from muscle inflammation.

26. The method of claim 24, wherein the casein kinase 1 inhibitor and the p38 inhibitor are two distinct substances.

27. The method of claim 24, wherein the casein kinase 1 inhibitor and the p38 inhibitor are the same substance.

28. The method of claim 24, wherein the disease or condition associated with DUX4 expression is a muscular dystrophy or cancer.

29. The method of claim 28, wherein the disease or condition associated with DUX4 expression is a muscular dystrophy.

30. The method of claim 29, wherein said disease or condition associated with DUX4 expression is facioscapulohumeral muscular dystrophy (FSHD)

31. The method of claim 22, wherein:
a) the p38 inhibitor inhibits at least p38α, and/or
b) the casein kinase inhibitor inhibits at least casein kinase 1δ.

32. The method of claim 22, wherein the treatment reduces DUX4 expression in the subject by at least 20%, 40%, 60%, 80%, or more.

33. An in vivo, in vitro, or ex vivo method for promoting myogenic fusion and/or differentiation, the method comprising the step of contacting a cell with a casein kinase 1 inhibitor, or with a combination of a casein kinase 1 inhibitor and a p38 inhibitor.

34. The method of claim 33, wherein the casein kinase 1 inhibitor inhibits at least casein kinase 1δ.

35. The method of claim 33, wherein p38 inhibitor inhibits at least p38α.


SEQUENCE LISTING (15 sequences)

SEQ ID NO: 1 - DNA, 16 bp, Artificial Sequence
  hDUX4 forward Primer for qPCR
cccggctgacgtgcaa

SEQ ID NO: 2 - DNA, 22 bp, Artificial Sequence
  hDUX4 reverse Primer for qPCR
agccagaatttcacggaagaac

SEQ ID NO: 3 - DNA, 22 bp, Artificial Sequence
  hDUX4 probe Primer for qPCR
agctcgctggcctctctgtgcc

SEQ ID NO: 4 - DNA, 21 bp, Artificial Sequence
  hGUSB forward Primer for qPCR
ttccctccagcttcaatgaca

SEQ ID NO: 5 - DNA, 17 bp, Artificial Sequence
  hGUSB reverse Primer for qPCR
ccacacccagccgacaa

SEQ ID NO: 6 - DNA, 19 bp, Artificial Sequence
  hGUSB probe Primer for qPCR
aggactggcgtctgcggca

SEQ ID NO: 7 - DNA, 20 bp, Artificial Sequence
  hRPL27 forward Primer for qPCR
tgtcctggctggacgctact

SEQ ID NO: 8 - DNA, 23 bp, Artificial Sequence
  hRPL27 reverse Primer for qPCR
gaggtgccatcatcaatgttctt

SEQ ID NO: 9 - DNA, 21 bp, Artificial Sequence
  hRPL27 probe Primer for qPCR
cggacgcaaagctgtcatcgt

SEQ ID NO: 10 - DNA, 22 bp, Artificial Sequence
  hZSCAN4 forward Primer for qPCR
aggcaggaattgcaaagacttt

SEQ ID NO: 11 - DNA, 23 bp, Artificial Sequence
  hZSCAN4 reverse Primer for qPCR
aatttcatccttgctgtgctttt

SEQ ID NO: 12 - DNA, 28 bp, Artificial Sequence
  hZSCAN4 probe Primer for qPCR
taggatctttcactcatggctgcaacca

SEQ ID NO: 13 - DNA, 19 bp, Artificial Sequence
  hMYOG forward Primer for qPCR
gctcacggctgaccctaca

SEQ ID NO: 14 - DNA, 21 bp, Artificial Sequence
  hMYOG reverse Primer for qPCR
cactgtgatgctgtccacgat

SEQ ID NO: 15 - DNA, 24 bp, Artificial Sequence
  hMYOG probe Primer for qPCR
cccacaacctgcactccctcacct